(19) United States
(12) Patent Application Publication
(10) Pub. No.: US 2013/0096141 A1
(43) Pub. Date: Apr. 18, 2013

(54) BICYCLIC HETEROCYCLE DERIVATIVES AND METHODS OF USE THEREOF

(71) Applicants: Bernard R. Neustadt, West Orange, NJ (US); Hong Liu, Hillsborough, NJ (US)

(72) Inventors: Bernard R. Neustadt, West Orange, NJ (US); Hong Liu, Hillsborough, NJ (US)

(21) Appl. No.: 13/805,054

(22) PCT Filed: Jun. 14, 2011
(86) PCT No.: PCT/US11/40276

§ 371 Date: Dec. 18, 2012

Related U.S. Application Data
(60) Provisional application No. 61/356,262, filed on Jun. 18, 2010.

(51) Int. Cl. C07D 519/00

(52) U.S. Cl. CPC C07D 519/00
USPC 514/260.1; 544/278; 544/280; 514/265.1

(57) ABSTRACT

Compounds of structural formula I: are GPR119 agonists and are useful for the treatment, control or prevention of disorders responsive to agonism of GPR119, such as metabolic-related disorders such as type I diabetes, type II diabetes, inadequate glucose tolerance, insulin resistance, hyperglycemia, hyperlipidemia, hypertriglyceridemia, hypercholesterolemia, dyslipidemia or syndrome X. The compounds are also reported as being useful for controlling weight gain, controlling food intake, and inducing satiety in mammals.

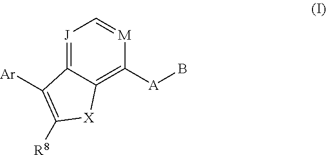

### FIELD OF THE INVENTION

The present invention relates to bicyclic heterocycle derivatives, compositions comprising a bicyclic heterocycle derivative, and methods of using the bicyclic heterocycle derivatives for treating or preventing obesity, diabetes, a diabetic complication, a metabolic disorder, a cardiovascular disease or a disorder related to the activity of a G protein-coupled receptor (GPCR) in a patient.

### BACKGROUND

Although a number of receptor classes exist in humans, by far the most abundant and therapeutically relevant is represented by the G protein-coupled receptor class. It is estimated that there are some 100,000 genes within the human genome, and of these, approximately 2% or 2,000 genes, are estimated to code for GPCRs. Receptors, including GPCRs, for which the endogenous ligand has been identified are referred to as “known” receptors, while receptors for which the endogenous ligand has not been identified are referred to as “orphan” receptors. GPCRs represent an important area for the development of pharmaceutical products, as evidenced by the fact that pharmaceutical products have been developed from approximately 20 of the 100 known GPCRs. This distinction is not merely semantic, particularly in the case of GPCRs. Thus, the orphan GPCRs are to the pharmaceutical industry what gold was to California in the late 19th century—an opportunity to drive growth, expansion, enhancement and development.

GPCRs share a common structural motif. All these receptors have seven sequences of between 22 to 24 hydrophobic amino acids that form seven alpha helices, each of which spans the membrane (each span is identified by number, i.e., transmembrane-1 (TM-1), transmembrane-2 (TM-2), etc.). The transmembrane helices are joined by strands of amino acids between transmembrane-2 and transmembrane-3, transmembrane-4 and transmembrane-5, and transmembrane-6 and transmembrane-7 on the exterior, or “extracellular” side, of the cell membrane (these are referred to as “extracellular” regions 1, 2 and 3 (EC-1, EC-2 and EC-3), respectively). The transmembrane helices are also joined by strands of amino acids between transmembrane-1 and transmembrane-2, transmembrane-3 and transmembrane-4, and transmembrane-5 and transmembrane-6 on the interior, or “intracellular” side, of the cell membrane (these are referred to as “intracellular” regions 1, 2 and 3 (IC-1, IC-2 and IC-3), respectively). The “carboxy” (“C”) terminus of the receptor lies in the intracellular space within the cell, and the “amino” (“N”) terminus of the receptor lies in the extracellular space outside of the cell.

Generally, when an endogenous ligand binds with the receptor (often referred to as “activation” of the receptor), there is a change in the conformation of the intracellular region that allows for coupling between the intracellular region and an intracellular “G-protein.” It has been reported that GPCRs are “promiscuous” with respect to G proteins, i.e., that a GPCR can interact with more than one G protein. See, Kenakin, T., Life Sciences 43, 1095 (1988). Although other G proteins exist, currently, Gq, Gs, Gi, and Go are G proteins that have been identified. Endogenous ligand-activated GPCR coupling with the G-protein begins a signaling cascade process (referred to as “signal transduction”). Under normal conditions, signal transduction ultimately results in cellular activation or cellular inhibition. It is thought that the IC-3 loop as well as the carboxy terminus of the receptor interact with the G protein.

Under physiological conditions, GPCRs exist in the cell membrane in equilibrium between two different conformations: an “inactive” state and an “active” state. A receptor in an inactive state is unable to link to the intracellular signaling transduction pathway to produce a biological response. Changing the receptor conformation to the active state allows linkage to the transduction pathway (via the G-protein) and produces a biological response. A receptor can be stabilized in an active state by an endogenous ligand or a compound such as a drug.

Modulation of G-protein coupled receptors has been well-studied for controlling various metabolic disorders. Small molecule modulators of the receptor GPR119, a O-protein coupled-receptor described in, for example, GenBank (see, e.g., accession numbers XM.sub.-066873 and AY288416), have been shown to be useful for treating or preventing certain metabolic disorders. GPR119 is a G protein-coupled receptor that is selectively expressed on pancreatic beta cells. GPR119 activation leads to elevation of a level of intracellular cAMP, consistent with GPR119 being coupled to Gs. Agonists to GPR119 stimulate glucose-dependent insulin secretion in vitro and lower an elevated blood glucose level in vivo. See, e.g., International Publication Nos. WO 04/065380, WO 04/076413, and EP 1338651, the disclosure of each of which is herein incorporated by reference in its entirety.

U.S. Ser. No. 10/890,549 discloses pyrazolo[3,4-d]pyrimidine ethers and related compounds as modulators of the GPR119 receptor that are useful for the treatment of various metabolic-related disorders such as type I diabetes, type II diabetes, inadequate glucose tolerance, insulin resistance, hyperglycemia, hyperlipidemia, hypertriglyceridemia, hypercholesterolemia, dyslipidemia or syndrome X. The compounds are also reported as being useful for controlling weight gain, controlling food intake, and inducing satiety in mammals. The promising nature of these GPCR modulators indicates a need in the art for additional small molecule GPCR modulators with improved efficacy and safety profiles. This invention addresses that need.

### SUMMARY

The present invention is directed to compounds of structural formula I:

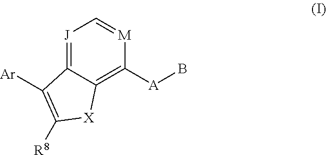

or a pharmaceutically acceptable salt thereof, wherein:

A is —O—, —S—, —NH—, or —N(C₁-C₆alkyl)-;

B is:

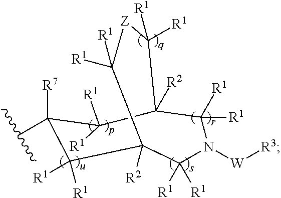

J is —C(R¹¹)— or —N—;

M is —C(R¹¹)— or —N—;

W is a bond, C₁-C₆alkyl, —C(O)—, —C(O)—O—, —S(O)₂—, —S(O)₂—N(R¹⁰)—, C(S)O or —C(O)—N(R¹⁰)—;

X is —O—, —S—, —NH, —N(C₁-C₆alkyl), —N(cycloalkyl), —N(hydroxyalkyl) or —N(hydroxyaryl);

Z is a bond, —C(O)—, —C═NOR¹², —C═C(R¹⁴)₂, —C(R¹)₂—, —O—, —N(R¹⁰)— or —S(O)ₙ—;

each occurrence of R¹ is independently hydrogen, C₁-C₆alkyl, cycloalkyl, halogen, haloalkyl or —OR⁷, wherein OR⁷ is not adjacent to —N—W—R³;

each occurrence of R² is independently hydrogen or C₁-C₆alkyl;

R³ is C₁-C₆alkyl, C₂-C₆alkenyl, C₂-C₆alkynyl, haloalkyl, —(C₁-C₆alkyl)ₜ-cycloalkyl, —(C₁-C₆alkyl)ₜ-heterocycloalkyl, —(C₁-C₆alkyl)ₜ-aryl or —(C₁-C₆alkyl)ₜ-heteroatyl, wherein the cycloalkyl, heterocycloalkyl, aryl or heteroaryl group can be unsubstituted or substituted with one or more substituents each independently selected from R⁹;

each occurrence of R⁴ is independently hydrogen or C₁-C₆alkyl;

each occurrence of R⁷ is independently hydrogen or C₁-C₆alkyl;

Ar is aryl, heteroaryl, heterocycloalkyl or cycloalkyl, any of which can be unsubstituted or substituted with one or more substituents each independently selected from R⁹;

R⁸ is hydrogen, halogen, C₁-C₆alkyl or cycloalkyl;

R⁹ represents C₁-C₆alkyl, C₂-C₆alkenyl, C₂-C₆alkynyl, halogen, haloalkyl, —CN, —NO₂, —O—(C₁-C₆alkyl)ₜ-R¹³, —S—(C₁-C₆alkyl)ₜ-R¹³, —N(R¹³)—(C₁-C₆alkyl)ₜ-R¹³, —(C₁-C₆alkyDrR¹³, —C(O)—(C₁-C₆alkyl)ₜ-R¹³, —C(O)O—(C₁-C₆alkyl)ₜ-R¹³, —N(R⁷)C(O)—(C₁-C₆alkyl)ₜ-R¹³, —C(O)N(R⁷)—(C₁-C₆alkyl)ₜ-R¹³, —OC(O)—(C₁-C₆alkyl)ₜ-R¹³, —N(R⁷)C(O)NR⁷)—(C₁-C₆alkyl)ₜ-R¹³, —N(R⁷)C(O)O—(C₁-C₆alkyl)ₜ-R¹³, —S(O)—(C₁-C₆alkyl)ₜ-R¹³ or —S(O)₂(C₁-C₆alkyl)ₜ-R¹³;

R¹⁰ is hydrogen, C₁-C₆alkyl, aryl, or —C(O)OR⁴;

each occurrence of R¹¹ is independently hydrogen, C₁-C₆alkyl, aryl, cycloalkyl, heterocycloalkyl, heteroaryl, —N(R⁷)₂ or halogen;

each occurrence of R¹² is independently hydrogen, C₁-C₆alkyl or aryl;

each occurrence of R¹³ is independently hydrogen, hydroxyl, haloalkyl, aryl, cycloalkyl, —COOC₁-C₆alkyl, —OC₁-C₆alkyl or heteroaryl;

each occurrence of R¹⁴ is independently hydrogen, C₁-C₆alkyl or aryl, or both R¹⁴ groups, and the carbon atom to which they are attached, combine to form a cycloalkyl or heterocycloalkyl group;

each occurrence of n, p, q, r, s, t and u is independently 0, 1 or 2.

### DETAILED DESCRIPTION

### Compounds

The present invention is directed to compounds of formula I:

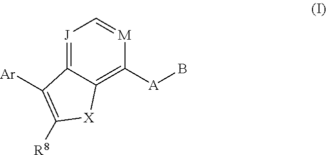

or a pharmaceutically acceptable salt thereof, wherein: A is —O—, —S—, —NH—, or —N(C₁-C₆alkyl)-; B is:

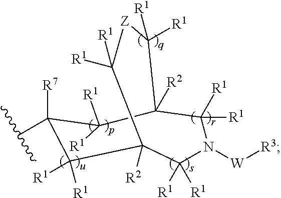

J is —C(R¹¹)— or —N—; M is —C(R¹¹)— or —N—; W is a bond, C₁-C₆alkyl, —C(O)—, —C(O)—O—, —S(O)₂—, —S(O)₂—N(R¹⁰)—, C(S)O or —C(O)—N(R¹⁰)—; X is —O—, —S—, —NH, —N(C₁-C₆alkyl), —N(cycloalkyl), —N(hydroxyalkyl) or —N(hydroxyaryl); Z is a bond, —C(O)—, —C═NOR¹², —C═C(R¹⁴)₂, —C(R¹)₂—, —O—, —N(R¹⁰)— or —S(O)ₙ—; each occurrence of R¹ is independently hydrogen, C₁-C₆alkyl, cycloalkyl, halogen, haloalkyl or —OR⁷, wherein OR⁷ is not adjacent to —N—W—R³; each occurrence of R² is independently hydrogen or C₁-C₆alkyl; R³ is C₁-C₆alkyl, C₂-C₆alkenyl, C₂-C₆alkynyl, haloalkyl, —(C₁-C₆alkyl)ₜ-cycloalkyl, —(C₁-C₆alkyl)ₜ-heterocycloalkyl, —(C₁-C₆alkyl)ₜ-aryl or —(C₁-C₆alkyl)ₜ-heteroaryl, wherein the cycloalkyl, heterocycloalkyl, aryl or heteroaryl group can be unsubstituted or substituted with one or more substituents each independently selected from R⁹; each occurrence of R⁴ is independently hydrogen or C₁-C₆alkyl; each occurrence of R⁷ is independently hydrogen or C₁-C₆alkyl; Ar is aryl, heteroaryl, heterocycloalkyl or cycloalkyl, any of which can be unsubstituted or substituted with one or more substituents each independently selected from R⁹; R⁸ is hydrogen, halogen, C₁-C₆alkyl or cycloalkyl; R⁹ represents C₁-C₆alkyl, C₂-C₆alkenyl, C₂-C₆alkynyl, halogen, haloalkyl, —CN, —NO₂, —O—(C₁-C₆alkyl)ₜ-R¹³, —S—(C₁-C₆alkyl)ₜ-R¹³, —N(R¹³)—(C₁-C₆alkyl)ₜ-R¹³, —(C₁-C₆alkyl)ₜ-R¹³, —C(O)—(C₁-C₆alkyl)ₜ-R¹³, —C(O)O—(C₁-C₆alkyl)ₜ-R¹³, —N(R⁷)C(O)—(C₁-C₆alkyl)ₜ-R¹³, —C(O)N(R⁷)—(C₁-C₆alkyl)ₜ-R¹³, —OC(O)—(C₁-C₆alkyl)ₜ-R¹³, —N(R⁷)C(O)N(R⁷)—(C₁-C₆alkyeₜ-R¹³, —N(R⁷)C(O)O—(C₁-C₆alkyl)ₜ-R¹³, —S(O)—(C₁-C₆alkyl)ₜ-R¹³ or —S(O)₂(C₁-C₆alkyl)ₜ-R¹³; R¹⁰ is hydrogen, C₁-C₆alkyl, aryl, or —C(O)OR⁴; each occurrence of R¹¹ is independently hydrogen, C₁-C₆alkyl, aryl, cycloalkyl, heterocycloalkyl, heteroaryl, —N(R⁷)₂ or halogen; each occurrence of R¹² is independently hydrogen, C₁-C₆alkyl or aryl; each occurrence of R¹³ is independently hydrogen, hydroxyl, haloalkyl, aryl, cycloalkyl, —COOC₁-C₆alkyl, —OC₁-C₆alkyl or heteroaryl; each occurrence of R¹⁴ is independently hydrogen, C₁-C₆alkyl or aryl, or both R¹⁴ groups, and the carbon atom to which they are attached, combine to form a cycloalkyl or heterocycloalkyl group; each occurrence of n, p, q, r, s, t and u is independently 0, 1 or 2,

In certain embodiments of the compounds described herein A is —O—. In other embodiments of the compounds described herein, A is —S—. In still other embodiments of the compounds described herein, A is —NH—. In yet other embodiments of the compounds described herein A is —N(C₁-C₆alkyl)-.

In some embodiments of the compounds described herein, B is:

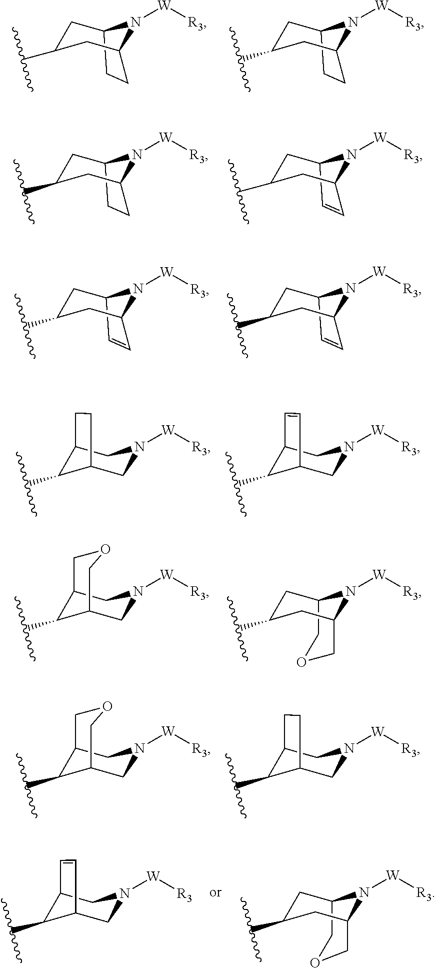

In still another embodiment, B is:

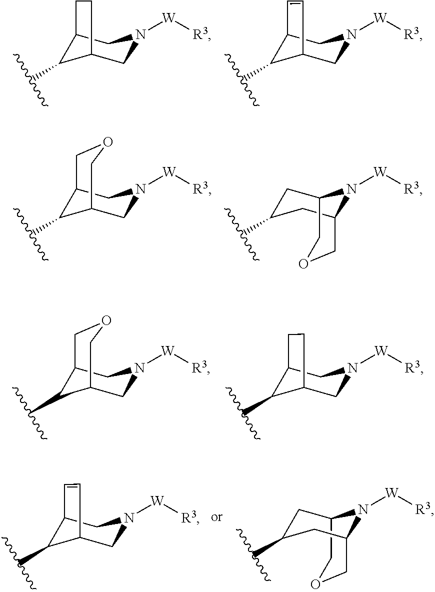

In yet another embodiment B is:

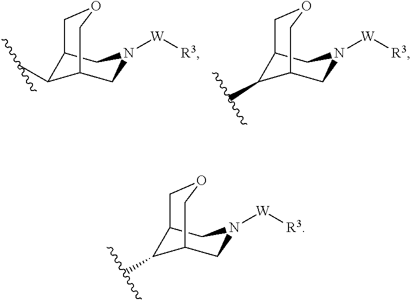

For example in certain embodiments of the compounds described herein, B is:

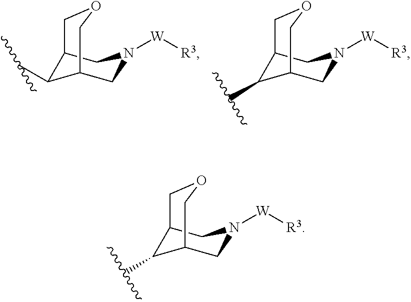

wherein W is as defined above, R³ is C₁-C₆alkyl, haloalkyl, cycloalkyl or heteroaryl, wherein the cycloalkyl and heteroaryl are substituted with one or more substituents selected from R⁹.

As other example of the embodiments of the compounds described herein, B is:

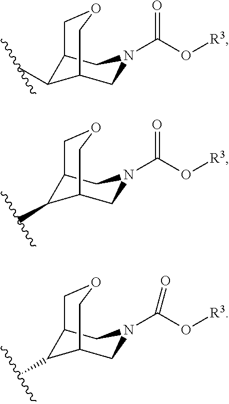

wherein R³ is as defined above.

In yet another example of the embodiments of the compounds described herein B is:

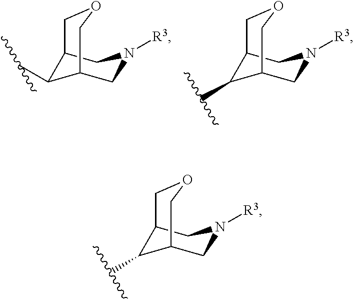

wherein R³ is as defined above.

In regard to the compounds described herein, J and M are independently —N— or —C(R¹¹)—. In one embodiment, J is —C(R¹¹)—. In another embodiment, J is —N—. In another embodiment, J is —CH— or —N—. In still another embodiment, J is —CH—. In one embodiment, M is —C(R¹¹)—. In another embodiment, M is —N—. In another embodiment, M is —CH— or —N—. In still another embodiment, M is —CH—. In yet another embodiment of the compounds described herein, J and M are each —N—.

In regard to the compounds described herein W is a bond, C₁-C₆alkyl, —C(O)—, —C(O)—O—, —S(O)₂—, —S(O)₂—N(R¹⁰)—, C(S)O or —C(O)N(R¹⁰)—. In one embodiment, W is —C(O)O—, —S(O)₂—, —C(O)— or C₁-C₆alkyl. In another embodiment, W is —C(O)O— or —S(O)₂—. In still another embodiment, W is —C(O)O— or —C(O)—. In another embodiment, W is a bond. In still another embodiment, W is C₁-C₆alkyl. In another embodiment, W is —C(O)—. In yet another embodiment, W is —S(O)₂—. In another embodiment, W is —CH₂—. In another embodiment, W is —C(O)O—. In yet another embodiment, W is —S(O)₂N(R¹⁰)—. In a further embodiment, W is —C(O)N(R¹⁰)—.

In regard to the compounds described herein, X is —O—, —S—, —NH, —N(C₁-C₆alkyl), —N(cycloalkyl), —N(hydroxyalkyl) or —N(hydroxyaryl). In one embodiment of the compounds described herein, X is —O—. In another embodiment, X is —S—. In yet another embodiment, X is —NH. In still another embodiment, X is —N(C₁-C₆alkyl). In yet another embodiment, X is —N(cycloalkyl). Examples of cycloalkyl include, but are not limited to, cyclopropyl, cylcobutyl, cyclopentyl and cylcohexyl. In still another embodiment, X is —N(hydroxyaryl). Examples of hydroxyaryl include hydroxyphenyl. In still another embodiment, X is —N(hydroxyalkyl). Examples of hydroxyalkyl include, but are not limited to, 2-hydroxyethyl and 2-hydroxypropyl.

With regard to the compounds described herein, Z is a bond, —C(O)—, —C═NOR¹², C═C(R¹⁴)₂, —C(R¹)₂—, —O—, —N(R¹⁰)— or —S(O)ₙ—. In one embodiment, Z is a bond. In another embodiment, Z is —C(R¹)₂—. In another embodiment, Z is —O—. In still another embodiment, Z is —N(R¹⁰)—. In another embodiment, Z is —S(O)—. In another embodiment, Z is —S(O)₂—. In yet another embodiment, Z is —S(O)—. In another embodiment, Z is —S—. In another embodiment, Z is —CH₂—.

With regard to the compounds described herein, each occurrence of R¹ is independently hydrogen, C₁-C₆alkyl, cycloalkyl, halogen, haloalkyl or —OR⁷, wherein OR⁷ is not adjacent to —N—W—R³. In one embodiment, each occurrence of R¹ is hydrogen, halogen or —OH. In another embodiment, each occurrence of R¹ is hydrogen. In still another embodiment, at least one occurrence of R¹ is —OH. In another embodiment, at least one occurrence of R¹ is halogen. In another embodiment, at least one occurrence of R¹ is fluorine. In yet another embodiment, R¹ is methyl. In still yet another embodiment, at least one occurrence of R¹ is fluoromethyl, difluoromethyl or trifluoromethyl.

With regard to the compounds described herein, each occurrence of R² is independently hydrogen or C₁-C₆alkyl. In another embodiment, at least one occurrence of R² is hydrogen or C₁-C₆alkyl. In another embodiment, at least one occurrence of R² is hydrogen. In another embodiment, each occurrence of R² is hydrogen.

With regard to the compounds described herein, R³ is C₁-C₆alkyl, C₂-C₆alkenyl, C₂-C₆alkynyl, haloalkyl, —(C₁-C₆alky)ₜ-cycloalkyl, —(C₁-C₆alkyl)ₜ-heterocycloalkyl, —(C₁-C₆alkyl)ₜ-aryl or —(C₁-C₆alkyl)ₜ-heteroaryl, wherein the cycloalkyl, heterocycloalkyl, aryl or heteroaryl group can be unsubstituted or substituted with one or more substituents each independently selected from R⁹. In one embodiment, R³ is C₁-C₆alkyl, C₂-C₆alkenyl, C₂-C₆alkynyl, haloalkyl, —(C₁-C₆alkyl)ₜ-cycloalkyl, —(C₁-C₆alkyl)ₜ-heterocycloalkyl or —(C₁-C₆alkyl)ₜ-heteroaryl, wherein the cycloalkyl, heterocycloalkyl or heteroaryl group can be unsubstituted or substituted with one or more substituents each independently selected from R⁹. In one embodiment of the compounds described herein, R³ is C₁-C₆alkyl, haloalkyl, heteroaryl or cycloalkyl. In one embodiment, R³ is C₁-C₆alkyl. In another embodiment, R³ is a linear alkyl group. In another embodiment, R³ is a branched alkyl group. In still another embodiment, R³ is methyl. In another embodiment, R³ is ethyl. In another embodiment, R³ is isopropyl. In a further embodiment, R³ is t-butyl. In another embodiment, R³ is alkenyl. In another embodiment, R³ is alkynyl. In one embodiment, R³ is haloalkyl. In another embodiment, R³ is —CH₂CF₃. In another embodiment, R³ is —CH(CF₃)₂. In one embodiment, R³ is cycloalkyl.

in another embodiment, R³ is cycloalkyl, which can be optionally substituted with up to 4 substituents, each independently selected from C₁-C₆alkyl and halogen. In another embodiment, R³ is cycloalkyl, which can be optionally substituted with up to 4 substituents, each independently selected from methyl and fluorine. In certain embodiments, R³ is substituted with one substituent. In other embodiments, R³ is substituted with two substituents. In still another embodiment, R³ is cyclopropyl. In another embodiment, R³ is cyclobutyl. In another embodiment, R³ is 1-methylcyclopropyl. In yet another embodiment, R³ is 1-methylcyclobutyl. In one embodiment, R³ is cyclopentyl. In another embodiment, R³ is cyclohexyl.

In one embodiment, R³ is isopropyl, t-butyl, difluoromethyl, trifluoromethyl, 2,2-difluoroethyl, 2,2,2-trifluoroethyl, cyclopropyl or cyclobutyl, wherein the difluormethyl, cyclopropyl or cyclobutyl group can be optionally substituted with C₁-C₆alkyl, trifluoromethyl or halogen.

With regard to the compounds described herein, each occurrence of R⁴ is independently hydrogen or C₁-C₆alkyl. In one embodiment, R⁴ is hydrogen. In another embodiment, R⁴ is C₁-C₆alkyl.

With regard to the compounds described herein, each occurrence of R⁷ is independently hydrogen or C₁-C₆alkyl. In one embodiment, R⁷ is hydrogen. In another embodiment, R⁷ is C₁-C₆alkyl. For example in one embodiment, R⁷ is hydrogen and each occurrence of R¹ and R² is hydrogen.

With regard to the compounds described herein, R⁸ is hydrogen, halogen, C₁-C₆alkyl or cycloalkyl. In one embodiment, R⁸ is hydrogen. In another embodiment, R⁸ is halogen. In another embodiment, R⁸ is C₁-C₆alkyl. In still another embodiment, R⁸ is cycloalkyl.

With regard to the embodiments of the compounds described herein, R⁹ represents C₁-C₆alkyl, C₂-C₆alkenyl, C₂-C₆alkynyl, halogen, haloalkyl, —CN, —NO₂, —O—(C₁-C₆alkyl)ₜ-R¹³, —S—(C₁-C₆alkyl)ₜ-R¹³, —N(R¹³)—(C₁-C₆alkyl)ₜ-R¹³, —(C₁-C₆alkyl)ₜ-R¹³, —C(O)—(C₁-C₆alkyl)ₜ-R¹³, —C(O)O—(C₁-C₆alkyl)ₜ-R¹³, —N(R⁷)C(O)—(C₁-C₆alkyl)ₜ-R¹³, —C(O)N(R⁷)—(C₁-C₆alkyl)ₜ-R¹³, —OC(O)—(C₁-C₆alkyl)ₜ-R¹³, —N(R⁷)C(O)N(R⁷)—(C₁-C₆alkyl)ₜ-R¹³, —N(R⁷)C(O)O—(C₁-C₆alkyl)ₜ-R¹³, —S(O)—(C₁-C₆alkyl)ₜ-R¹³ or —S(O)₂(C₁-C₆alkyl)ₜ-R¹³. In certain embodiments, R⁹ is halogen, wherein the halogen is fluorine or chlorine. In other embodiments, R⁹ is CN. In yet other embodiments, R⁹ is —S(O)₂(C₁-C₆alkyl)ₜ-R¹³, wherein —S(O)₂(C₁-C₆alkyl)ₜ-R¹³ is —S(O)₂Me. In yet other embodiments, R⁹ is —C(O)—(C₁-C₆alkyl)ₜ-R¹³, wherein —C(O)—(C₁-C₆alkyl)ₜ-R¹³ is —C(O)cyclopropyl.

In regard to the compounds described herein, R¹⁰ is hydrogen, C₁-C₆alkyl, aryl, or —C(O)OR⁴. In certain embodiments of the compounds described herein, each occurrence of R¹⁰ is hydrogen. In other embodiments of the compounds described herein, at least one occurrence of R¹⁰ is hydrogen. In other embodiments of the compounds described herein, at least one occurrence of R¹⁰ is aryl. In other embodiments of the compounds described herein, at least one occurrence of R¹⁰ is C₁-C₆alkyl. In other embodiments of the compounds described herein, at least one occurrence of R¹⁰ is C(O)OR⁴.

In regard to the compounds described herein, each occurrence of R¹¹ is independently hydrogen, C₁-C₆alkyl, aryl, cycloalkyl, heterocycloalkyl, heteroaryl, —N(R⁷)₂ or halogen. In some embodiments of the compounds described herein, each occurrence of R¹¹ is hydrogen. In other embodiments of the compounds described herein, at least one occurrence of R¹¹ is C₁-C₆alkyl. In other embodiments of the compounds described herein, at least one occurrence of R¹¹ is aryl or heteroaryl. In other embodiments of the compounds described herein, at least one occurrence of R¹¹ is cycloalkyl or heterocycloalkyl. In other embodiments of the compounds described herein, at least one occurrence of R¹¹ is halogen. In other embodiments of the compounds described herein, at least one occurrence of R¹¹ is —N(R⁷)₂.

With regard to the compounds described herein, at every occurrence R¹² is independently hydrogen, C₁-C₆alkyl or aryl. In certain embodiments, at every occurrence R¹² is hydrogen. In other embodiment, in at least one occurrence R¹² is C₁-C₆alkyl. In still other embodiments, in at least one occurrence R¹² is aryl.

With regard to the embodiments of the compounds described herein, R¹³ is independently hydrogen, hydroxyl, haloalkyl, aryl, cycloalkyl, —COOC₁-C₆alkyl, —OC₁-C₆alkyl or heteroaryl. In one embodiment, R¹³ is hydrogen. In another embodiment, R¹³ is haloalkyl. In yet another embodiment, R¹³ is aryl. In one embodiment, R¹³ is cycloalkyl. In another embodiment, R¹³ is heteroaryl. In yet another embodiment, R¹³ is hydroxyl. In still another embodiment R¹³ is —COOC₁-C₆alkyl. In yet another embodiment, R¹³ is —OC₁-C₆alkyl With regard to the compounds described herein, —(C₁-C₆alkyl)ₜ-R¹³, wherein t is 1 or 2, means at least one hydrogen on the —(C₁-C₆alkyl) or —(C₁-C₆alkyl)₂ is replaced with a substituent selected from R¹³. For example, —O—(C₁-C₆alkyl)ₜ-R¹³, wherein t is 1 and C₁-C₆alkyl is methyl and R¹³ is trifluoromethyl means one hydrogen on the methyl is substituted with the trifluoromethlyl i.e. trifluoroethyl. In another example, —O—(C₁-C₆alkyl)ₜ-R¹³, wherein t is 1 and C₁-C₆alkyl is methyl and R¹³ is hydrogen means one hydrogen on the methyl is substituted with the R¹³ hydrogen resulting in methyl.

In certain embodiments, at every occurrence R¹⁴ is hydrogen. In other embodiment, in at least one occurrence R¹⁴ is C₁-C₆alkyl. In still other embodiment, in at least one occurrence R¹⁴ is aryl. In still another embodiment, both R¹⁴ groups, and the carbon atom to which they are attached, combine to form a cycloalkyl or heterocycloalkyl group.

With regard to the compounds described herein, Ar is aryl, heteroaryl, heterocycloalkyl or cycloalkyl, any of which can be unsubstituted or substituted with one or more substituents each independently selected from R⁹. In one embodiment, Ar is aryl or heteroaryl. In another embodiment, Ar is aryl. In another embodiment, Ar is heteroaryl. In another embodiment, Ar is pyridyl. In still another embodiment, Ar is cycloalkyl. In one embodiment, Ar is phenyl.

In one embodiment, Ar is substituted with R⁹, wherein R⁹ is selected from alkyl, halogen, —CN, cycloalkyl, alkynyl, heteroaryl, —OC₁-C₆alkyl, —COC₁-C₆alkyl, —COOC₁-C₆alkyl, COcycloalkyl, C₁-C₆alkyl-OH, —S(O₂)-alkyl, or —S(O₂)-cycloalkyl. In another embodiment, Ar is substituted with R⁹, wherein R⁹ is selected from methyl, fluorine, chlorine, —CN, cyclopropyl, cyclobutyl, —C═CH, —C≡C—CH₃, imidazolyl, triazolyl, pyrazolyl, isoxazolyl, thiazolyl, oxazolyl, —S(O)₂CH₃, or —S(O)₂-cyclopropyl.

In another embodiment, Ar is phenyl, which is substituted with 1 or 2 groups, each independently selected from halogen, —CN or —S(O)₂-alkyl.

In still another embodiment, Ar is heteroaryl, which is substituted with 1 or 2 groups, each independently selected from alkyl and heteroaryl.

In one embodiment, Ar is:

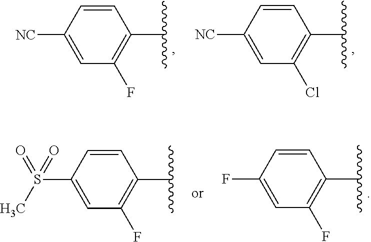

In another embodiment, Ar is:

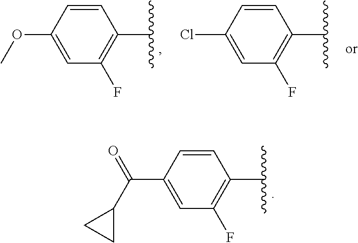

In still another embodiment, Ar is:

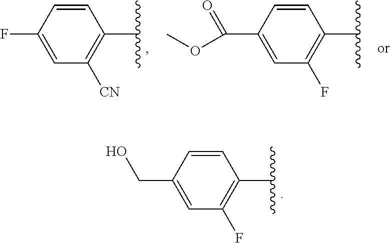

In still another embodiment, Ar is:

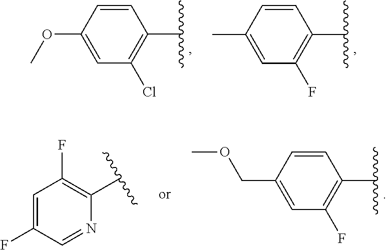

In still another embodiment, Ar is:

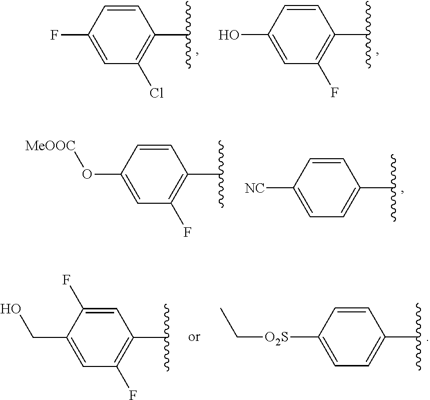

In yet another embodiment, Ar is:

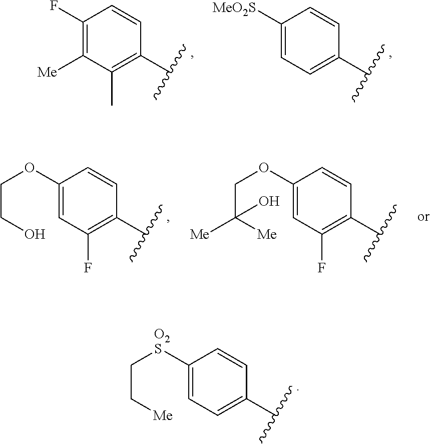

In yet another embodiment Ar is:

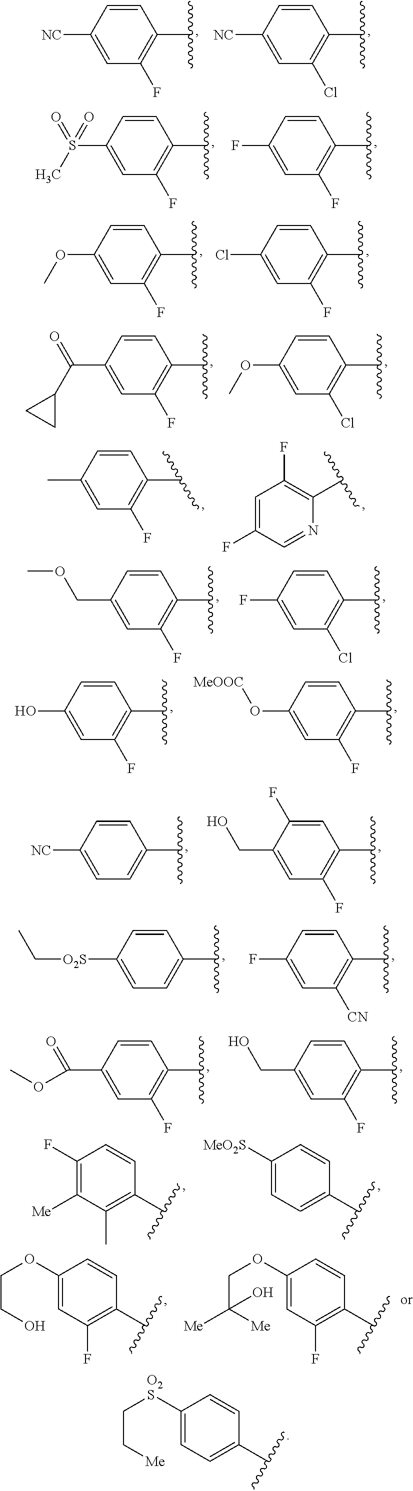

In one embodiment of the compounds A is —O— and W is —C(O)O— or a bond.

In one embodiment, W is —C(O)O— and R³ is C₁-C₆alkyl, cycloalkyl or haloalkyl.

In another embodiment, W is —C(O)O— and R³ is cyclopropyl, cyclobutyl, isopropyl, t-butyl, —CF₃ or —CH(CF₃)₂.

In one embodiment, W is a bond and R³ is heteroaryl or cycloalkyl.

In another embodiment, W is a bond and R³ is heteroaryl.

In another embodiment, W is a bond and R³ is pyrimidine.

In one embodiment, p and u are each 0.

In one embodiment, p and u are each 1.

In another embodiment, p and u are each 0, and r and s are each 1.

In another embodiment, q, r and s are each 1, p and u are each 0 and Z is —O—.

In one embodiment, q, r and s are each 1; p and u are each 0; Z is —O—; and W is —C(O)O— or a bond.

In another embodiment, q, r and s are each 1; p and u are each 0; Z is —O—; W is —C(O)O— or a bond; and A is —O—.

In another embodiment, q, r and s are each 1; p and u are each 0; Z is —O—; W is —C(O)O— or a bond; A is —O—; and R³ is C₁-C₆alkyl or cycloalkyl.

In yet another embodiment, q, r and s are each 1; p and n are each 0; Z is —O—; W is —C(O)O— or a bond; A is —O—; and R⁸ is hydrogen.

In a further embodiment, q, r and s are each 1; p and u are each 0; Z is —O—; W is —C(O)O— or a bond; A is —O—; R³ is C₁-C₆alkyl or cycloalkyl; and R⁸ is hydrogen.

In one embodiment, q, r and s are each 1; p and u are each 0; Z is —O—; and W is —C(O)O—.

In another embodiment, q, r and s are each 1; p and u are each 0; Z is —O—; W is —C(O)O—; and A is —O—.

In another embodiment, q, r and s are each 1; p and u are each 0; Z is —O—; W is —C(O)O—;

A is —O—; and R³ is C₁-C₆alkyl or cycloalkyl.

In yet another embodiment, q, r and s are each 1; p and u are each 0; Z is —O—; W is —C(O)O—; A is —O—; and R⁸ is hydrogen.

In a further embodiment, q, r and s are each 1; p and u are each 0; Z is —O—; W is —C(O)O—; A is —O—; R³ is C₁-C₆alkyl or cycloalkyl; and R⁸ is hydrogen.

In one embodiment, q, r and s are each 1; p and u are each 0; Z is —O—; and W is a bond.

In another embodiment, q, r and s are each 1; p and u are each 0; Z is —O—; W is a bond; and A is —O—.

In another embodiment, q, r and s are each 1; p and u are each 0; Z is —O—; W is a bond; A is —O—; and R³ is C₁-C₆alkyl or cycloalkyl.

In yet another embodiment, q, r and s are each 1; p and u are each 0; Z is —O—; W is a bond; A is —O—; and R⁸ is hydrogen.

In a further embodiment, q, r and s are each 1; p and u are each 0; Z is —O—; W is a bond; A is —O—; R³ is C₁-C₆alkyl or cycloalkyl; and R⁸ is hydrogen.

Also described herein are compounds of formula Ia:

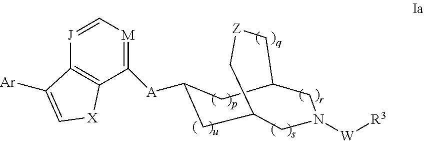

or pharmaceutically acceptable salt thereof, wherein J and M are independently —N— or —C(R¹¹)—, wherein R₁₁ is hydrogen, C₁-C₆alkyl, aryl, cycloalkyl, heterocycloalkyl, heteroaryl, —N(R⁷)₂ or halogen, wherein one of J or M must be —N—; W is a bond, C₁-C₆alkyl, —C(O)—, —C(O)O—, —C(S)O—, or —SO₂; X is —O—, —S—, —NH, —N(C₁-C₆alkyl), —N(cycloalkyl) or —N-(hydroxyaryl); A is —O—, —S—, —NH or —N(C₁-C₆allyl); Z is a bond, —O—, —S(O)ₙ, NH, N(C₁-C₆alkyl), N-cycloalkyl or —C(O)—, OH; Ar is phenyl or heteroaryl, wherein the phenyl or heteroaryl is unsubstituted or substituted with one or more substituents each independently selected from the group consisting of C₁-C₆alkyl, cycloalkyl, haloalkyl, halogen, —O—C₁-C₆, —OH, —C₁-C₆alkyl-OH, —CN, —COC₁-C₆alkyl, CO-cycloalkyl, —C(O)O—C₁-C₆alkyl, S(O)₂C₁-C₆alkyl, S(O)₂cycloalkyl, and heteroaryl; R³ is C₁-C₆alkyl, cycloalkyl, haloalkyl or heteroaryl, wherein the cycloalkyl or heteroaryl are unsubstituted or substituted with one or more substituents each independently selected from the group consisting of C₁-C₆alkyl, halogen, OC₁-C₆alkyl, CO(O)C₁-C₆alkyl, cycloalkyl, haloalkyl and —CN; R⁷ is hydrogen, C₁-C₆alkyl or halogen; n=0, 1 or 2; p=0, 1 or 2; q=0, 1 or 2; r=0, 1 or 2, wherein when r=0, then s is non-zero; s=0, 1 or 2; u=0, 1 or 2.

In regard to the compounds described herein, J and M are independently —N— or —C(R¹¹)—. In one embodiment, J is —C(R¹¹)—. In another embodiment, J is —N—. In another embodiment, J is —CH— or —N—. In still another embodiment, J is —CH—. In one embodiment, M is —C(R¹¹)—. In another embodiment, M is —N—. In another embodiment, M is —CH— or —N—. In still another embodiment, M is —CH—. In yet another embodiment of the compounds described herein, J and M are each —N—.

In regard to the compounds described herein, X is —O—, —S—, —NH, —N(C₁-C₆alkyl), —N(cycloalkyl) or —N(hydroxyaryl). In one embodiment of the compounds described herein, X is —O—. In another embodiment, X is —S—. In yet another embodiment, X is —NH. In still another embodiment, X is —N(C₁-C₆alkyl). In one embodiment, X is —S—, —NH— or O. In yet another embodiment, X is —N(cycloalkyl). Examples of cycloalkyl include, but are not limited to, cyclopropyl, cylcobutyl, cyclopentyl and cylcohexyl. In still another embodiment, X is —N(hydroxyaryl). Examples include hydroxyphenyl.

In regard to the compounds described herein W is a bond, C₁-C₆alkyl, —C(O)—, —C(O)—O—, —S(O)₂—, —S(O)₂—N(R¹⁰)—, C(S)O or —C(O)N(R¹⁰)—. In one embodiment, W is —C(O)O—, —S(O)₂—, —C(O)— or C₁-C₆alkyl. In another embodiment, W is —C(O)O— or —S(O)₂—. In still another embodiment, W is —C(O)O— or —C(O)—. In another embodiment, W is a bond. In still another embodiment, W is C₁-C₆alkyl. In another embodiment, W is —C(O)—. In yet another embodiment, W is —S(O)₂—. In another embodiment, W is —CH₂—. In another embodiment, W is —C(O)O—. In yet another embodiment, W is —S(O)₂N(R¹⁰). In a further embodiment, W is —C(O)N(R¹⁰)—. In an additional embodiment, W is a bond or —C(O)O—.

In certain embodiments of the compounds described herein A is —O—. In other embodiments of the compounds described herein, A is —S—. In still other embodiments of the compounds described herein, A is —NH. In yet other embodiments of the compounds described herein A is —N(C₁-C₆alkyl)-.

With regard to the compounds described herein, Z is bond, —O—, —S(O)ₙ, —NH, —N(C₁-C₆alkyl), N-cycloalkyl or —C(O). In one embodiment, Z is a bond. In another embodiment, Z is —NH. In another embodiment, Z is —O—. In still another embodiment, Z is —N(C₁-C₆alkyl). In another embodiment, Z is —S(O)ₙ—. In another embodiment, Z is —S(O)₂—. In yet another embodiment, Z is —S(O)—. In another embodiment, Z is —S—. In another embodiment, Z is —N-cycloalkyl. In another embodiment, Z is —C(O)—.

In regard to the compounds described herein, Ar is phenyl or heteroaryl, wherein the phenyl or heteroaryl is unsubstituted or substituted with one or more substituents each independently selected from the group consisting of C₁-C₆alkyl, cycloalkyl, haloalkyl, halogen, C₁-C₆alkoxy, —OH, —C₁-C₆alkyl-OH, —CN, COC₁-C₆alkyl, —C(O)O—C₁-C₆alkyl, CO-cycloalkyl, S(O)₂C₁-C₆alkyl, S(O)₂cycloalkyl, and heteroaryl. In one embodiment, Ar is phenyl. In another embodiment, Ar is substituted with 1 to 4 substituents each independently selected from the group consisting of halogen, —CN, —OC₁-C₆alkyl, CO-cycloalkyl, —C(O)O—C₁-C₆alkyl, —C₁-C₆alkyl-OH and S(O)₂C₁-C₆alkyl.

With regard to the compounds described herein, R³ is C₁-C₆alkyl, cycloalkyl, haloalkyl or heteroaryl, wherein the cycloalkyl or heteroaryl are unsubstituted or substituted with one or more substituents each independently selected from the group consisting of C₁-C₆alkyl, halogen, OC₁-C₆alkyl, CO(O)C₁-C₆alkyl, cycloalkyl, haloalkyl and —CN. In one embodiment of the compounds described herein, R³ is C₁-C₆alkyl, haloalkyl, heteroaryl or cycloalkyl. In one embodiment, R³ is C₁-C₆alkyl. In another embodiment, R³ is a linear alkyl group. In another embodiment, R³ is a branched alkyl group. In still another embodiment, R³ is methyl. In another embodiment, R³ is ethyl. In another embodiment, R³ is isopropyl. In a further embodiment, R³ is t-butyl. In another embodiment, R³ is alkenyl. In another embodiment, R³ is alkynyl. In one embodiment, R³ is haloalkyl. In another embodiment, R³ is —CF₃. In another embodiment, R³ is —CF₂CF₃. In another embodiment, R³ is —CH(CF₃)₂. In one embodiment, R³ is cycloalkyl. In still another embodiment, R³ is cycloalkyl, wherein the cycloalkyl is substituted with a C₁-C₆alkyl. In yet another embodiment, R³ is heteroaryl, wherein the heteroaryl is substituted with one or more substituents each independently selected from the group consisting of halogen and C₁-C₆alkyl. In one additional embodiment, R³ is heteroaryl, wherein the heteroaryl is pyrimidine.

In another embodiment, R³ is cycloalkyl, which can be optionally substituted with up to 4 substituents, each independently selected from C₁-C₆alkyl and halogen. In certain embodiments, R³ is substituted with one substituent. In other embodiments, R³ is substituted with two substituents. In another embodiment, R³ is cycloalkyl, which can be optionally substituted with up to 4 substituents, each independently selected from methyl and fluorine. In still another embodiment, R³ is cyclopropyl. In another embodiment, R³ is cyclobutyl. In another embodiment, R³ is 1-methylcyclopropyl. In yet another embodiment, R³ is 1-methylcyclobutyl. In one embodiment, R³ is cyclopentyl. In another embodiment, R³ is cyclohexyl.

In one embodiment, R³ is isopropyl, t-butyl, trifluoromethyl, cyclopropyl or cyclobutyl, wherein the cyclopropyl or cyclobutyl group can be optionally substituted with up to 4 substituents, each independently selected from C₁-C₆alkyl and halogen.

With regard to the compounds described herein, p=0, 1 or 2; q=0, 1 or 2; r=0, 1 or 2, wherein when r=0, then q is non-zero; s 0, 1 or 2; u=0, 1 or 2. In one embodiment p is 0. In another embodiment p is 1. In still another embodiment, p is 2. In one embodiment q is 0. In another embodiment q is 1. In still another embodiment, q is 2. In one embodiment r is 0, wherein when r=0, then q is non-zero. In another embodiment r is 1. In still another embodiment, r is 2. In one embodiment s is 0. In another embodiment s is 1. In still another embodiment, s is 2. In one embodiment u is 0. In another embodiment u is 1. In still another embodiment, u is 2. For example in one embodiment, p and u are 0 and q, r and s are 1.

Also described herein are compounds of formula Ib

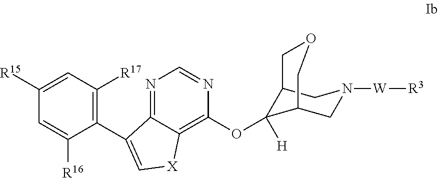

or pharmaceutically acceptable salt thereof, wherein W is a bond, C₁-C₆alkyl, —C(O)—, —C(O)O—, —C(S)O— or —SO₂; X is —O—, —S—, —NH or —N(C₁-C₆alkyl); R³ is C₁-C₆alkyl, cycloalkyl, haloalkyl or heteroaryl, wherein cycloalkyl is unsubstituted or substituted with 1 to 3 substituents each independently selected from the group consisting of C₁-C₆alkyl, halogen, OC₁-C₆alkyl, and CO(O)C₁-C₆alkyl, wherein heteroaryl is unsubstituted or substituted with 1 to 3 substituents each independently selected from the group consisting of C₁-C₆alkyl, cycloalkyl, haloalkyl, halogen, —CN and OC₁-C₆alkyl, and wherein haloalkyl is unsubstituted or substituted with haloalkyl; R¹⁵ is C₁-C₆alkyl, cycloalkyl, haloalkyl, halogen, —O—C₁-C₆alkyl, —CN, —COC₁-C₆alkyl, —C₁-C₆alkyl-OH, —C(O)O—C₁-C₆alkyl, —COcycloalkyl, —S(O)₂C₁-C₆alkyl, —S(O)₂cycloalkyl, or heteroaryl; R¹⁶ is hydrogen, CN or halogen; and R¹⁷ is hydrogen or halogen.

In regard to the compounds described herein, X is —O—, —S—, —NH or —N(C₁-C₆alkyl). In one embodiment of the compounds described herein, X is —O—. In another embodiment, X is —S—, In yet another embodiment, X is —NH. In still another embodiment, X is —N(C₁-C₆alkyl). In one embodiment, X is —S—, —NH— or O.

In regard to the compounds described herein W is a bond, C₁-C₆alkyl, —C(O)—, —C(O)—O—, —C(O)—N(R¹⁰)—, —S(O)₂—N(R¹⁰)—, C(S)O or —S(O)₂—. In one embodiment, W is —C(O)O—, —S(O)₂—, —C(O)— or C₁-C₆alkyl. In another embodiment, W is —C(O)O— or —S(O)₂—. In still another embodiment, W is —C(O)O— or —C(O)—. In another embodiment, W is a bond. In still another embodiment, W is C₁-C₆alkyl. In another embodiment, W is —C(O)—. In still another embodiment, W is —C(O)O—. In yet another embodiment, W is —S(O)₂—. In another embodiment, W is —CH₂—. In an additional embodiment, W is a bond or —C(O)O—.

With regard to the compounds described herein, R³ is C₁-C₆alkyl, cycloalkyl, haloalkyl or heteroaryl, wherein cycloalkyl is unsubstituted or substituted with 1 to 3 substituents each independently selected from the group consisting of C₁-C₆alkyl, halogen, OC₁-C₆alkyl, and CO(O)C₁-C₆alkyl, wherein heteroaryl is unsubstituted or substituted with 1 to 3 substituents each independently selected from the group consisting of C₁-C₆alkyl, cycloalkyl, haloalkyl, halogen, —CN and OC₁-C₆alkyl. In one embodiment, R³ is C₁-C₆alkyl. In another embodiment, R³ is a linear alkyl group. In another embodiment, R³ is a branched alkyl group. In still another embodiment, R³ is methyl. In another embodiment, R³ is ethyl. In another embodiment, R³ is isopropyl. In a further embodiment, R³ is t-butyl. In one embodiment, R³ is haloalkyl. In another embodiment, R³ is —CF₃. In another embodiment, R³ is —CF₂CF₃. In another embodiment, R³ is —CH(CF₃)₂. In one embodiment, R³ is cycloalkyl. In still another embodiment, R³ is cycloalkyl, wherein the cycloalkyl is substituted with C₁-C₆alkyl, halogen, OC₁-C₆alkyl or CO(O)C₁-C₆alkyl. In yet another embodiment, R³ is heteroaryl, wherein the heteroaryl is substituted with one or more substituents each independently selected from the group consisting of C₁-C₆alkyl, cycloalkyl, haloalkyl, halogen, —CN and OC₁-C₆alkyl. In one additional embodiment, R³ is heteroaryl, wherein the heteroaryl is pyrimidine.

In another embodiment, R³ is cycloalkyl, which can be optionally substituted with up to 4 substituents, each independently selected from C₁-C₆alkyl and halogen. In certain embodiments, R³ is substituted with one substituent. In other embodiments, R³ is substituted with two substituents. In another embodiment, R³ is cycloalkyl, which can be optionally substituted with up to 4 substituents, each independently selected from methyl and fluorine. In still another embodiment, R³ is cyclopropyl. In another embodiment, R³ is cyclobutyl. In another embodiment, R³ is 1-methylcyclopropyl. In yet another embodiment, R³ is 1-methylcyclobutyl. In one embodiment, R³ is cyclopentyl. In another embodiment, R³ is cyclohexyl.

In one embodiment, R³ is isopropyl, t-butyl, trifluoromethyl, cyclopropyl or cyclobutyl, wherein a cyclopropyl or cyclobutyl group can be optionally substituted with up to 4 substituents, each independently selected from C₁-C₆alkyl and halogen. In another embodiment, R³ is C₁-C₆alkyl, C₃-C₆cycloalkyl or heteroaryl, wherein the C₃-C₆cycloalkyl and heteroaryl are substituted with one or more substituents each independently selected from the group consisting of halogen and C₁-C₆alkyl. In another embodiment, R³ is haloalkyl. In one example R³ is —CF₂CF₃.

With regard to the compounds of formula Ib, R¹⁵ is C₁-C₆alkyl, cycloalkyl, haloalkyl, halogen, —O—C₁-C₆alkyl, —CN, —COC₁-C₆alkyl, —COcycloalkyl, —S(O)₂C₁-C₆alkyl, —S(O)₂cycloalkyl, or heteroaryl. In certain embodiments, R¹⁵ is halogen, —CN or —S(O)₂Me. In one embodiment, R¹⁵ is halogen, wherein the halogen is fluorine or chlorine.

With regard to the compounds of formula Ib, R¹⁶ is hydrogen, CN or halogen. In certain embodiments of the compounds described herein R¹⁶ is halogen, wherein the halogen is fluorine or chlorine. In other embodiments, R¹⁶ is CN. In still other embodiments, R¹⁶ is hydrogen.

With regard to the compounds of formula Ib, R¹⁷ is halogen or hydrogen. In one embodiment, R¹⁷ is halogen. In certain embodiments of the compounds described herein R¹⁶ is halogen, wherein the halogen is fluorine or chlorine. In another embodiment R¹⁷ is hydrogen.

Examples of compounds described herein include, but are not limited to, a compound or a pharmaceutically acceptable salt thereof, selected from the group consisting of:

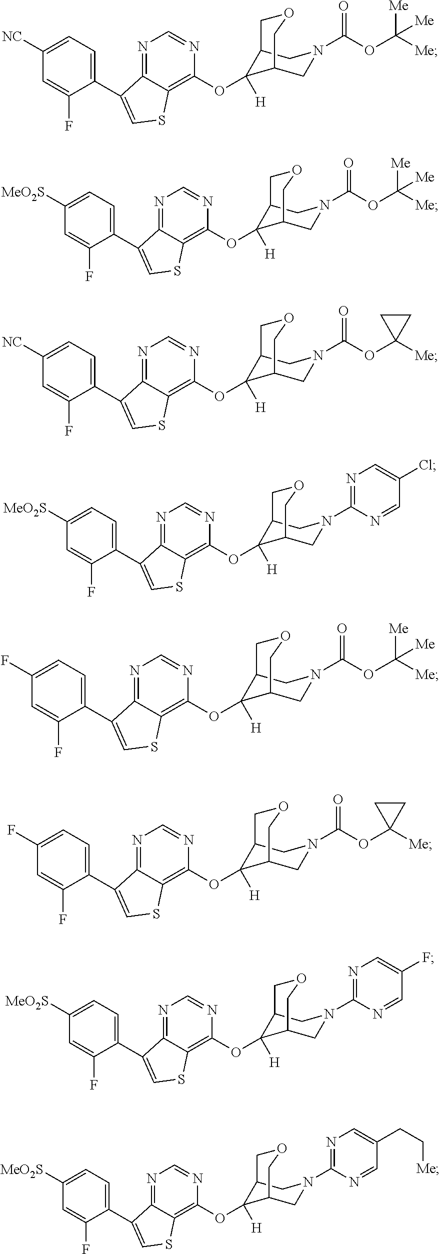

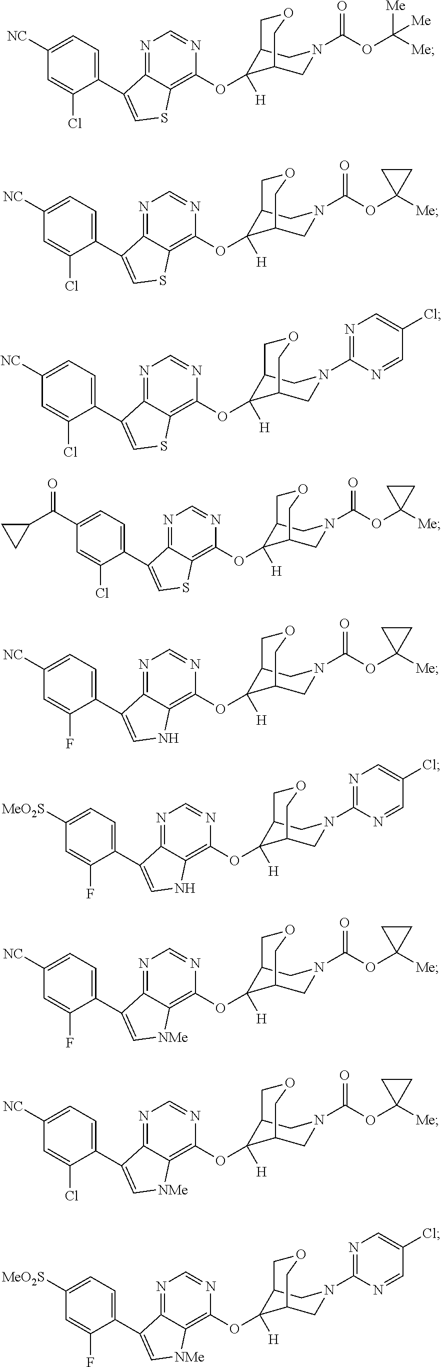

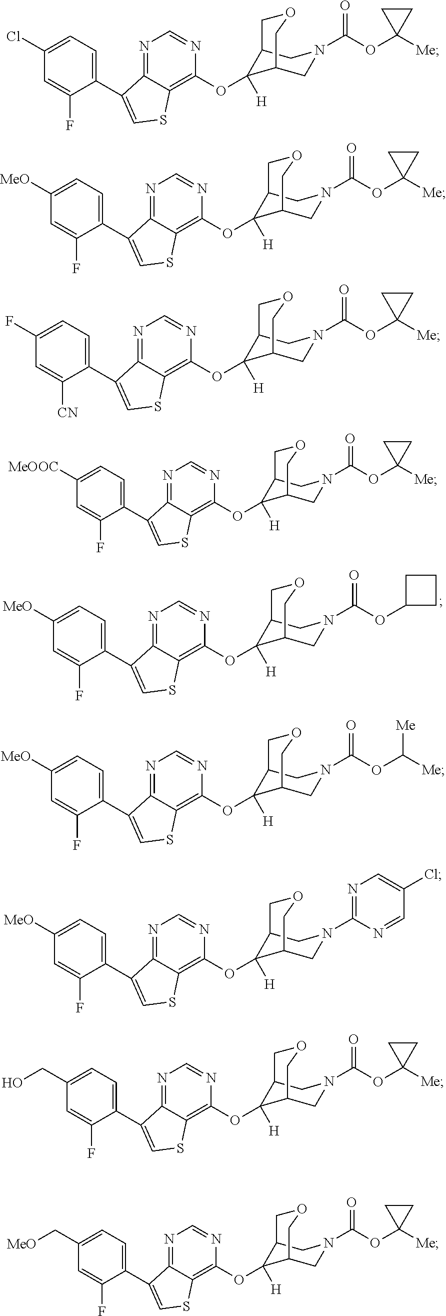

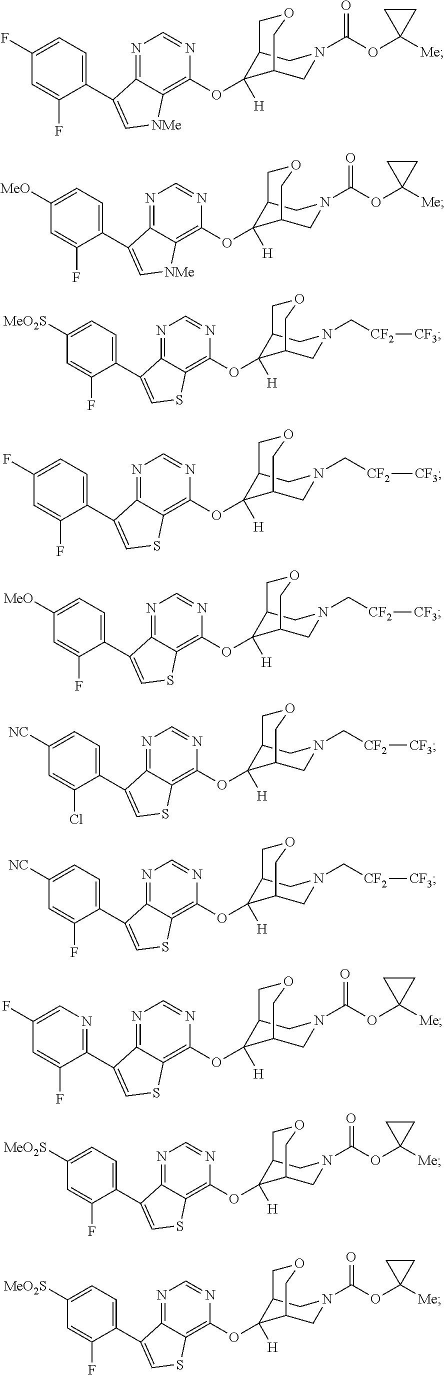

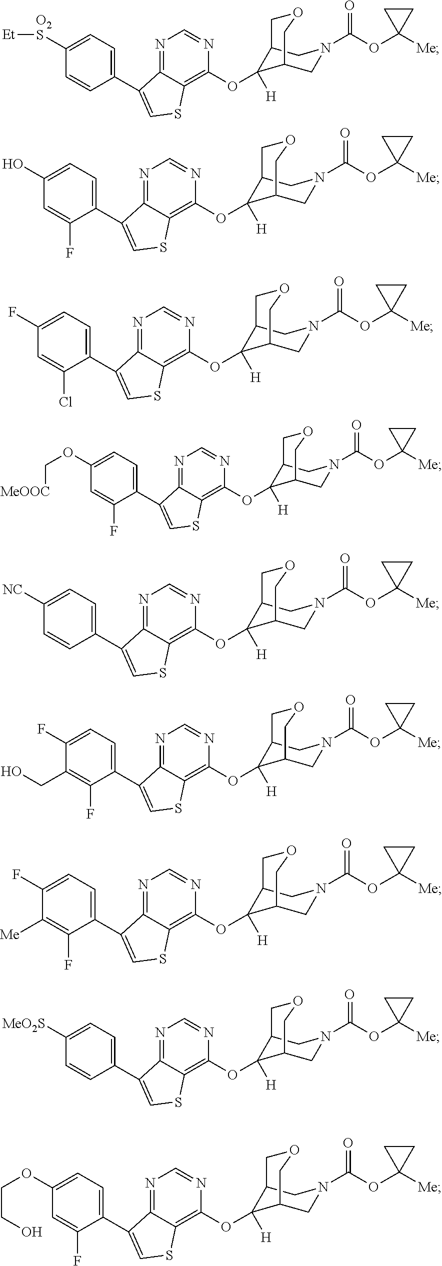

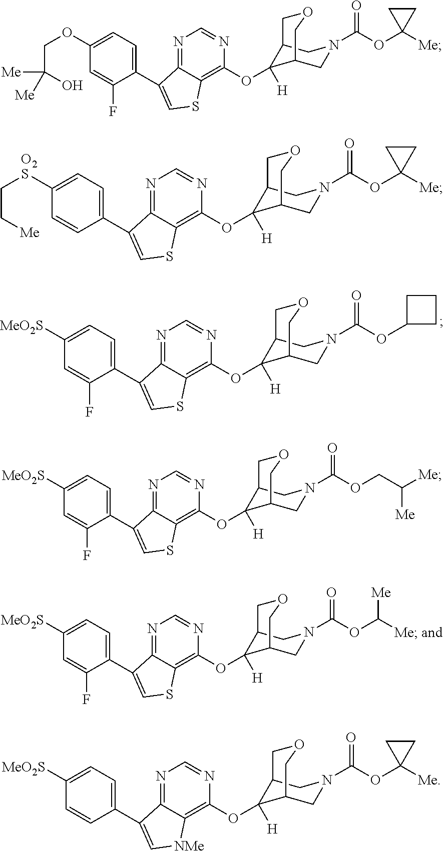

### DEFINITIONS

As used above, and throughout this disclosure, the following terms, unless otherwise indicated, shall be understood to have the following meanings:

A “subject” is a human or non-human mammal. In one embodiment, a subject is a human. In another embodiment, a subject is a non-human mammal, including, but not limited to, a monkey, dog, baboon, rhesus, mouse, rat, horse, cat or rabbit. In another embodiment, a subject is a companion animal, including but not limited to a dog, cat, rabbit, horse or ferret. In one embodiment, a subject is a dog. In another embodiment, a subject is a cat.

The term “obesity” as used herein, refers to a patient being overweight and having a body mass index (BMI) of 25 or greater. In one embodiment, an obese patient has a BMI of 25 or greater. In another embodiment, an obese patient has a BMI from 25 to 30. In another embodiment, an obese patient has a BMI greater than 30. In still another embodiment, an obese patient has a BMI greater than 40.

The term “obesity-related disorder” as used herein refers to: (i) disorders which result from a patient having a BMI of 25 or greater; and (ii) eating disorders and other disorders associated with excessive food intake. Non-limiting examples of an obesity-related disorder include edema, shortness of breath, sleep apnea, skin disorders and high blood pressure. The term “metabolic syndrome” as used herein, refers to a set of risk factors that make a patient more succeptible to cardiovascular disease and/or type 2 diabetes. A patient is said to have metabolic syndrome if the patient simultaneously has three or more of the following five risk factors:
1) central/abdominal obesity as measured by a waist circumference of greater than 40 inches in a male and greater than 35 inches in a female;
2) a fasting triglyceride level of greater than or equal to 150 mg/dL;
3) an HDL cholesterol level in a male of less than 40 mg/dL or in a female of less than 50 mg/dL;
4) blood pressure greater than or equal to 130/85 mm Hg; and
5) a fasting glucose level of greater than or equal to 110 mg/dL.

The term “effective amount” as used herein, refers to an amount of any of the compounds described herein and/or an additional therapeutic agent, or a composition thereof that is effective in producing the desired therapeutic, ameliorative, inhibitory or preventative effect when administered to a patient suffering from a condition. In the combination therapies of the present invention, an effective amount can refer to each individual agent or to the combination as a whole, wherein the amounts of all agents administered are together effective, but wherein the component agent of the combination may not be present individually in an effective amount.

The term “alkyl,” as used herein, refers to an aliphatic hydrocarbon group which may be straight or branched and unless otherwise specified, contains from about 1 to about 20 carbon atoms. In one embodiment, an alkyl group contains from about 1 to about 12 carbon atoms. In another embodiment, an alkyl group contains from about 1 to about 6 carbon atoms. For example, C₁-C₆alkyl is an alkyl containing 1-6 carbon atoms. Non-limiting examples of alkyl groups include methyl, ethyl, n-propyl, isopropyl, n-butyl, see-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halogen, alkenyl, alkynyl, aryl, cycloalkyl, —CN, —OH, —O-alkyl, —O-aryl, -alkyl-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkyl group is unsubstituted. In another embodiment, an alkyl group is linear. In another embodiment, an alkyl group is branched.

The term “alkenyl,” as used herein, refers to an aliphatic hydrocarbon group containing at least one carbon-carbon double bond and may be straight or branched and unless otherwise specified, contains from about 2 to about 15 carbon atoms. In one embodiment, an alkenyl group contains from about 2 to about 12 carbon atoms. In another embodiment, an alkenyl group contains from about 2 to about 6 carbon atoms. For example, C₂-C₆alkenyl is an alkenyl containing 2-6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halogen, alkenyl, alkynyl, aryl, cycloalkyl, —CN, —OH, —O-alkyl, —O-aryl, -alkyl-C-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkenyl group is unsubstituted.

The term “alkynyl,” as used herein, refers to an aliphatic hydrocarbon group containing at least one carbon-carbon triple bond and which may be straight or branched and contains from about 2 to about 15 carbon atoms. In one embodiment, an alkynyl group contains from about 2 to about 12 carbon atoms. In another embodiment, an alkynyl group contains from about 2 to about 6 carbon atoms. For example, C₂-C₆alkynyl is an alkynyl containing 2-6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of halogen, alkenyl, alkynyl, aryl, cycloalkyl, —CN, —OH, —O-alkyl, —O-aryl, -alkyl-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkynyl group is unsubstituted.

The term “aryl,” as used herein, refers to an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. Non-limiting examples of aryl groups include phenyl and naphthyl. In one embodiment, an aryl group is unsubstituted. In another embodiment, an aryl group is phenyl.

The term “C₃-C₁₀cycloalkyl” means a monocyclic or polycyclic, saturated or partially-unsaturated carbocyclic group having from 3 to 10 carbon atoms, for example, cyclopropyl, cyclobutenyl, cyclopentyl, cyclohexyl, cycloheptyl, cyclooctyl, cyclononyl, cyclodecyl, bicyclohexyl, bicyclodecyl, bicyclononyl, tetrahydronaphthyl, decahydronaphthyl, indanyl and adamantyl.

The term “cycloalkyl,” as used herein, refers to a non-aromatic, monocyclic or polycyclic, saturated or partially-unsaturated carbocyclic group having from 3 to 10 carbon atoms. In one embodiment, a cycloalkyl contains from about 5 to about 10 ring carbon atoms. In another embodiment, a cycloalkyl contains from about 5 to about 7 ring atoms. The term “cycloalkyl” also encompasses a cycloalkyl group, as defined above, which is fused to an aryl (e.g., benzene) or heteroaryl ring. Non-limiting examples of monocyclic cycloalkyls include cyclopropyl, cyclobutyl, cyclopentyl, cyclohexyl, cycloheptyl, cyclopentenyl, cyclohexenyl, cyclohepta-1,3-dienyl, and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. In one embodiment, a cycloalkyl group is unsubstituted.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. A heteroaryl group can be optionally substituted by one or more “ring system substituents” which may be the same or different, and are as defined herein below. A heteroaryl group is joined via a ring carbon or nitrogen atom, and any nitrogen atom of a heteroaryl can be optionally oxidized to the corresponding N-oxide. The term “heteroaryl” also encompasses a heteroaryl group, as defined above, which is fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, triazolyl, 1,2,4-thiadiazolyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like.

“Cycloheteroalkyl” means mono- or bicyclic or bridged saturated rings containing at least one heteroatom selected from N, S and O, each of said ring having from 3 to 10 atoms in which the point of attachment may be carbon or nitrogen. In one embodiment, a heterocycloalkyl group has from about 5 to about 10 ring atoms. In another embodiment, a heterocycloalkyl group has 5 or 6 ring atoms. There are no adjacent oxygen and/or sulfur atoms present in the ring system. Any —NH group in a heterocycloalkyl ring may exist protected such as, for example, as an —N(BOC), —N(Cbz), —N(Tos) group and the like; such protected heterocycloalkyl groups are considered part of this invention. The term also includes monocyclic heterocycle fused to an aryl or heteroaryl group in which the point of attachment is on the non-aromatic portion. Examples of “cycloheteroalkyl” include tetrahydropyranyl, pyrrolidinyl, piperidinyl, piperazinyl, dioxanyl, imidazolidinyl, 2,3-dihydrofuro(2,3-b)pyridyl, benzoxazinyl, benzoxazolinyl, dihydrophthalazinyl, isoindolinyl, benzoxazepinyl, 5,6-dihydroimidazo[2,1-b]thiazolyl, tetrahydroquinolinyl, morpholinyl, tetrahydroisoquinolinyl, dihydroindolyl, thiomorpholinyl, thiazolidinyl, tetrahydrofuranyl, tetrahydrothiophenyl, lactam, lactone, 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluoro-substituted dihydrofuranyl, 7-oxabicyclo[2.2.1]heptenyl, dihydrothiophenyl, dihydrothiopyranyl, and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils). The term also includes bridged rings such as 5-azabicyclo[2.2.1]heptyl, 2,5-diazabicyclo[2.2.1]heptyl, 2-azabicyclo[2.2.1]heptyl, 7-azabicyclo[2.2.1]heptyl, 2,5-diazabicyclo[2.2.2]octyl, 2-azabicyclo[2.2.2]octyl, and 3-azabicyclo[3.2.2]nonyl, and azabicyclo[2.2.1]heptanyl. The cycloheteroalkyl ring may be substituted on the ring carbons and/or the ring nitrogens. A heterocycloalkyl group can be optionally substituted by one or more “ring system substituents” which may be the same or different, and are as defined herein below. The nitrogen or sulfur atom of the heterocycloalkyl can be optionally oxidized to the corresponding N-oxide, S-oxide or S,S-dioxide. A ring carbon atom of a heterocycloalkyl group may be functionalized as a carbonyl group. Illustrative examples of such a heterocycloalkyl group are:

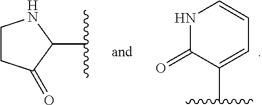 [SMILES: *C1NCCC1=O ; *c1ccc[nH]c1=O]

In one embodiment, a heterocycloalkyl group is unsubstituted. In another embodiment, a heterocycloalkyl group is a 5-membered heterocycloalkyl. In another embodiment, a heterocycloalkyl group is a 6-membered heterocycloalkyl.

“Halogen” means fluorine (—F), chlorine (—Cl), bromine (—Br) or iodine (—I). In one embodiment, halogen refers to fluorine and chlorine.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CF₃, —CH₂Cl and —CCl₃.

The term “alkyl-OH,” as used herein, refers to an alkyl group as defined above, wherein one or more of the alkyl group's hydrogen atoms has been replaced with an —OH group. In one embodiment, a C₁-C₆alkyl-OH group has from 1 to 6 carbon atoms. Non-limiting examples of hydroxyalkyl groups include —CH₂OH, —CH₂CH₂OH, —CH₂CH₂CH₂OH and —CH₂CH(OH)CH₃.

The term “—O-alkyl” as used herein, refers to an alkoxy group, wherein an alkyl group is as defined above. In one embodiment, a —O—C₁-C₆alkyl is an alkoxy group having from 1 to 6 carbon atoms. Non-limiting examples of alkoxy groups include methoxy, ethoxy, n-propoxy, isopropoxy, n-butoxy and t-butoxy. An alkoxy group is bonded via its oxygen atom.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. In one embodiment, an aromatic or non-aromatic ring system can be substituted with a substituent group wherein the substituent group replaces an available hydrogen on the ring system. Ring system substituents may be the same or different, each being independently selected from the group consisting of alkyl, alkenyl, alkynyl, aryl, heteroaryl, -alkyl-aryl, -aryl-alkyl, -alkyl-heteroaryl, -alkenyl-heteroaryl, -alkynyl-heteroaryl, OH, alkyl-OH, haloalkyl, —O-alkyl, —O-haloalkyl, -alkyl-O-alkyl, —O-aryl, aryl-O-alkyl, acyl, aroyl, halogen, nitro, —CN, —COOH, —C(O)O-alkyl, —C(O)O-aryl, —C(O)O-alkenyl-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkyl-aryl, —S-alkyl-heteroaryl, cycloalkyl, heterocycloalkyl, —O—C(O)-alkyl, —O—C(O)-aryl, —O—C(O)-cycloalkyl, —C(═NH)—NH₂, —C(═NH)—NH(alkyl), Y₁Y₂N—, Y₁Y₂N-alkyl-, Y₁Y₂NC(O)— and —S(O)₂NY₁Y₂, wherein Y₁ and Y₂ can be the same or different and are independently selected from the group consisting of hydrogen, alkyl, aryl, cycloalkyl, and -alkyl-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

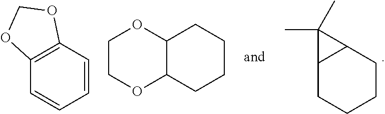

By “stable compound” or “stable structure” is meant a compound that is sufficiently robust to survive isolation to a useful degree of purity from a reaction mixture, and formulation into an efficacious therapeutic agent.

The term “purified”, “in purified form” or “in isolated and purified form” for a compound refers to the physical state of the compound after being isolated from a synthetic process (e.g. from a reaction mixture), or natural source or combination thereof. Thus, the term “purified”, “in purified form” or “in isolated and purified form” for a compound refers to the physical state of the compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any variable (e.g., aryl, heterocycle, R², etc.) occurs more than one time in any constituent or in any of the formulas described herein, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a Bicyclic Heterocycle Derivative or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound described herein contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as O-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a compound described herein contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each a aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound described herein incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N- or di-N,N—(C₁-C₆)alkylaminoalkyl, —C(Y⁴)Y⁵ wherein Y⁴ is H or methyl and Y⁵ is mono-N- or di-N,N—(C₁-C₆)alkylamino morpholine, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The compounds described herein can form salts which are also within the scope of this invention. The term “salt(s)”, as employed herein, denotes acidic salts formed with inorganic and/or organic acids, as well as basic salts formed with inorganic and/or organic bases. In addition, when a compound contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the compounds described herein may be formed by reacting a compound of formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, furnarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, choline, t-butyl amine, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and steatyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (for example, methyl, ethyl, n-propyl, isopropyl, t-butyl, sec-butyl or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the Bicyclic Heterocycle Derivatives may be atropisomers (e.g., substituted biaryls) and are considered as part of this invention. Enantiomers can also be separated by use of chiral HPLC column.

It is also possible that the compounds described herein may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound described herein incorporates a double bond or a fused ring, both the cis- and trans-forms, as well as mixtures, are embraced within the scope of the invention. Also, for example, all keto-enol and imine-enamine forms of the compounds are included in the invention).

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

In the compounds of the formulas described herein, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of the formulas described herein. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic formula can be prepared without undue experimentation by conventional techniques well known to those skilled in the art or by processes analogous to those described in the Examples herein using appropriate isotopically-enriched reagents and/or intermediates.

Polymorphic Rums of the compounds described herein, and of the salts, solvates, hydrates, esters and prodrugs of the compounds described herein, are intended to be included in the present invention.

### Methods of Treatment

Also encompassed by the present invention are methods of treating GPR119-related diseases. The compounds described herein are effective in preventing or treating various GPR119-related diseases, such as metabolic diseases such as type I diabetes, type II diabetes, obesity, inadequate glucose tolerance, insulin resistance, hyperglycemia, hyperlipidemia, hypertriglyceridemia, hypercholesterolemia, dyslipidemia or syndrome X. The compounds described herein are especially useful as a preventive or a remedy for type I diabetes or type II diabetes.

One aspect of the invention described herein provides a method for the treatment and control of obesity or metabolic syndrome, which comprises administering to a patient in need of such treatment a therapeutically effective amount of a compound having the formulas described herein or a pharmaceutically acceptable salt thereof. For example, the compounds described herein are useful for treating or preventing obesity by administering to a subject in need thereof a composition comprising a compound of the formulas described herein.

Methods of treating or preventing obesity and conditions associated with obesity refer to the administration of the pharmaceutical formulations described herein to reduce or maintain the body weight of an obese subject or to reduce or maintain the body weight of an individual at risk of becoming obese. One outcome of treatment may be reducing the body weight of an obese subject relative to that subject's body weight immediately before the administration of the compounds or combinations of the present invention. Another outcome of treatment may be preventing body weight, regain of body weight previously lost as a result of diet, exercise, or pharmacotherapy and preventing weight gain from cessation of smoking. Another outcome of treatment may be decreasing the occurrence of and/or the severity of obesity-related diseases. Yet another outcome of treatment may be decreasing the risk of developing diabetes in an overweight or obese subject. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

Prevention of obesity and obesity-related disorders refers to the administration of the pharmaceutical formulations described herein to reduce or maintain the body weight of a subject at risk of obesity. One outcome of prevention may be reducing the body weight of a subject at risk of obesity relative to that subject's body weight immediately before the administration of the compounds or combinations of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, type 2 diabetes, polycystic ovary disease, cardiovascular diseases, osteoarthritis, dermatological disorders, hypertension, insulin resistance, hypercholesterolemia, hypertriglyceridemia, and cholelithiasis.

Another aspect of the invention that is of interest relates to a method of treating hyperglycemia, diabetes or insulin resistance in a mammalian patient in need of such treatment which comprises administering to said patient a compound in accordance with the formulas described herein or a pharmaceutically acceptable salt thereof in an amount that is effective to treat hyperglycemia, diabetes or insulin resistance.

More particularly, another aspect of the invention that is of interest relates to a method of treating type 2 diabetes in a mammalian patient in need of such treatment comprising administering to the patient a compound in accordance with the formulas described herein or a pharmaceutically acceptable salt thereof in an amount that is effective to treat type 2 diabetes.

Yet another aspect of the invention that is of interest relates to a method of treating non-insulin dependent diabetes mellitus in a mammalian patient in need of such treatment comprising administering to the patient a compound in accordance with the formulas described herein or a pharmaceutically acceptable salt thereof in an amount that is effective to treat non-insulin dependent diabetes mellitus.

The present invention is also directed to the use of a compound of structural formulas described herein in the manufacture of a medicament for use in treating various GPR119-related diseases, such as metabolic diseases such as metabolic diseases such as type I diabetes, type II diabetes, obesity, inadequate glucose tolerance, insulin resistance, hyperglycemia, hyperlipidemia, hypertriglyceridemia, hypercholesterolemia, dyslipidemia or syndrome X. The compounds described herein are especially useful as a preventive or a remedy for type I diabetes and type II diabetes.

For example, the present invention is directed to the use of a compound of the structural formulas described herein in the manufacture of a medicament for use in treating type I diabetes, or type II diabetes.

Additionally, the present invention is directed to the use of a compound of the structural formulas described herein in the manufacture of a medicament for use in treating obesity.

### Compositions

Compounds of the invention may be administered orally or parenterally. As formulated into a dosage form suitable for the administration route, the compound of the invention can be used as a pharmaceutical composition for the prevention, treatment, or remedy of the above diseases.

In clinical use of the compound of the invention, usually, the compound is formulated into various preparations together with pharmaceutically acceptable additives according to the dosage form, and may then be administered. By “pharmaceutically acceptable” it is meant the additive, carrier, diluent or excipient must be compatible with the other ingredients of the formulation and not deleterious to the recipient thereof. As such additives, various additives ordinarily used in the field of pharmaceutical preparations are usable. Specific examples thereof include gelatin, lactose, sucrose, titanium oxide, starch, crystalline cellulose, hydroxypropyl methylcellulose, carboxymethylcellulose, corn starch, microcrystalline wax, white petrolatum, magnesium metasilicate aluminate, anhydrous calcium phosphate, citric acid, trisodium citrate, hydroxypropylcellulose, sorbitol, sorbitan fatty acid ester, polysorbate, sucrose fatty acid ester, polyoxyethylene, hardened castor oil, polyvinylpyrrolidone, magnesium stearate, light silicic acid anhydride, talc, vegetable oil, benzyl alcohol, gum arabic, propylene glycol, polyalkylene glycol, cyclodextrin, hydroxypropyl cyclodextrin, and the like.

Preparations to be formed with those additives include, for example, solid preparations such as tablets, capsules, granules, powders, suppositories; and liquid preparations such as syrups, elixirs, injections. These may be formulated according to conventional methods known in the field of pharmaceutical preparations. The liquid preparations may also be in such a form that may be dissolved or suspended in water or in any other suitable medium in their use. Especially for injections, if desired, the preparations may be dissolved or suspended in physiological saline or glucose liquid, and a buffer or a preservative may be optionally added thereto.

The pharmaceutical compositions may contain the compound of the invention in an amount of from 1 to 99.9% by weight, preferably from 1 to 60% by weight of the composition. The compositions may further contain any other therapeutically-effective compounds.

In cases where the compounds of the invention are used for prevention or treatment for the above-mentioned diseases, the dose and the dosing frequency may be varied, depending on the sex, the age, the body weight and the disease condition of the patient and on the type and the range of the intended remedial effect. In general, when orally administered, the dose may be from 0.001 to 50 mg/kg of body weight/day, and it may be administered at a time or in several times. The dose is preferably from about 0.01 to about 25 mg/kg/day, more preferably from about 0.05 to about 10 mg/kg/day. For oral administration, the compositions are preferably provided in the form of tablets or capsules containing from 0.01 mg to 1,000 mg, preferably 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of a compound described herein. This dosage regimen may be adjusted to provide the optimal therapeutic response.

### Combinations

The compounds of the present invention are further useful in methods for the prevention or treatment of the aforementioned diseases, disorders and conditions in combination with other therapeutic agents.

The compounds of the present invention may be used in combination with one or more other drugs in the treatment, prevention, suppression or amelioration of diseases or conditions for which compounds of the formulas described herein or the other drugs may have utility, where the combination of the drugs together are safer or more effective than either drug alone. Such other drug(s) may be administered, by a route and in an amount commonly used therefore, contemporaneously or sequentially with a compound of the formulas described herein. When a compound of the formulas described herein is used contemporaneously with one or more other drugs, a pharmaceutical composition in unit dosage form containing such other drugs and the compound of the formulas described herein is preferred. However, the combination therapy may also include therapies in which the compound of the formulas described herein and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of the formulas described herein.

Examples of other active ingredients that may be administered in combination with a compound of the formulas described herein, and either administered separately or in the same pharmaceutical composition, include, but are not limited to:

(1) dipeptidyl peptidase-IV (DPP-4) inhibitors;

(2) insulin sensitizers, including (i) PPARγ agonists, such as the glitazones (e.g. pioglitazone, rosiglitazone, netoglitazone, rivoglitazone, and balaglitazone) and other PPAR ligands, including (1) PPARα/γ dual agonists, such as muraglitazar, aleglitazar, sodelglitazar, and naveglitazar, (2) PPARα agonists, such as fenofibric acid derivatives (gemfibrozil, clofibrate, ciprofibrate, fenofibrate and bezafibrate), (3) selective PPARγ modulators (SPPARγM's), such as those disclosed in WO 02/060388, WO 02/08188, WO 2004/019869, WO 2004/020409, WO 2004/020408, and WO 2004/066963, and (4) PPARγ, partial agonists; (ii) biguanides, such as metformin and its pharmaceutically acceptable salts, in particular, metformin hydrochloride, and extended-release formulations thereof, such as Glumetza®, Fortamet®, and GlucophageXR®; (iii) protein tyrosine phosphatase-1B (PTP-1B) inhibitors;

(3) insulin or insulin analogs, such as insulin lispro, insulin detemir, insulin glargine, insulin glulisine, and inhalable formulations of each thereof;

(4) leptin and leptin derivatives and agonists;

(5) amylin and amylin analogs, such as pramlintide;

(6) sulfonylurea and non-sulfonylurea insulin secretagogues, such as tolbutamide, glyburide, glipizide, glimepiride, mitiglinide, and meglitinides, such as nateglinide and repaglinide;

(7) α-glucosidase inhibitors (such as acarbose, voglibose and miglitol);

(8) glucagon receptor antagonists, such as those disclosed in WO 98/04528, WO 99/01423, WO 00/39088, and WO 00/69810;

(9) incretin mimetics, such as GLP-1, GLP-1 analogs, derivatives, and mimetics; and GLP-1 receptor agonists, such as exenatide, liraglutide, taspoglutide, AVE0010, CJC-1131, and BIM-51077, including intranasal, transdermal, and once-weekly formulations thereof;

(10) LDL cholesterol lowering agents such as (1) HMG-CoA reductase inhibitors (lovastatin, simvastatin, pravastatin, cerivastatin, fluvastatin, atorvastatin, pitavastatin, and rosuvastatin), (ii) bile acid sequestering agents (such as cholestyramine, colestimide, colesevelam hydrochloride, colestipol, and dialkylaminoalkyl derivatives of a cross-linked dextran, (iii) inhibitors of cholesterol absorption, such as ezetimibe, and (iv) acyl CoA:cholesterol acyltransferase inhibitors, such as avasimibe;

(11) HDL-raising drugs, such as niacin or a salt thereof and extended-release versions thereof; MK-524A, which is a combination of niacin extended-release and the DP-1 antagonist MK-524; and nicotinic acid receptor agonists;

(12) antiobesity compounds;

(13) agents intended for use in inflammatory conditions, such as aspirin, non-steroidal anti-inflammatory drugs (NSAIDs), glucocorticoids, and selective cyclooxygenase-2 (COX-2) inhibitors;

(14) antihypertensive agents, such as ACE inhibitors (such as enalapril, lisinopril, ramipril, captopril, quinapril, and trandolapril), A-II receptor blockers (such as losartan, candesartan, irbesartan, olmesartan medoxomil, valsartan, tehnisartan, and eprosartan), renin inhibitors (such as aliskiren), beta blockers and calcium channel blockers;

(15) glucokinase activators (GKAs), such as LY2599506;

(16) inhibitors of 11β-hydroxysteroid dehydrogenase type 1, such as those disclosed in U.S. Pat. No. 6,730,690; WO 03/104207; and WO 04/058741;

(17) inhibitors of cholesteryl ester transfer protein (CETP), such as torcetrapib and MK-0859;

(18) inhibitors of fructose 1,6-bisphosphatase, such as those disclosed in U.S. Pat. Nos. 6,054,587; 6,110,903; 6,284,748; 6,399,782; and 6,489,476;

(19) inhibitors of acetyl CoA carboxylase-1 or 2 (ACC1 or ACC2);

(20) AMP-activated Protein Kinase (AMPK) activators;

(21) agonists of the G-protein-coupled receptors: GPR-109, GPR-119, and GPR-40;

(22) SSTR3 antagonists, such as those disclosed in WO 2009/011836;

(23) neuromedin U receptor agonists, such as those disclosed in WO2009/042053, including, but not limited to, neuromedin S (NMS);

(24) inhibitors of stearoyl-coenzyme A delta-9 desaturase (SCD);

(25) GPR-105 antagonists, such as those disclosed in WO 2009/000087;

(26) inhibitors of glucose uptake, such as sodium-glucose transporter (SGLT) inhibitors and its various isoforms, such as SGLT-1; SGLT-2, such as dapagliflozin and remogliflozin; and SGLT-3;

(27) inhibitors of acyl coenzyme A:diacylglycerol acyltransferase 1 and 2 (DGAT-1 and DGAT-2);

(28) inhibitors of fatty acid synthase;

(29) inhibitors of acyl coenzyme A:monoacylglycerol acyltransferase 1 and 2 (MGAT-1 and MGAT-2);

(30) agonists of the TGR5 receptor (also known as GPBAR1, BG37, GPCR19, GPR131, and M-BAR); and

(31) bromocriptine mesylate and rapid-release formulations thereof.

Dipeptidyl peptidase-IV (DPP-4) inhibitors that can be used in combination with compounds of the formulas described herein include, but are not limited to, sitagliptin (disclosed in U.S. Pat. No. 6,699,871), vildagliptin, saxagliptin, alogliptin, denagliptin, carmegliptin, dutogliptin, melogliptin, linagliptin, and pharmaceutically acceptable salts thereof, and fixed-dose combinations of these compounds with metformin hydrochloride, pioglitazone, rosiglitazone, simvastatin, atorvastatin, or a sulfonylurea.

Other dipeptidyl peptidase-IV (DPP-4) inhibitors that can be used in combination with compounds of the formulas described herein include, but are not limited to:
(2R,3S,5R)-5-(1-methyl-4,6-dihydropyrrolo[3,4-c]pyrazol-5(1H)-yl)-2-(2,4,5-trifluorophenyl)tetrahydro-2H-pyran-3-amine;
(2R,3S,5R)-2-(2,5-difluorophenyl)tetrahydro)-5-(4,6-dihydropyrrolo[3,4-c]pyrazol-5(1H)-yl)tetrahydro-2H-pyran-3-amine;
(3R)-4-[(3R)-3-amino-4-(2,4,5-trifluorophenyl)butanoyl]-hexahydro-3-methyl-2H-1,4-diazepin-2-one;
4-[(3R)-3-amino-4-(2,5-difluorophenyl)butanoyl]hexahydro-1-methyl-2H-1,4-diazepin-2-one hydrochloride; and
(3R)-4-[(3R)-3-amino-4-(2,4,5-trifluorophenyl)butanoyl]-hexahydro-3-(2,2,2-trifluoroethyl)-2H-1,4-diazepin-2-one; and
   pharmaceutically acceptable salts thereof.

Antiobesity compounds that can be combined with compounds of the formulas described herein include topiramate; zonisamide; naltrexone; phentermine; bupropion; the combination of bupropion and naltrexone; the combination of bupropion and zonisamide; the combination of topiramate and phentermine; fenfluramine; dexfenfluramine; sibutramine; lipase inhibitors, such as orlistat and cetilistat; melanocortin receptor agonists, in particular, melanocortin-4 receptor agonists; CCK-1 agonists; melanin-concentrating hormone (MCH) receptor antagonists; neuropeptide Y₁ or Y₅ antagonists (such as MK-0557); CB1 receptor inverse agonists and antagonists (such as rimonabant and taranabant); β₃ adrenergic receptor agonists; ghrelin antagonists; bombesin receptor agonists (such as bombesin receptor subtype-3 agonists); and 5-hydroxytryptamine-2c (5-HT2c) agonists, such as lorcaserin. For a review of anti-obesity compounds that can be combined with compounds of the present invention, see S. Chaki et al., “Recent advances in feeding suppressing agents: potential therapeutic strategy for the treatment of obesity,” Expert Opin. Ther. Patents, 11: 1677-1692 (2001); D. Spanswick and K. Lee, “Emerging antiobesity drugs,” Expert Opin. Emerging Drugs, 8: 217-237 (2003); J. A. Fernandez-Lopez, et al., “Pharmacological Approaches for the Treatment of Obesity,” Drugs, 62: 915-944 (2002); and K. M. Gadde, et al., “Combination pharmaceutical therapies for obesity,” Exp. Opin. Pharmacother., 10: 921-925 (2009).

Glucagon receptor antagonists that can be used in combination with the compounds of the formulas described herein include, but are not limited to:
N-[4-((1S)-1-{3-(3,5-dichlorophenyl)-5-[6-(trifluoromethoxy)-2-naphthyl]-1H-pyrazol-1-yl}ethyl)benzoyl]-β-alanine;
N-[4-((1R)-1-{3-(3,5-dichlorophenyl)-5-[6-(trifluoromethoxy)-2-naphthyl]-1H-pyrazol-1-yl}ethyl)benzoyl]-β-alanine;
N-(4-{1-[3-(2,5-dichlorophenyl)-5-(6-methoxy-2-naphthyl)-1H-pyrazol-1-yl]ethyl}benzoyl)-β-alanine;
N-(4-{(1S)-1-[3-(3,5-dichlorophenyl)-5-(6-methoxy-2-naphthyl)-1H-pyrazol-1-yl]ethyl}benzoyl)-β-alanine;
N-(4-{(1S)-1-[(R)-(4-chlorophenyl)(7-fluoro-5-methyl-1H-indol-3-yl)methyl]butyl}benzoyl)-β-alanine; and
N-(4-{(1S)-1-[(4-chlorophenyl)(6-chloro-8-methylquinolin-4-yl)methyl]butyl}benzoyl)-β-alanine; and
   pharmaceutically acceptable salts thereof.

Inhibitors of stearoyl-coenzyme A delta-9 desaturase (SCD) that can be used in combination with the compounds of the formulas described herein include, but are not limited to:
[5-(5-{4-[2-(trifluoromethyl)phenoxy]piperidin-1-yl}-1,3,4-thiadiazol-2-yl)-2H-tetrazol-2-yl]acetic acid;
(2′-{4-[2-(trifluoromethyl)phenoxy]piperidin-1-yl}-2,5′-bi-1,3-thiazol-4-yl)acetic acid;
(5-{3-[4-(2-bromo-5-fluorophenoxy)piperidin-1-yl]isoxazol-5-yl}-2H-tetrazol-2-yl)acetic acid;
(3-{3-[4-(2-bromo-5-fluorophenoxy)piperidin-1-yl]-1,2,4-oxadiazol-5-yl}-1H-pyrrol-1-yl)acetic acid;
(5-{5-[4-(2-bromo-5-fluorophenoxy)piperidin-1-yl]pyrazin-2-yl}-2H-tetrazol-2-yl)acetic acid; and
(5-{2-[4-(5-bromo-2-chlorophenoxy)piperidin-1-yl]pyrimidin-5-yl}-2H-tetrazol-2-yl)acetic acid;
   and pharmaceutically acceptable salts thereof.

Glucokinase activators that can be used in combination with the compounds of the formulas described herein include, but are not limited to:
3-(6-ethanesulfonylpyridin-3-yloxy)-5-(2-hydroxy-1-methyl-ethoxy)-N-(1-methyl-1H-pyrazol-3-yl)benzamide;
5-(2-hydroxy-1-methyl-ethoxy)-3-(6-methanesulfonylpyridin-3-yloxy)-N-(1-methyl-1H-pyrazol-3-yl)benzamide;
5-(1-hydroxymethyl-propoxy)-3-(6-methanesulfonylpyridin-3-yloxy)-N-(1-methyl-1H-pyrazol-3-yl)benzamide;
3-(6-methanesulfonylpyridin-3-yloxy)-5-(1-methoxymethyl-propoxy)-N-(1-methyl-1H-pyrazol-3-yl)benzamide;
5-isopropoxy-3-(6-methanesulfonylpyridin-3-yloxy)-N-(1-methyl-1H-pyrazol-3-yl)benzamide;
5-(2-fluoro-1-fluoromethyl-ethoxy)-3-(6-methanesulfonylpyridin-3-yloxy)-N-(1-methyl-1H-pyrazol-3-yl)benzamide;
3-({4-[2-(dimethylamino)ethoxy]phenyl}thio)-N-(3-methyl-1,2,4-thiadiazol-5-yl)-6-[(4-methyl-4H-1,2,4-triazol-3-yl)thio]pyridine-2-carboxamide;
3-({4-[(1-methylazetidin-3-yl)oxy]phenyl}thio)-N-(3-methyl-1,2,4-thiadiazol-5-yl)-6-[(4-methyl-4H-1,2,4-triazol-3-yl)thio]pyridine-2-carboxamide;
N-(3-methyl-1,2,4-thiadiazol-5-yl)-6-[(4-methyl-4H-1,2,4-triazol-3-yl)thio]-3-{[4-(2-pyrrolidin-1-ylethoxy)phenyl]thio}pyridine-2-carboxamide; and
3-[(4-{2-[(2R)-2-methylpyrrolidin-1-yl]ethoxy}phenyl)thio-N-(3-methyl-1,2,4-thiadiazol-5-yl)-6-[(4-methyl-4H-1,2,4-triazol-3-yl)thio]pyridine-2-carboxamide; and pharmaceutically acceptable salts thereof.

Agonists of the GPR-119 receptor that can be used in combination with the compounds of the formulas described herein include, but are not limited to:
rac-cis 5-chloro-2-{4-[2-(2-{[5-(methylsulfonyl)pyridin-2-yl]oxy}ethyl)cyclopropyl]piperidin-1-yl}pyrimidine;
5-chloro-2-{4-[(1R,2S)-2-(2-{[5-(methylsulfonyppyridin-2-yl]oxy}ethypcyclopropyl]piperidin-1-yl}pyrimidine;
rac cis-5-chloro-2-[4-(2-{2-[4-(methylsulfonyl)phenoxy]ethyl}cyclopropyl)piperidin-1-yl]pyrimidine;
5-chloro-2-[4-((1S,2R)-2-{2-[4-(methylsulfonyl)phenoxy]ethyl}cyclopropyl)piperidin-1-yl]pyrimidine;
5-chloro-2-[4-((1R,2S)-2-{2-[4-(methylsulfonyl)phenoxy]ethyl}cyclopropyl)piperidin-1-yl]pyrimidine;
rac cis-5-chloro-2-[4-(2-{2-[3-(methyl sulfonyl)phenoxy]ethyl}cyclopropyl)piperidin-1-yl]pyrimidine; and
rac cis-5-chloro-2-[4-(2-{2-[3-(5-methyl-1,3,4-oxadiazol-2-yl)phenoxy]ethyl}cyclopropyl)piperidin-1-yl]pyrimidine; and pharmaceutically acceptable salts thereof.

Selective PPARγ modulators (SPPARγM's) that can be used in combination with the compounds of the formulas described herein include, but are not limited to:
(2S)-2-({6-chloro-3-[6-(4-chlorophenoxy)-2-propylpyridin-3-yl]-1,2-benzisoxazol-5-yl}oxy)propanoic acid;
(2S)-2-({6-chloro-3-[6-(4-fluorophenoxy)-2-propylpyridin-3-yl]-1,2-benzisoxazol-5-yl}oxy)propanoic acid;
(2S)-2-{[6-chloro-3-(6-phenoxy-2-propylpyridin-3-yl)-1,2-benzisoxazol-5-yl]oxy}propanoic acid;
(2R)-2-({6-chloro-3-[6-(4-chlorophenoxy)-2-propylpyridin-3-yl]-1,2-benzisoxazol-5-yl}oxy)propanoic acid;
(2R)-2-{3-[3-(4-methoxy)benzoyl-2-methyl-6-(trifluoromethoxy)-1H-indol-1-yl]phenoxy}butanoic acid;
(2S)-2-{3-[3-(4-methoxy)benzoyl-2-methyl-6-(trifluoromethoxy)-1H-indol-1-yl]phenoxy}butanoic acid;
2-{3-[3-(4-methoxy)benzoyl-2-methyl-6-(trifluoromethoxy)-1H-indol-1-yl]phenoxy}-2-methylpropanoic acid; and
(2R)-2-{3-[3-(4-chloro)benzoyl-2-methyl-6-(trifluorornethoxy)-1H-indol-1-yl]phenoxy}propanoic acid; and pharmaceutically acceptable salts thereof.

Inhibitors of 11β-hydroxysteroid dehydrogenase type 1 that can be used in combination with the compounds of the formulas described herein include, but are not limited to:
3-[1-(4-chlorophenyl)-trans-3-fluorocyclobutyl]-4,5-dicyclopropyl-r-4H-1,2,4-triazole; 3-[1-(4-chlorophenyl)-trans-3-fluorocyclobutyl]-4-cyclopropyl-5-(1-methylcyclopropyl)-r-4H-1,2,4-triazole;
3-[1-(4-chlorophenyl)-trans-3-fluorocyclobutyl]-4-methyl-5-[2-(trifluoromethoxy)phenyl]-r-4H-1,2,4-triazole;
3-[1-(4-chlorophenyl)cyclobutyl]-4-methyl-5-[2-(trifluoromethyl)phenyl]-4H-1,2,4-triazole;
3-{4-[3-(ethylsulfonyl)propyl]bicyclo[2.2.2]oct-1-yl}-4-methyl-5-[2-(trifluoromethyl)phenyl]-4H-1,2,4-triazole;
4-methyl-3-{4-[4-(methylsulfonyl)phenyl]bicyclo[2.2.2]oct-1-yl}-5-[2-(trifluoromethyl)phenyl]-4H-1,2,4-triazole;
3-(4-{4-methyl-5-[2-(trifluoromethyl)phenyl]-4H-1,2,4-triazol-3-yl}bicyclo[2.2.2]oct-1-yl)-5-(3,3,3-trifluoropropyl)-1,2,4-oxadiazole;
3-(4-{4-methyl-5-[2-(trifluoromethyl)phenyl]-4H-1,2,4-triazol-3-yl}bicyclo[2.2.2]oct-1-yl)-5-(2,2,2-trifluoroethyl)-1,2,4-oxadiazole;
5-(3,3-difluorocyclobutyl)-3-(4-{4-methyl-5-[2-(trifluoromethyl)phenyl]-4H-1,2,4-triazol-3-yl}bicyclo[2.2.2]oct-1-yl)-1,2,4-oxadiazole;
5-(1-fluoro-1-methylethyl)-3-(4-{4-methyl-5-[2-(trifluoromethyl)phenyl]-4H-1,2,4-triazol-3-yl}bicyclo[2.2.2]oct-1-yl)-1,2,4-oxadiazole;
2-(1,1-difluoroethyl)-5-(4-{4-methyl-5-[2-(trifluoromethyl)phenyl]-4H-1,2,4-triazol-3-yl}bicyclo[2.2.2]oct-1-yl)-1,3,4-oxadiazole;
2-(3,3-difluorocyclobutyl)-5-(4-{4-methyl-5-[2-(trifluoromethyl)phenyl]-4H-1,2,4-triazol-3-yl}bicyclo[2.2.2]oct-1-yl)-1,3,4-oxadiazole; and
5-(1,1-difluoroethyl)-3-(4-{4-methyl-5-[2-(trifluoromethyl)phenyl]-4H-1,2,4-triazol-3-yl}bicyclo[2.2.2]oct-1-yl)-1,2,4-oxadiazole; and pharmaceutically acceptable salts thereof.

Somatostatin subtype receptor 3 (SSTR3) antagonists that can be used in combination with the compounds of the formulas described herein include, but are not limited to:

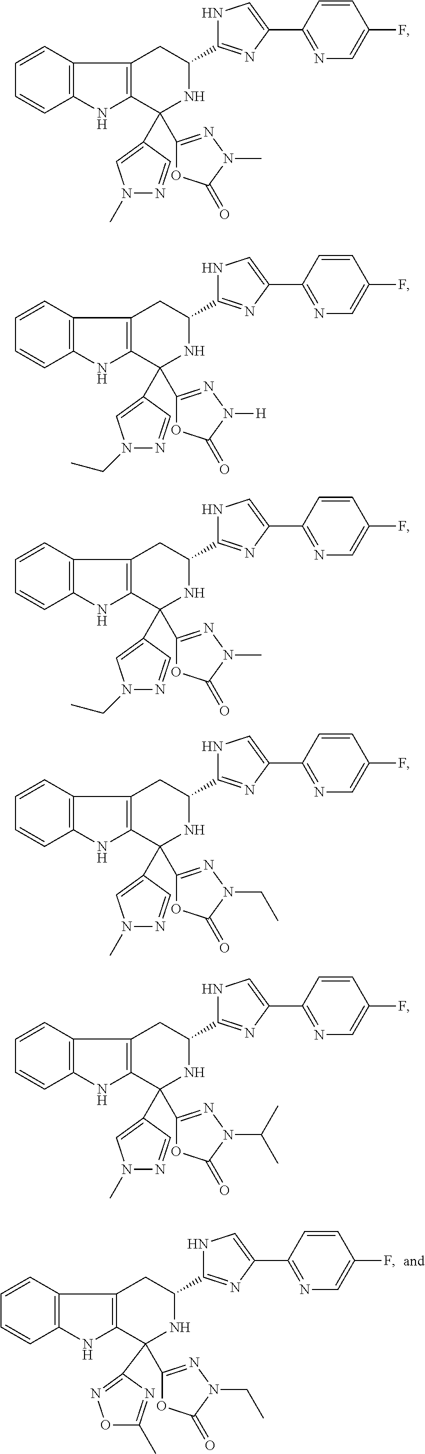

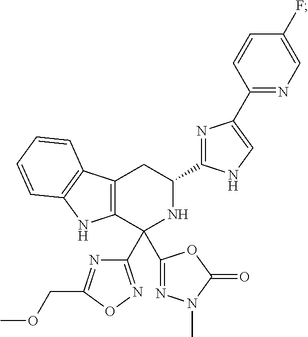 [SMILES: COCc1nc(C2(c3nn(C)c(=O)o3)N[C@@H](c3nc(-c4ccc(F)cn4)c[nH]3)Cc3c2[nH]c2ccccc32)no1]

and pharmaceutically acceptable salts thereof.

AMP-activated Protein Kinase (AMPK) activators that can be used in combination with the compounds of the formulas described herein include, but are not limited to:

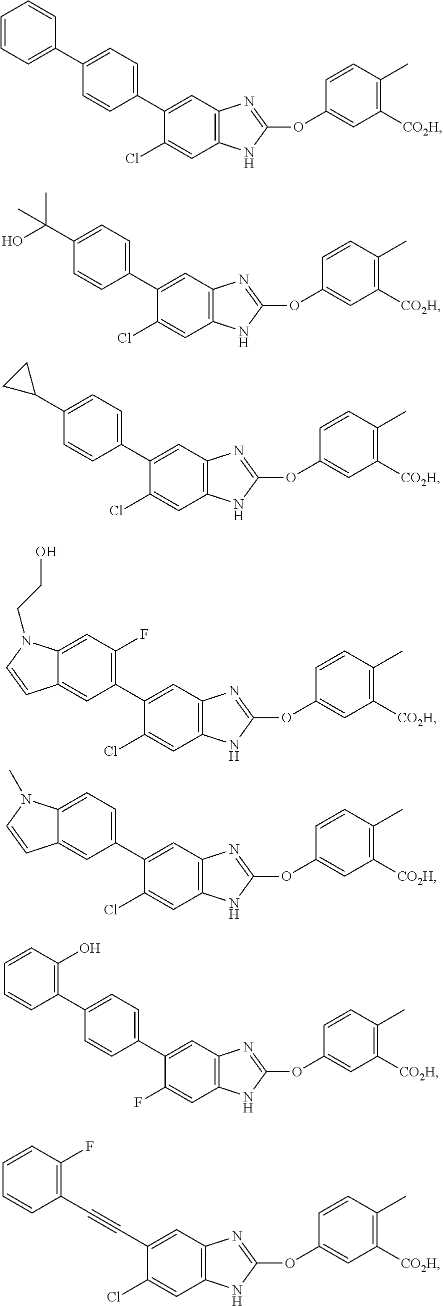

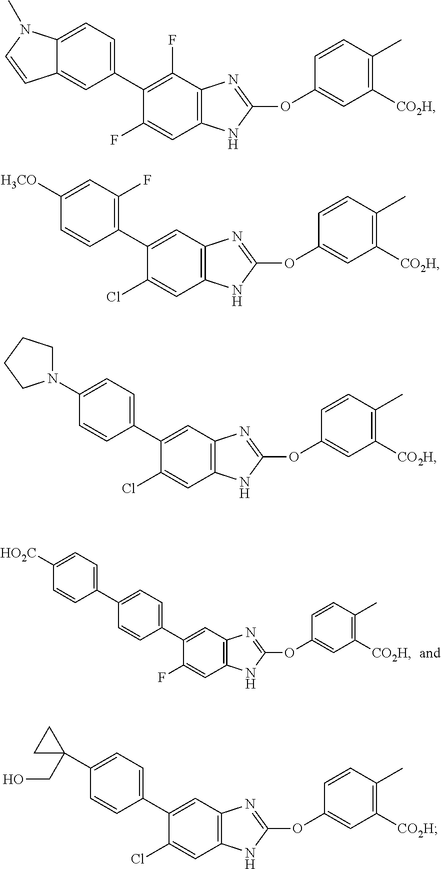 [SMILES: Cc1ccc(Oc2nc3c(F)c(-c4ccc5c(ccn5C)c4)c(F)cc3[nH]2)cc1C(=O)O ; COc1ccc(-c2cc3nc(Oc4ccc(C)c(C(=O)O)c4)[nH]c3cc2Cl)c(F)c1 ; Cc1ccc(Oc2nc3cc(-c4ccc(N5CCCC5)cc4)c(Cl)cc3[nH]2)cc1C(=O)O ; Cc1ccc(Oc2nc3cc(-c4ccc(-c5ccc(C(=O)O)cc5)cc4)c(F)cc3[nH]2)cc1C(=O)O ; "and" ; Cc1ccc(Oc2nc3cc(-c4ccc(C5(CO)CC5)cc4)c(Cl)cc3[nH]2)cc1C(=O)O]

and pharmaceutically acceptable salts thereof.

Inhibitors of acetyl-CoA carboxylase-1 and 2 (ACC-1 and ACC-2) that can be used in combination with the compounds of the formulas described herein include, but are not limited to:
3-{1′-[(1-cyclopropyl-4-methoxy-1H-indol-6-yl)carbonyl]-4-oxospiro[chroman-2,4′-piperidin]-6-yl}benzoic acid;
5-{1′-[(1-cyclopropyl-4-methoxy-H-indol-6-yl)carbonyl]-4-oxospiro[chroman-2,4′-piperidin]-6-yl}nicotinic acid;
1′[(1-cyclopropyl-4-methoxy-1H-indol-6-yl)carbonyl]-6-(1H-tetrazol-5-yl)spiro[chroman-2,4′-piperidin]-4-one;
1-[(1-cyclopropyl-4-ethoxy-3-methyl-1H-indol-6-yl)carbonyl]-6-(1H-tetrazol-5-yl)spiro[chroman-2,4′-piperidin]-4-one; and
5-{1′-[(1-cyclopropyl-4-methoxy-3-methyl-1,1-indol-6-yl)carbonyl]-4-oxo-spiro[chroman-2,4′-piperidin]-6-yl}nicotinic acid; and
   pharmaceutically acceptable salts thereof.

In another aspect of the invention, a pharmaceutical composition is disclosed which comprises one or more of the following agents:

(a) a compound of structural formula I or formula Ia or formula Ib;

(b) one or more compounds selected from the group consisting of:
(1) dipeptidyl peptidase-IV (DPP-4) inhibitors;
(2) insulin sensitizers, including (i) PPARy agonists, such as the glitazones (e.g. pioglitazone, rosiglitazone, netoglitazone, rivoglitazone, and balaglitazone) and other PPAR ligands, including (1) PPARα/γ dual agonists, such as muraglitazar, aleglitazar, sodelglitazar, and naveglitazar, (2) PPARα agonists, such as fenofibric acid derivatives (gemfibrozil, clofibrate, ciprofibrate, fenofibrate and bezafibrate), (3) selective PPARγ modulators (SPPARγM's), and (4) PPARγ partial agonists; (ii) biguanides, such as metformin and its pharmaceutically acceptable salts, in particular, metformin hydrochloride, and extended-release formulations thereof, such as Glumetza®, Fortamet®, and GlucophageXR®; (iii) protein tyrosine phosphatase-1B (PTP-1B) inhibitors;
(3) sulfonylurea and non-sulfonylurea insulin secretagogues, such as tolbutamide, glyburide, glipizide, glimepiride, mitiglinide, and meglitinides, such as nateglinide and repaglinide;
(4) α-glucosidase inhibitors (such as acarbose, voglibose and miglitol);
(5) glucagon receptor antagonists;
(6) LDL cholesterol lowering agents such as (i) HMG-CoA reductase inhibitors (lovastatin, simvastatin, pravastatin, cerivastatin, fluvastatin, atorvastatin, pitavastatin, and rosuvastatin), (ii) bile acid sequestering agents (such as cholestyramine, colestimide, colesevelam hydrochloride, colestipol, and dialkylaminoalkyl derivatives of a cross-linked dextran, (iii) inhibitors of cholesterol absorption, such as ezetimibe, and (iv) acyl CoA:cholesterol acyltransferase inhibitors, such as avasimibe;
(7) HDL-raising drugs, such as niacin or a salt thereof and extended-release versions thereof; MK-524A, which is a combination of niacin extended-release and the DP-1 antagonist MK-524; and nicotinic acid receptor agonists;
(8) antiobesity compounds;
(9) agents intended for use in inflammatory conditions, such as aspirin, non-steroidal anti-inflammatory drugs (NSAIDs), glucocorticoids, and selective cyclooxygenase-2 (COX-2) inhibitors;
(10) antihypertensive agents, such as ACE inhibitors (such as enalapril, lisinopril, ramipril, captopril, quinapril, and tandolapril), A-II receptor blockers (such as losartan, candesartan, irbesartan, olmesartan medoxomil, valsartan, telmisartan, and eprosartan), renin inhibitors (such as aliskiren), beta blockers (such as and calcium channel blockers (such as;
(11) glucokinase activators (GKAs), such as LY2599506;
(12) inhibitors of 11β-hydroxysteroid dehydrogenase type 1;
(13) inhibitors of cholesteryl ester transfer protein (CETP), such as torcetrapib and MK-0859;
(14) inhibitors of fructose 1,6-bisphosphatase;
(15) inhibitors of acetyl CoA carboxylase-1 or 2 (ACC1 or ACC2);
(16) AMP-activated Protein Kinase (AMPK) activators;
(17) agonists of the G-protein-coupled receptors: GPR-109, GPR-119, and GPR-40;
(18) SSTR3 antagonists;
(19) neuromedin U receptor agonists, including, but not limited to, neuromedin S (NMS);
(20) inhibitors of stearoyl-coenzyme A delta-9 desaturase (SCD);
(21) GPR-105 antagonists;
(22) inhibitors of glucose uptake, such as sodium-glucose transporter (SGLT) inhibitors and its various isoforms, such as SGLT-1; SGLT-2, such as dapagliflozin and remogliflozin; and SGLT-3;
(23) inhibitors of acyl coenzyme A:diacylglycerol acyltransferase 1 and 2 (DGAT-1 and DGAT-2);
(24) inhibitors of fatty acid synthase;
(25) inhibitors of acetyl-CoA carboxylase-1 and 2 (ACC-1 and ACC-2);
(26) inhibitors of acyl coenzyme A:monoacylglycerol acyltransferase 1 and 2 (MGAT-1 and MGAT-2);
(27) agonists of the TGRS receptor (also known as GPBAR1, B037, GPCR19, GPR131, and M-BAR); and
(28) bromocriptine mesylate and rapid-release formulations thereof; and

(c) a pharmaceutically acceptable carrier.

When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

### EXAMPLES

These compounds may be prepared by a variety of methods, One method is illustrated in Scheme 1. In this method, a bicyclic pyrimidine or pyridine derivative of type 1 is brominated, typically with N-bromosuccinimide at ambient temperature, to provide bromo-derivative 2. When X in compounds I is NH, it may be desirable to protect the NH as N-Boc at this stage. Compound 2 is next condensed with an alcohol 3, typically under the influence of a base such as sodium t-butoxide at temperature from ambient to 100° C. in a solvent such as dioxane. Ether 4 is then arylated to provide 5, typically with an aryl-boronic acid or aryl-boronate ester. Preferred conditions are Pd-catalysis (such as Pd(dppf)Cl₂) in aqueous dioxane with K₂CO₃ at temperatures from 80 to 120° C.

In the preparation of compounds 5, W′ may represent the group W—³ present in I, or it may represent a protective group such as Boc. In the latter case, the protective group may be removed by known deprotection methods, such as HCl for a Boc group, providing 5 where W′ is H. In this case, the W—R³ group is introduced by methods well known in the art, such as reaction with a heteroaryl halide when W—R³ is a heteroaryl group, or reaction with an alkyl or cycloalkyl chloroformate (or hydroxysuccinimide ester) when W—R³ is an alkoxycarbonyl or cycloalkoxycarbonyl group, When R³ is haloalkyl, reaction with a haloalkyl sulfonate ester may be employed. These procedures provide compounds of structure Ic.

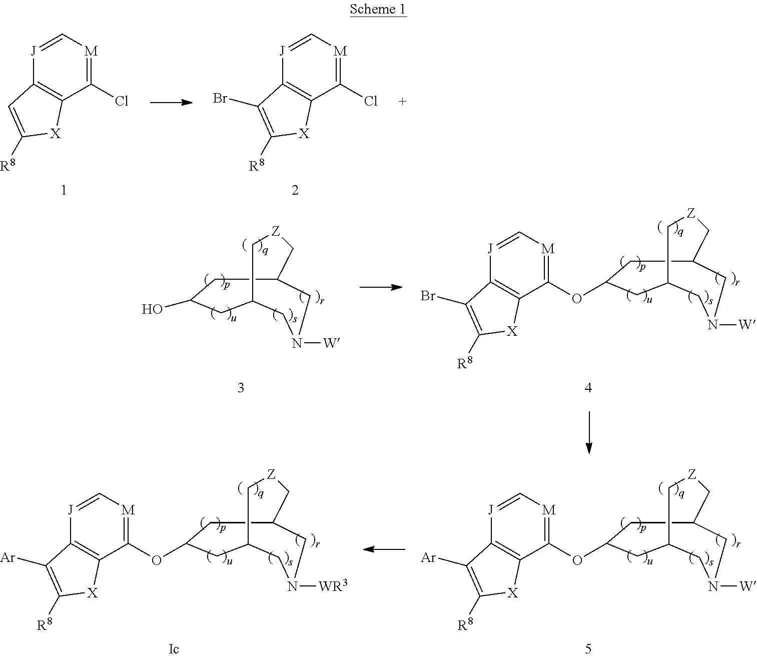

It is also possible to interconvert substituents on the Ar group by well known methods, such as —CN to cycloalkylcarbonyl by reaction with a cycloalkyl Grignard reagent, or halogen to heteroaryl by reaction with an NH heteroaryl and base or reaction with a heteroaryl boronic acid and palladium catalyst.

The compounds of structure I can exist as synlanti or exolendo isomers relative to the saturated bicyclic ring system. All such isomers are contemplated.

The following abbreviations are used below and have the following meanings: AcOH is acetic acid, BINAP is [1,1′-binaphthalene]-2,2′-diyibis(diphenylphosphine), Boe or BOC is —C(O)O-(t-butyl), t-butyl is tertiary butyl, DCM is dichloromethane, DMEM is Dulbecco's modified eagle medium, DME is dimethoxyethane, DMF is N,N-dimethylformamide, DMSO is dimethylsulfoxide, EtMgBr is ethyl magnesium bromide, Et₂O is diethyl ether, EtOAc is ethyl acetate, EtOH is ethanol, Et₃N is triethylamine, LCMS is liquid chromatography mass spectrometry, MeOH is methanol, Na(OAc)₃BH is sodium triacetoxy borohydride, NaOtBu is sodium t-butoxide, NMR is nuclear magnetic resonance, Pd(dppf)Cl₂ is dichloro-((bis-diphenylphosphino)ferrocenyl)palladium(II), Ph is phenyl, PhMe is toluene, PLC is preparative layer chromatography, TFA is trifluoroacetic acid, THF is tetrahydrofuran, TLC is thin-layer chromatography, TMST is trimethylsilyl iodide, and Ti(OPr-i)₄ is titanium tetraisopropoxide.

### Intermediate 1

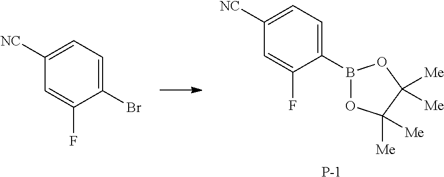

4-Bromo-3-fluorobenzonitrile (1.61 g, 8.1 mmol) was combined with bis(pinacolato)diboron (2.05 g, 8.1 mmol), NaOAc (0.79 g, 9.6 mmol) and Pd(dppf)Cl₂ (0.18 g, 0.24 mmol) in DME (10 mL). The mixture was heated by microwave at 150° C. 1 h, concentrated, and chromatographed on silica (0-10% EtOAc/hexane) to give Intermediate P-1 as a colorless oil.

### Intermediate 2

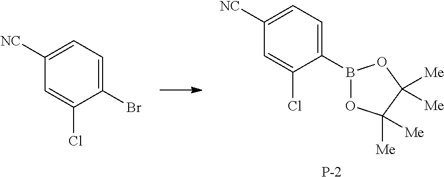

In similar fashion to Intermediate P-1,4-bromo-3-chlorobenzonitrile was converted to Intermediate P-2 as a white solid.

### Example 1

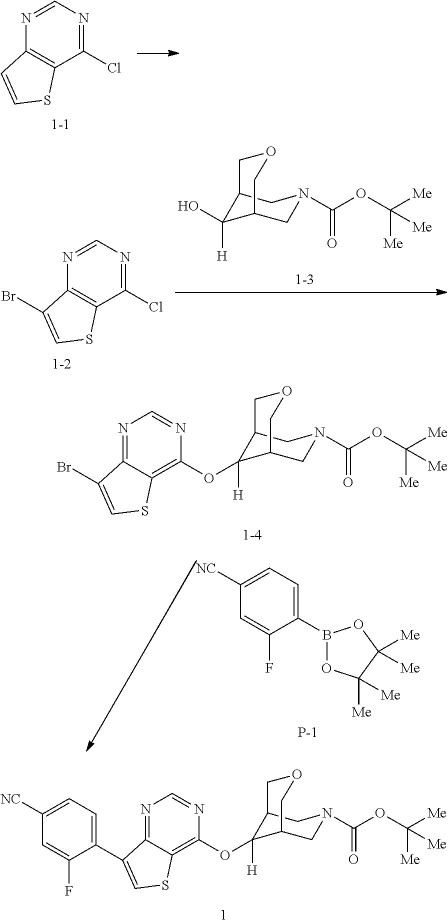

Step 1. Compound 1-1 (0.80 g, 4.7 mmol) was combined with N-bromosuccinimde (1.01 g, 5.7 mmol) and HOAc (0.2 mL) in MeCN (20 mL). The mixture was heated in an 85° C. bath 18 h, concentrated, and purified by PLC to yield Compound 1-2 as a yellow solid.

Step 2. Compound 1-2 (0.52 g, 2.1 mmol) was combined with known Compound 1-3 (0.61 g, 2.5 mmol) and NaO-t-Bu (0.24 g, 2.5 mmol) in dioxane (15 mL) and the mixture heated at 70° C. 2 h, allowed to cool, and partitioned with EtOAc and water. Drying (MgSO₄) and concentration gave Compound 1-4 as a yellow solid.

Step 3. Compound 1-4 (0.30 g, 0.66 mmol) was combined with Intermediate P-1 (0.24 g, 0.98 mmol), K₂CO₃ (0.18 g, 1.3 mmol) and Pd(dppf)Cl₂ (0.048 g, 0.066 mmol) in dioxane (4.0 mL) and water (0.3 mL). The mixture was heated at 100° C. 18 h, concentrated, and purified by PLC to yield Compound 1 as a white solid, LC-MS: m/e 497 (M+1).

### Example 2

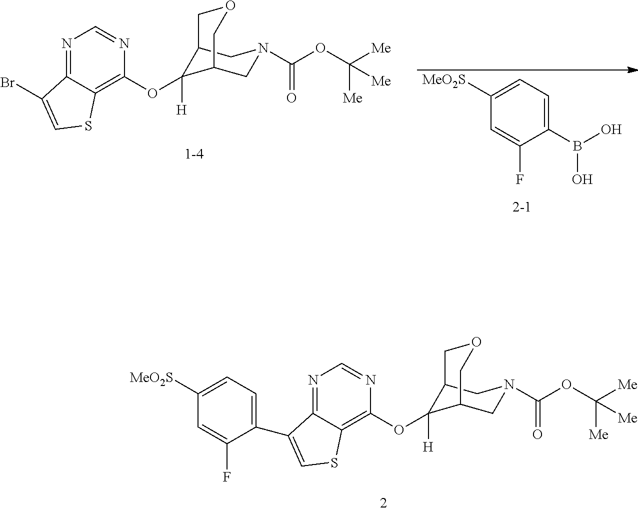 [SMILES: CC(C)(C)OC(=O)N1CC2COCC(C1)C2Oc1ncnc2c(Br)csc12 ; CS(=O)(=O)c1ccc(B(O)O)c(F)c1 ; CC(C)(C)OC(=O)N1CC2COCC(C1)C2Oc1ncnc2c(-c3ccc(S(C)(=O)=O)cc3F)csc12]

Compound 1-4 (0.20 g, 0.44 mmol) was treated with Compound 2-1 (0.143 g, 0.66 mmol), under the conditions of Example 1, Step 3. Purification by PLC (3% MeOH/CH₂Cl₂) yielded

Compound 2 as a white solid, LC-MS: m/e 550 (M+1).

### Example 3

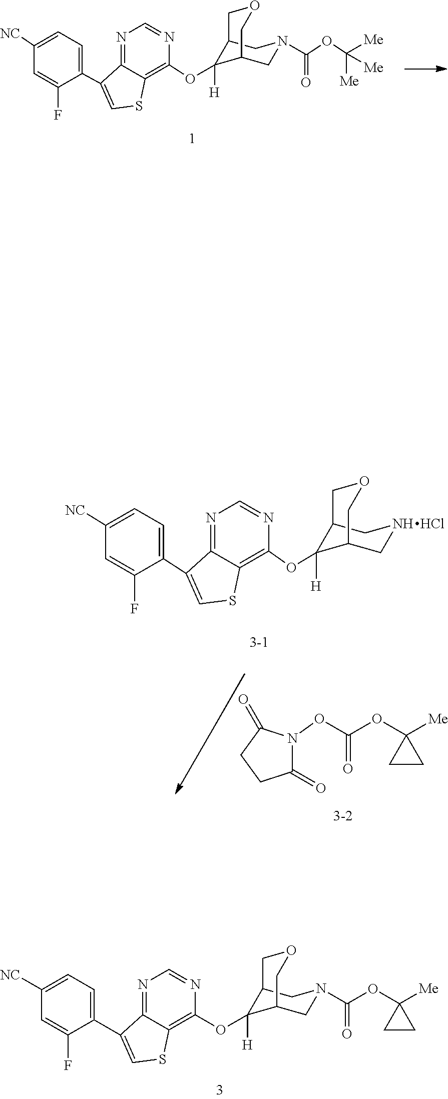 [SMILES: CC(C)(C)OC(=O)N1CC2COCC(C1)C2Oc1ncnc2c(-c3ccc(C#N)cc3F)csc12 ; N#Cc1ccc(-c2csc3c(OC4C5CNCC4COC5)ncnc23)c(F)c1 ; Cl ; CC1(OC(=O)ON2C(=O)CCC2=O)CC1 ; CC1(OC(=O)N2CC3COCC(C2)C3Oc2ncnc3c(-c4ccc(C#N)cc4F)csc23)CC1]

Step 1: To Compound 1 (0.075 g, 0.15 mmol) in CH₂Cl₂ (2.0 mL) was added 4.0M HCl/dioxane (2.0 mL). The solution was stirred 2 h and concentrated to give Compound 3-1.

Step 2: The material from Step 1 in CH₂Cl₂ (3.0 mL) was combined with Et₃N (0.10 mL, 0.72 mmol) and 1-methylcyclopropyl hydroxysuccinimidyl carbonate (3-2, 0.048 g, 0.22 mmol). After 1 h, concentration and purification by PLC yielded Compound 3 as a white solid, LC-MS: m/e 495 (M+1).

### Example 4

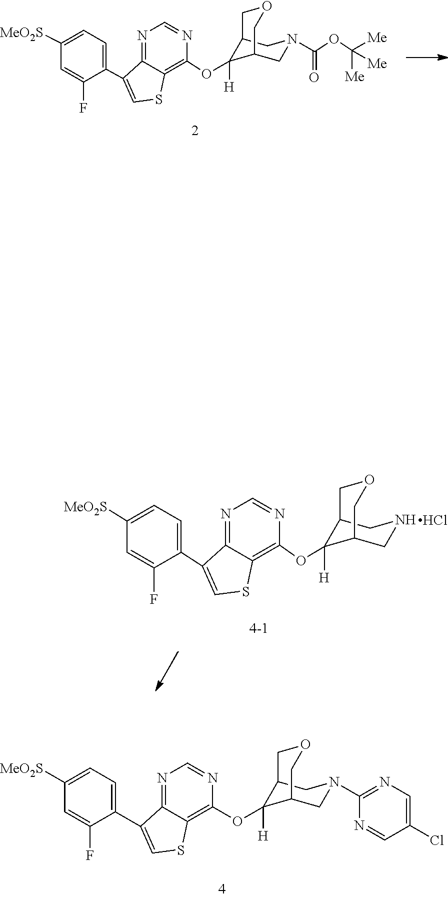 [SMILES: CC(C)(C)OC(=O)N1CC2COCC(C1)C2Oc1ncnc2c(-c3ccc(S(C)(=O)=O)cc3F)csc12 ; CS(=O)(=O)c1ccc(-c2csc3c(OC4C5CNCC4COC5)ncnc23)c(F)c1 ; Cl ; CS(=O)(=O)c1ccc(-c2csc3c(OC4C5COCC4CN(c4ncc(Cl)cn4)C5)ncnc23)c(F)c1]

Step 1: In similar manner to Example 3, Step 1, Compound 2 (0.075 g, 0.15 mmol) was converted to Compound 4-1.

Step 2: To the material from Step 1 in DMSO (3.0 mL) were added K₂CO₃ (0.100 g, 0.72 mmol), then AgF (0.032 g, 0.25 mmol), then 2,5-dichloropyrimidine (0.037 g, 0.25 mmol). The mixture was heated with stirring at 130° C. 1 h, allowed to cool, and concentrated. Purification by PLC (7% acetone/CH₂Cl₂) yielded Compound 4 as a white solid, LC-MS: in/e 562+564 (M+1).

### Example 5

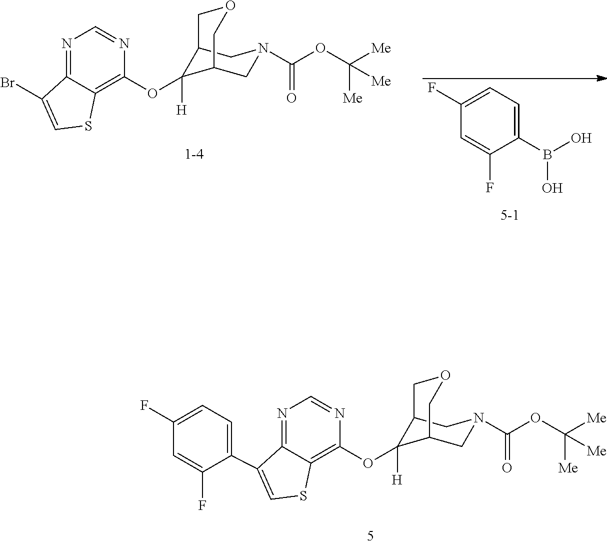 [SMILES: CC(C)(C)OC(=O)N1CC2COCC(C1)C2Oc1ncnc2c(Br)csc12 ; OB(O)c1ccc(F)cc1F ; CC(C)(C)OC(=O)N1CC2COCC(C1)C2Oc1ncnc2c(-c3ccc(F)cc3F)csc12]

In similar fashion to Example 1, Step 3, Compound 1-4 (0.20 g, 0.44 mmol) and Compound 5-1 (0.14 g, 0.88 mmol) were heated by microwave with the other components at 120° C. 1 h. Purification by PLC (3% MeOH/CH₂Cl₂) yielded Compound 5 as a yellow solid, LC-MS: mile 490 (M+1).

### Example 6

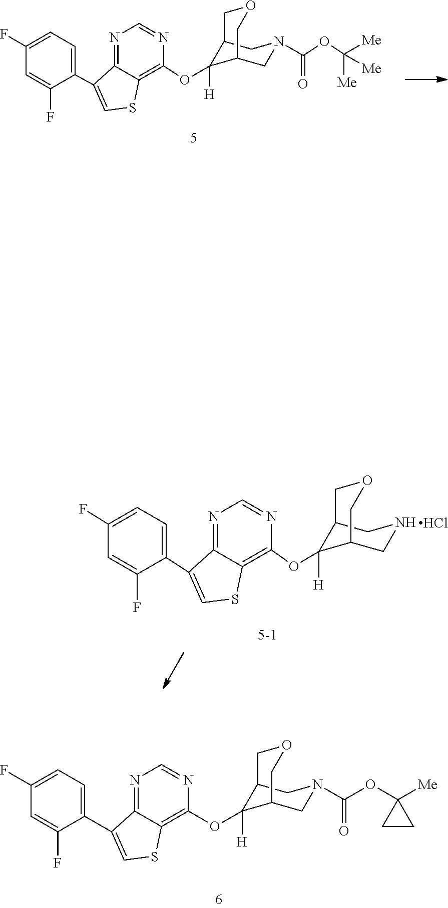 [SMILES: CC(C)(C)OC(=O)N1CC2COCC(C1)C2Oc1ncnc2c(-c3ccc(F)cc3F)csc12 ; Fc1ccc(-c2csc3c(OC4C5CNCC4COC5)ncnc23)c(F)c1 ; Cl ; CC1(OC(=O)N2CC3COCC(C2)C3Oc2ncnc3c(-c4ccc(F)cc4F)csc23)CC1]

In similar fashion to Example 3, Compound 5 was converted into Compound 6, a white solid, LC-MS: m/e 488 (M+1).

### Example 7

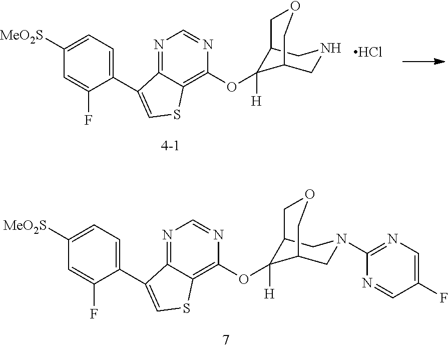 [SMILES: CS(=O)(=O)c1ccc(-c2csc3c(OC4C5CNCC4COC5)ncnc23)c(F)c1 ; Cl ; CS(=O)(=O)c1ccc(-c2csc3c(OC4C5COCC4CN(c4ncc(F)cn4)C5)ncnc23)c(F)c1]

In similar fashion to Example 4, Step 2, Compound 4-1 was treated with 2-chloro-5-fluoropyrimidine (heating at 110° C. 18 h) to provide Compound 7, a white solid, LC-MS: rale 546 (M+1).

### Example 8

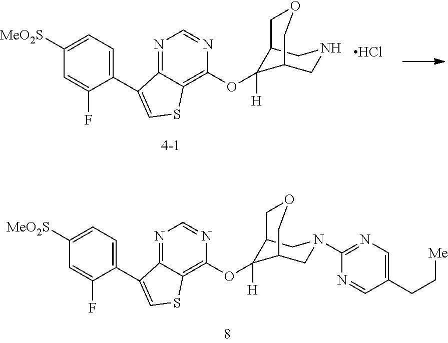 [SMILES: CS(=O)(=O)c1ccc(-c2csc3c(OC4C5CNCC4COC5)ncnc23)c(F)c1 ; Cl ; CCCc1cnc(N2CC3COCC(C2)C3Oc2ncnc3c(-c4ccc(S(C)(=O)=O)cc4F)csc23)nc1]

In similar fashion to Example 4, Step 2, Compound 4-1 was converted (heating at 110° C. 18 h) into Compound 8, a white solid, LC-MS: m/e 570 (M+1).

### Example 9

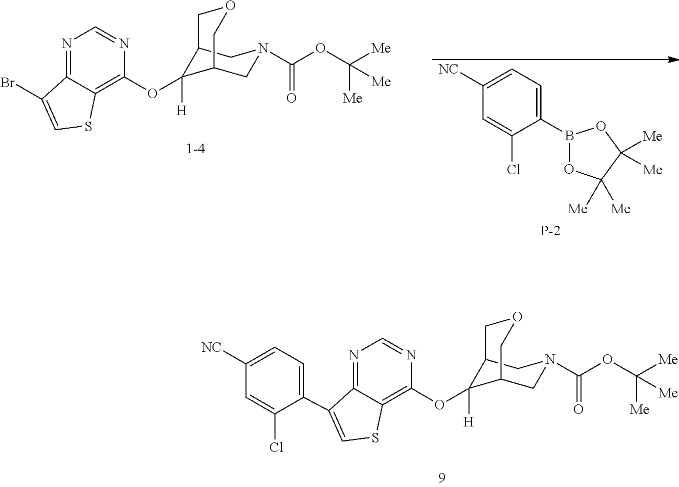

In similar fashion to Example 1, Step 3, Compound 14 was treated with Intermediate P-2 to yield Compound 9, a yellow solid, LC-MS: mile 513+515 (M+1).

### Example 10

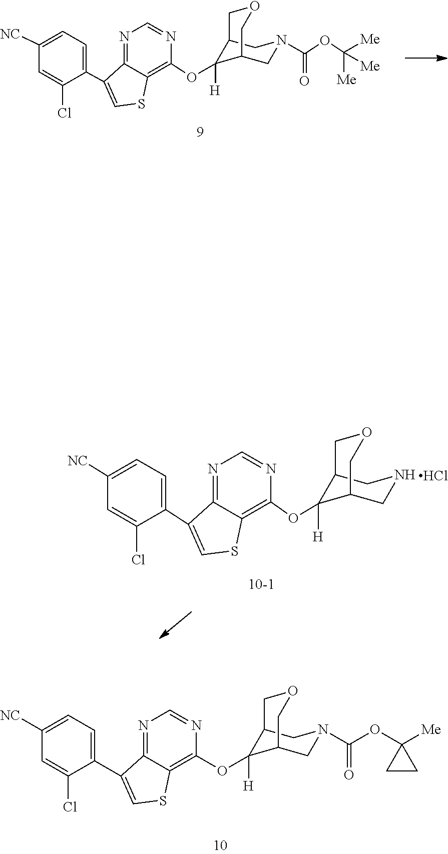 [SMILES: CC(C)(C)OC(=O)N1CC2COCC(C1)C2Oc1ncnc2c(-c3ccc(C#N)cc3Cl)csc12 ; N#Cc1ccc(-c2csc3c(OC4C5CNCC4COC5)ncnc23)c(Cl)c1 ; Cl ; CC1(OC(=O)N2CC3COCC(C2)C3Oc2ncnc3c(-c4ccc(C#N)cc4Cl)csc23)CC1]

In similar fashion to Example 3, Compound 9 was converted into Compound 10, a white solid, LC-MS: m/e 511+513 (M+1).

### Example 11

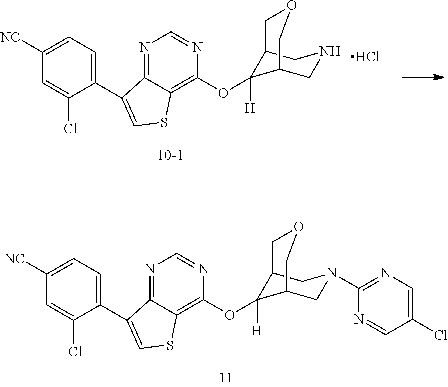 [SMILES: N#Cc1ccc(-c2csc3c(OC4C5CNCC4COC5)ncnc23)c(Cl)c1 ; Cl ; N#Cc1ccc(-c2csc3c(OC4C5COCC4CN(c4ncc(Cl)cn4)C5)ncnc23)c(Cl)c1]

In similar fashion to Example 4, Step 2, Compound 10-1 was converted (heating at 120° C. 3 h) into Compound 11, a yellow solid, LC-MS: m/e 525+527+529 (M+1).

### Example 12

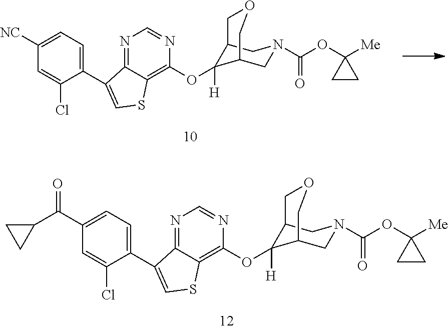 [SMILES: CC1(OC(=O)N2CC3COCC(C2)C3Oc2ncnc3c(-c4ccc(C#N)cc4Cl)csc23)CC1 ; CC1(OC(=O)N2CC3COCC(C2)C3Oc2ncnc3c(-c4ccc(C(=O)C5CC5)cc4Cl)csc23)CC1]

To Compound 10 (0.018 g, 0.035 mmol) in THF (1.0 mL) was added cyclopropylmagnesium bromide (0.5M in THF, 0.28 mL, 0.14 mmol). The mixture was heated at 60° C. 18 h, allowed to cool, and treated with water (1 mL). Concentration and purification by PLC yielded Compound 12 as a yellow solid, LC-MS: m/e 554+556 (M+1).

### Example 13

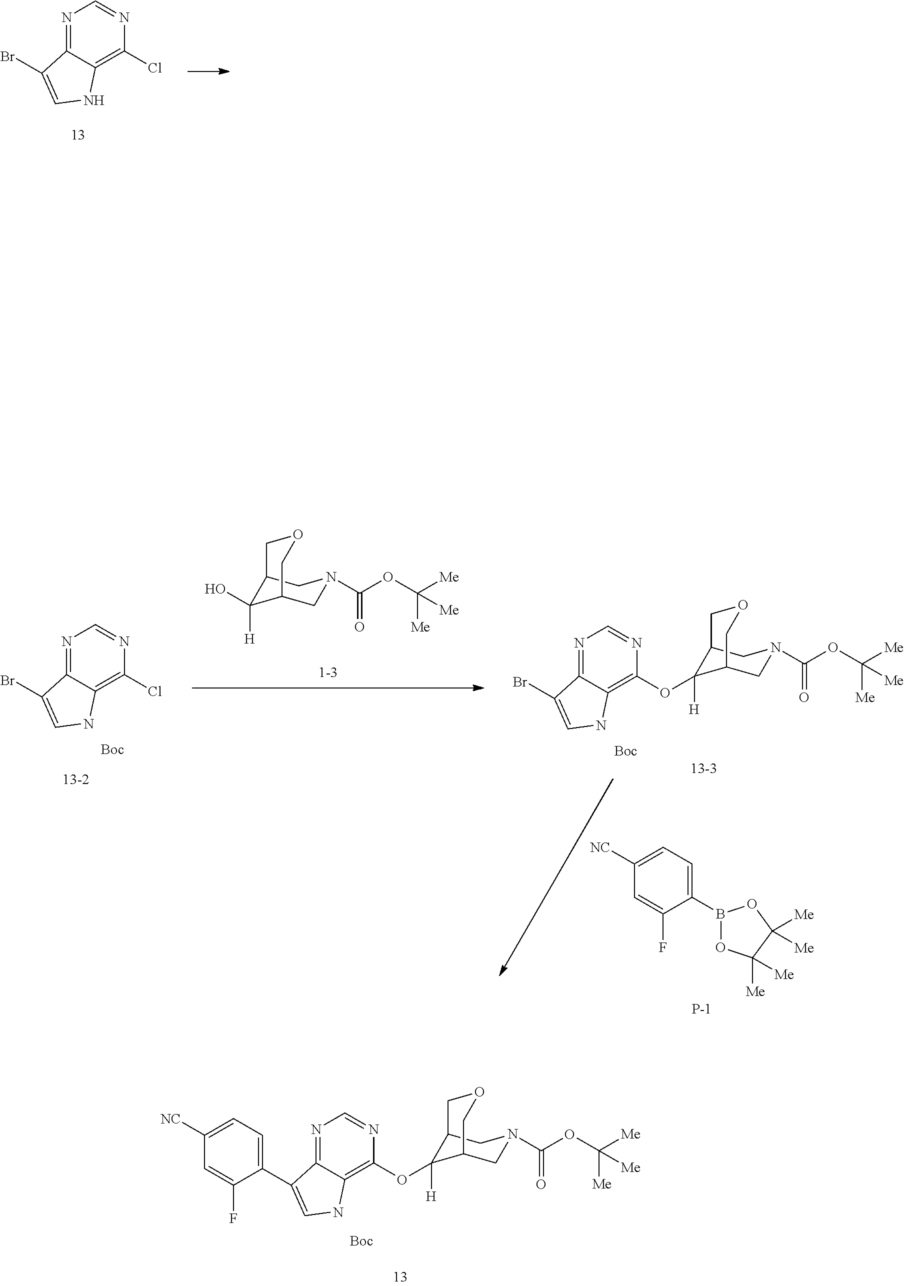

Step 1: To Compound 13-1 (0.50 g, 2.2 mmol) in THF (15 mL) were added di-t-butyl dicarbonate (0.56 g, 2.6 mmol) and 4-(dimethylamino)pyridine. (0.026 g, 0.21 mmol). The mixture was stirred 18 h and concentrated to provide crude Compound 13-2,

Step 2: The material from Step 1 was dissolved in DMF (5 mL) and Compound 1-3 (0.52 g, 2.1 mmol) and NaH (0.22 g, 60% in oil, 5.4 mmol) were added. The mixture was heated at 40° C. 64 h and concentrated. Purification by PLC (3% MeOH/CH₂Cl₂) yielded Compound 13-3 as a yellow solid.

Step 3: In similar fashion to Example 1, Step 3, Compound 13-3 was converted into Compound 13, a white solid, LC-MS: m/e 580 (M+1).

### Example 14

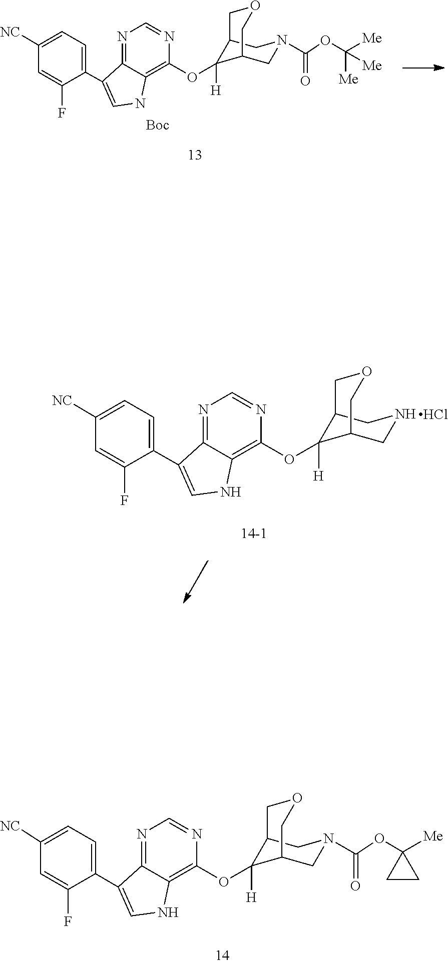 [SMILES: CC(C)(C)OC(=O)N1CC2COCC(C1)C2Oc1ncnc2c(-c3ccc(C#N)cc3F)c[nH]c12 ; N#Cc1ccc(-c2c[nH]c3c(OC4C5CNCC4COC5)ncnc23)c(F)c1 ; Cl ; CC1(OC(=O)N2CC3COCC(C2)C3Oc2ncnc3c(-c4ccc(C#N)cc4F)c[nH]c23)CC1]

Compound 13 was converted to Compound 14-1 according to the procedure of Example 3, Step 1, with heating at 40° C. for 40 min. Treatment of Compound 14-1 according to Step 2 of Example 3 yielded Compound 14 as a white solid, LC-MS: m/e 478 (M+1).

### Example 15

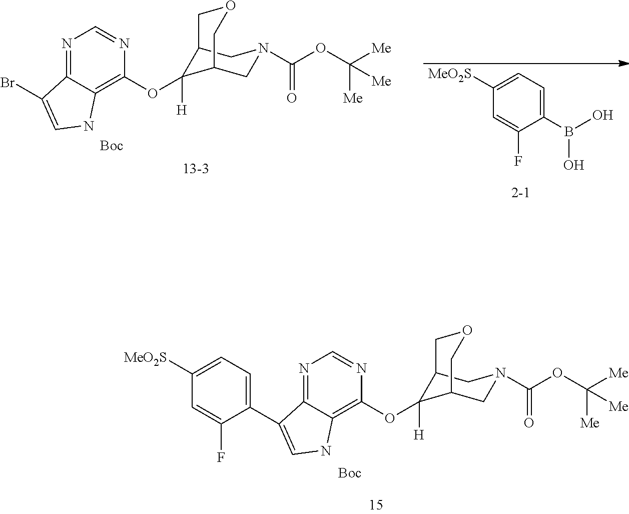 [SMILES: CC(C)(C)OC(=O)N1CC2COCC(C1)C2Oc1ncnc2c(Br)c[nH]c12 ; CS(=O)(=O)c1ccc(B(O)O)c(F)c1 ; CC(C)(C)OC(=O)N1CC2COCC(C1)C2Oc1ncnc2c(-c3ccc(S(C)(=O)=O)cc3F)c[nH]c12]

In similar fashion to Example 1, Step 3, Compound 13-3 was converted into Compound 15, a white solid.

### Example 16

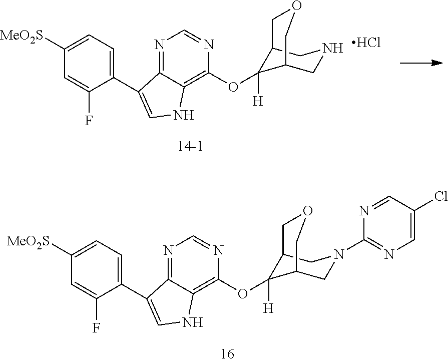 [SMILES: CS(=O)(=O)c1ccc(-c2c[nH]c3c(OC4C5CNCC4COC5)ncnc23)c(F)c1 ; Cl ; CS(=O)(=O)c1ccc(-c2c[nH]c3c(OC4C5COCC4CN(c4ncc(Cl)cn4)C5)ncnc23)c(F)c1]

In similar fashion to Example 4, Step 2, Compound 14-1 was converted into Compound 16, a white solid, LC-MS: m/e 545+547 (M+1).

### Example 17

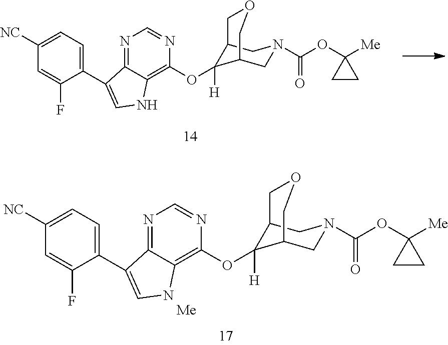 [SMILES: CC1(OC(=O)N2CC3COCC(C2)C3Oc2ncnc3c(-c4ccc(C#N)cc4F)c[nH]c23)CC1 ; CC1(OC(=O)N2CC3COCC(C2)C3Oc2ncnc3c(-c4ccc(C#N)cc4F)c[nH]c23)CC1]

To Compound 14 (0.025 g, 0.052 mmol) in THF (1.0 mL) were added CH₃I (0.010 mL, 0.16 mmol) and NaH (60% in oil, 0.007 g, 0.18 mmol). The mixture was stirred 18 h, concentrated, and purified by PLC to yield Compound 17 as a white solid, LC-MS: m/e 492 (M+1).

### Example 18

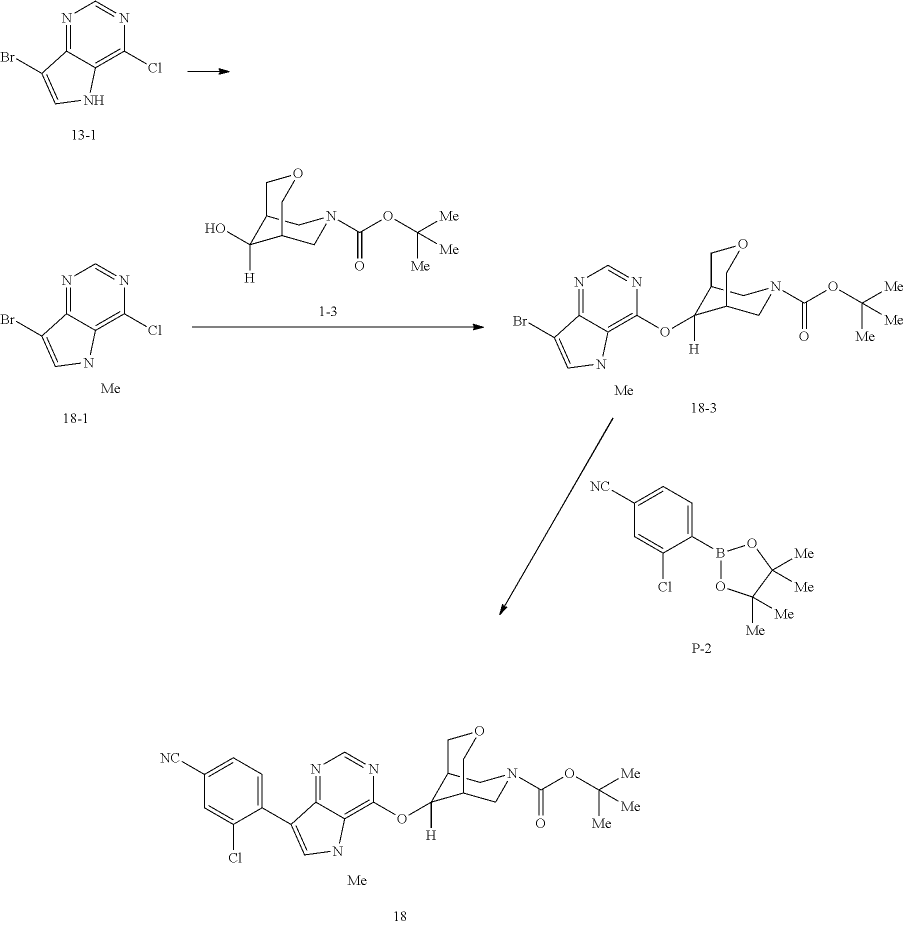

Step 1: To Compound 134 (0.30 g, 1.3 mmol) and CH₃I (0.12 mL, 1.9 mmol) in THF (5 mL) was added NaH (60% in oil, 0.078 g, 1.9 mmol). The mixture was stirred 18 h and concentrated.

Step 2: The material from Step 1 was combined with Compound 1-3 (0.38 g, 1.6 mmol) and NaO-t-Bu (0.19 g, 1.9 mmol) in dioxane (8 mL) and the mixture heated at 100° C. 3 h, allowed to cool, and concentrated. Chromatography on silica (3% MeOH/CH₂Cl₂) gave Compound 18-2 as a yellow solid.

Step 3: In similar fashion to Example 1, Step 3, Compound 18-2 was converted into Compound 18, a yellow solid.

### Example 19

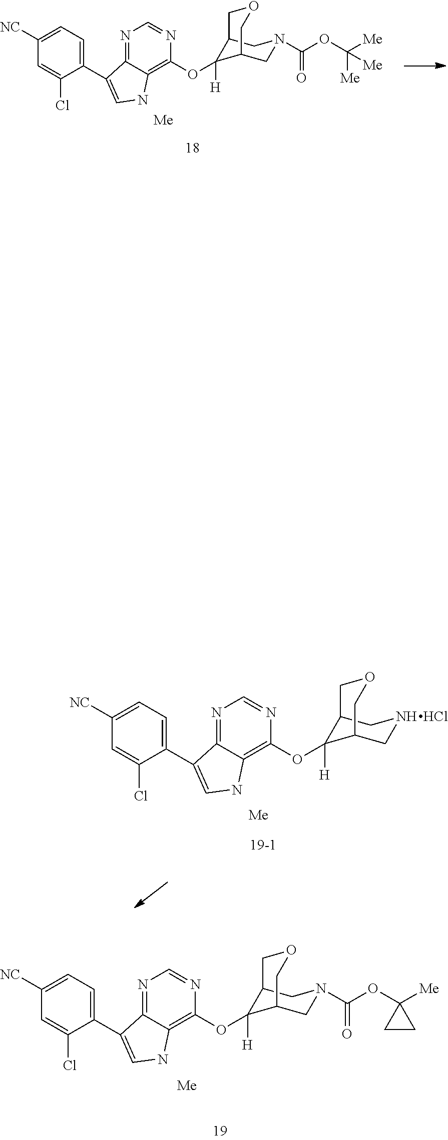 [SMILES: CC(C)(C)OC(=O)N1CC2COCC(C1)C2Oc1ncnc2c(-c3ccc(C#N)cc3Cl)c[nH]c12 ; N#Cc1ccc(-c2c[nH]c3c(OC4C5CNCC4COC5)ncnc23)c(Cl)c1 ; Cl ; CC1(OC(=O)N2CC3COCC(C2)C3Oc2ncnc3c(-c4ccc(C#N)cc4Cl)c[nH]c23)CC1]

Compound 18 was converted to Compound 19 according to the procedures of Example 3. Purification by PLC (25% EtOAc/CH₂Cl₂) yielded Compound 19 as a yellow solid, LC-MS: rule 508+510 (M+1).

### Example 20

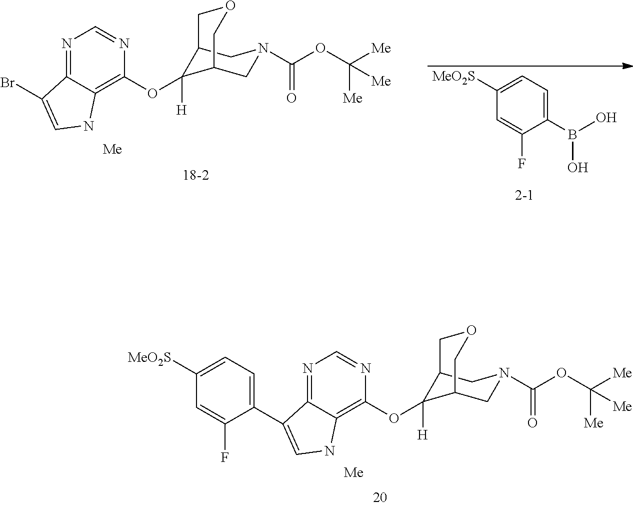 [SMILES: CC(C)(C)OC(=O)N1CC2COCC(C1)C2Oc1ncnc2c(Br)c[nH]c12 ; CS(=O)(=O)c1ccc(B(O)O)c(F)c1 ; CC(C)(C)OC(=O)N1CC2COCC(C1)C2Oc1ncnc2c(-c3ccc(S(C)(=O)=O)cc3F)c[nH]c12]

In similar fashion to Example 2, Compound 18-2 was converted into Compound 20, a yellow solid.

### Example 21

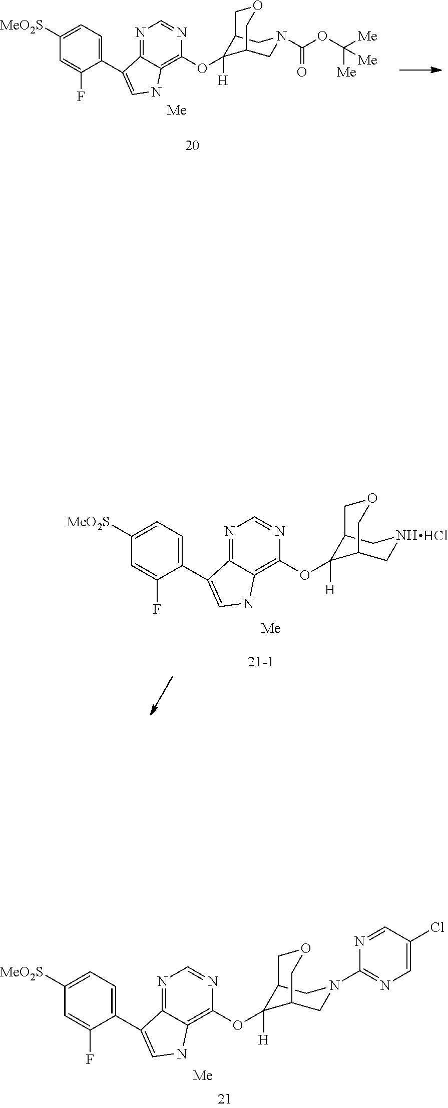 [SMILES: CC(C)(C)OC(=O)N1CC2COCC(C1)C2Oc1ncnc2c(-c3ccc(S(C)(=O)=O)cc3F)c[nH]c12 ; CS(=O)(=O)c1ccc(-c2c[nH]c3c(OC4C5CNCC4COC5)ncnc23)c(F)c1 ; Cl ; CS(=O)(=O)c1ccc(-c2c[nH]c3c(OC4C5COCC4CN(c4ncc(Cl)cn4)C5)ncnc23)c(F)c1]

In similar fashion to Example 4, Compound 20 was converted into Compound 21, a yellow solid, LC-MS: m/e 559+561 (M+1).

### Example 22

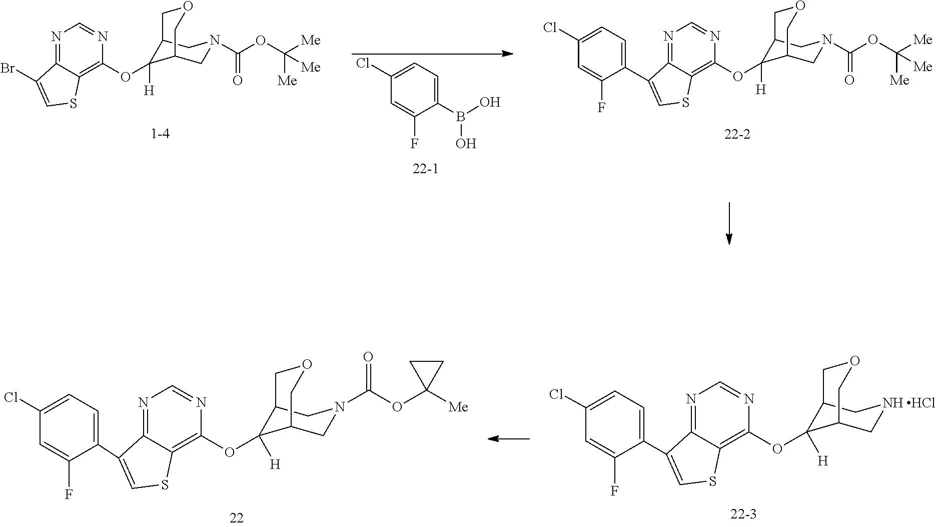 [SMILES: CC(C)(C)OC(=O)N1CC2COCC(C1)C2Oc1ncnc2c(Br)csc12 ; OB(O)c1ccc(Cl)cc1F ; CC(C)(C)OC(=O)N1CC2COCC(C1)C2Oc1ncnc2c(-c3ccc(Cl)cc3F)csc12 ; CC1(OC(=O)N2CC3COCC(C2)C3Oc2ncnc3c(-c4ccc(Cl)cc4F)csc23)CC1 ; Fc1cc(Cl)ccc1-c1csc2c(OC3C4CNCC3COC4)ncnc12 ; Cl]

Step 1: In similar fashion to Example 1, Step 3, Compound 1-4 was converted to Compound 22-2.

Steps 2 and 3. In similar fashion to Example 3, Compound 22-2 was converted to Compound 22, a yellow solid, LC-MS: m/e 504+506 (M+1).

### Example 23

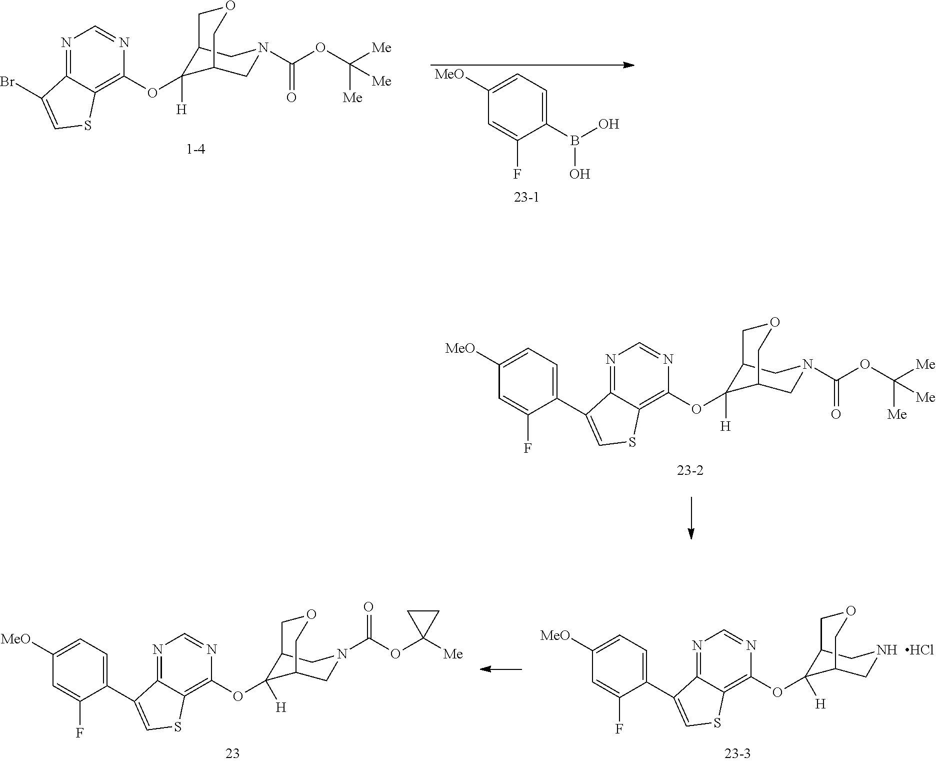 [SMILES: CC(C)(C)OC(=O)N1CC2COCC(C1)C2Oc1ncnc2c(Br)csc12 ; COc1ccc(B(O)O)c(F)c1 ; COc1ccc(-c2csc3c(OC4C5COCC4CN(C(=O)OC(C)(C)C)C5)ncnc23)c(F)c1 ; COc1ccc(-c2csc3c(OC4C5COCC4CN(C(=O)OC4(C)CC4)C5)ncnc23)c(F)c1 ; COc1ccc(-c2csc3c(OC4C5CNCC4COC5)ncnc23)c(F)c1 ; Cl]

Step 1: In similar fashion to Example 1, Step 3, Compound 1-4 was converted to Compound 23-2.

Steps 2 and 3. In similar fashion to Example 3, Compound 23-2 was converted to Compound 23, a yellow solid, LC-MS: m/e 500 (M+1).

### Example 24

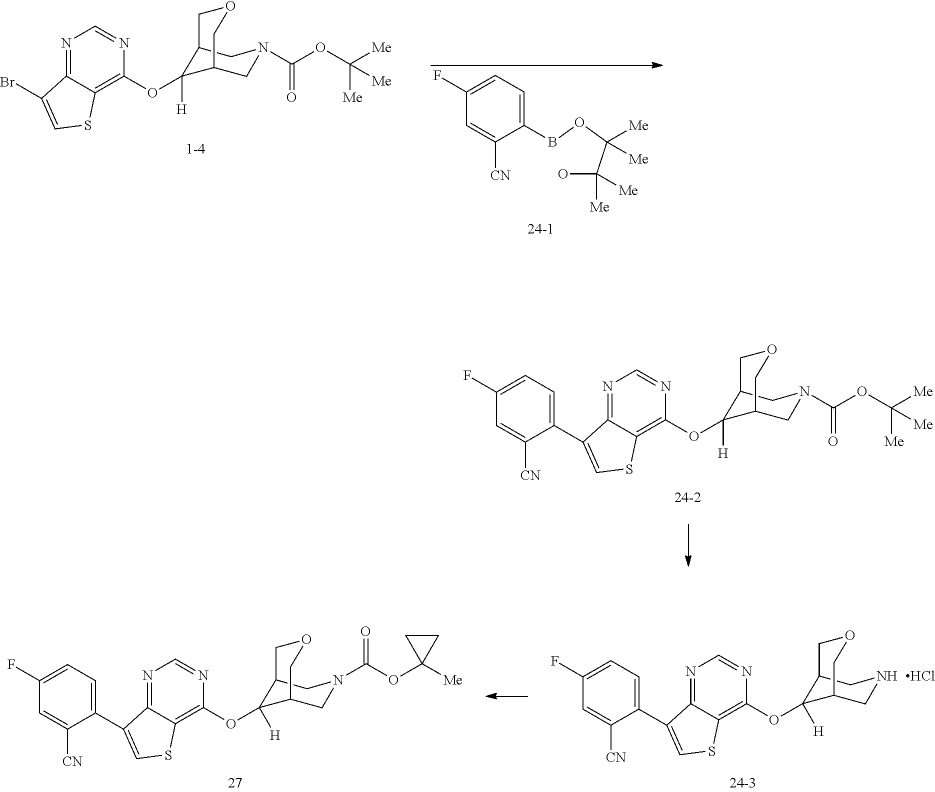

Step 1: In similar fashion to Example 1, Step 3, Compound 1-4 was converted to Compound 24-2.

Steps 2 and 3. In similar fashion to Example 3, Compound 24-2 was converted to Compound 24, a white solid, LC-MS: m/e 495 (M+1).

### Example 25

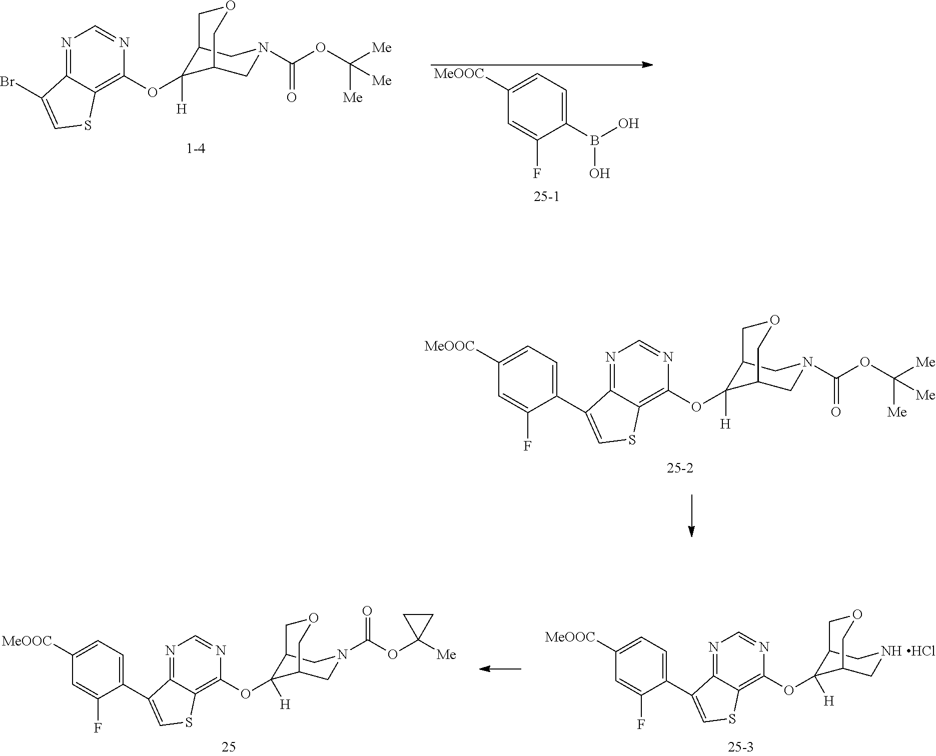 [SMILES: CC(C)(C)OC(=O)N1CC2COCC(C1)C2Oc1ncnc2c(Br)csc12 ; COC(=O)c1ccc(B(O)O)c(F)c1 ; COC(=O)c1ccc(-c2csc3c(OC4C5COCC4CN(C(=O)OC(C)(C)C)C5)ncnc23)c(F)c1 ; COC(=O)c1ccc(-c2csc3c(OC4C5COCC4CN(C(=O)OC4(C)CC4)C5)ncnc23)c(F)c1 ; COC(=O)c1ccc(-c2csc3c(OC4C5CNCC4COC5)ncnc23)c(F)c1 ; Cl]

Step 1: In similar fashion to Example 1, Step 3, Compound 1-4 was converted to Compound 25-2.

Steps 2 and 3. In similar fashion to Example 3, Compound 25-2 was converted to Compound 25, a white solid, LC-MS: m/e 528 (M+1).

### Example 26

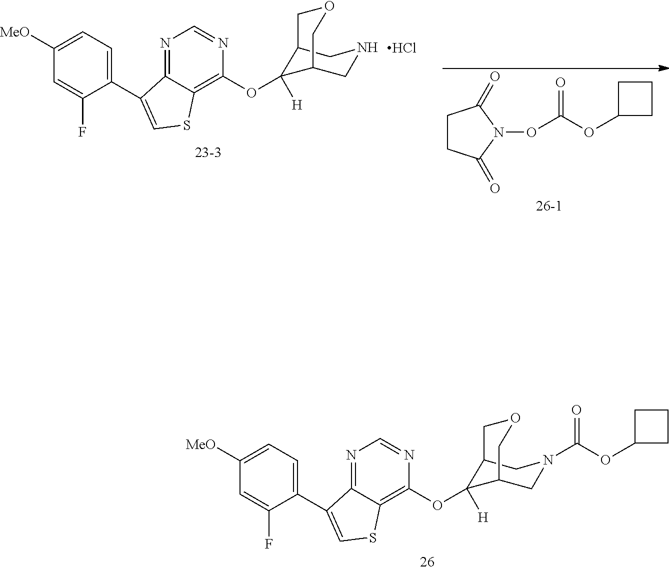 [SMILES: COc1ccc(-c2csc3c(OC4C5CNCC4COC5)ncnc23)c(F)c1 ; Cl ; O=C(OC1CCC1)ON1C(=O)CCC1=O ; COc1ccc(-c2csc3c(OC4C5COCC4CN(C(=O)OC4CCC4)C5)ncnc23)c(F)c1]

In similar fashion to Example 3, Step 2, Compound 23-3 was treated with Compound 26-1 to provide Compound 26, a white solid, LC-MS: m/e 500 (M+1).

### Example 27

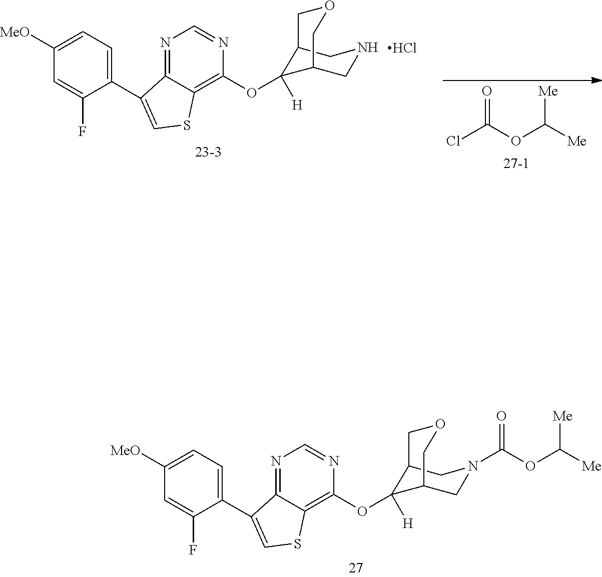 [SMILES: COc1ccc(-c2csc3c(OC4C5CNCC4COC5)ncnc23)c(F)c1 ; Cl ; CC(C)OC(=O)Cl ; COc1ccc(-c2csc3c(OC4C5COCC4CN(C(=O)OC(C)C)C5)ncnc23)c(F)c1]

In similar fashion to Example 3, Step 2, Compound 23-3 was converted to Compound 27, a white solid, LC-MS: m/e 488 (M+1).

### Example 28

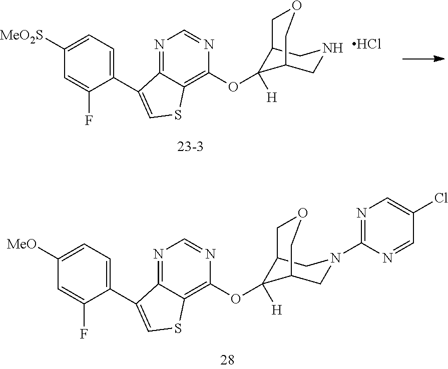 [SMILES: CS(=O)(=O)c1ccc(-c2csc3c(OC4C5CNCC4COC5)ncnc23)c(F)c1 ; Cl ; COc1ccc(-c2csc3c(OC4C5COCC4CN(c4ncc(Cl)cn4)C5)ncnc23)c(F)c1]

In similar fashion to Example 4, Step 2, Compound 23-3 was converted to Compound 28, a white solid, LC-MS: ink 514+516 (M+1).

### Example 29

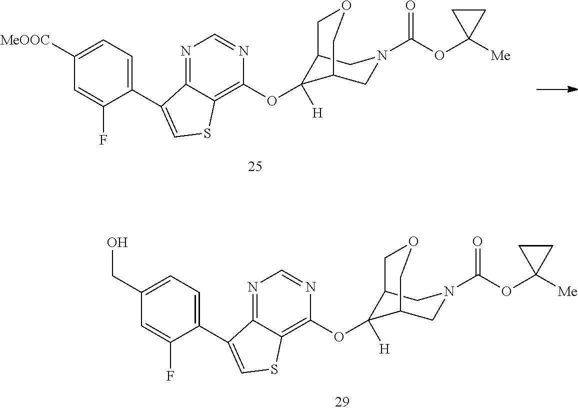 [SMILES: COC(=O)c1ccc(-c2csc3c(OC4C5COCC4CN(C(=O)OC4(C)CC4)C5)ncnc23)c(F)c1 ; CC1(OC(=O)N2CC3COCC(C2)C3Oc2ncnc3c(-c4ccc(CO)cc4F)csc23)CC1]

Compound 25 (0.20 g, 0.37 mmol) was combined with LiBH₄ (0.034 g, 1.5 mmol) in THF (4 mL) and the mixture heated at 60° C. 5 h, allowed to cool, and partitioned with EtOAc and water. Drying (MgSO₄), concentration and purification by PLC yielded Compound 29 as a white solid, LC-MS: m/e 500 (M+1).

### Example 30

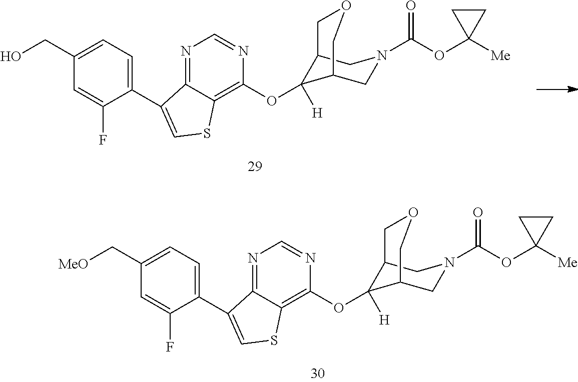 [SMILES: CC1(OC(=O)N2CC3COCC(C2)C3Oc2ncnc3c(-c4ccc(CO)cc4F)csc23)CC1 ; COCc1ccc(-c2csc3c(OC4C5COCC4CN(C(=O)OC4(C)CC4)C5)ncnc23)c(F)c1]

To Compound 29 (0.100 g, 0.20 mmol) in THF (2.0 mL) were added NaH (60% in oil, 0.016 g, 0.40 mmol) and CH₃I (0.025 mL, 0.40 mmol). The mixture was stirred 72 h and concentrated. Purification by PLC yielded Compound 30 as a white solid, LC-MS: m/e 514 (M+1).

### Example 31

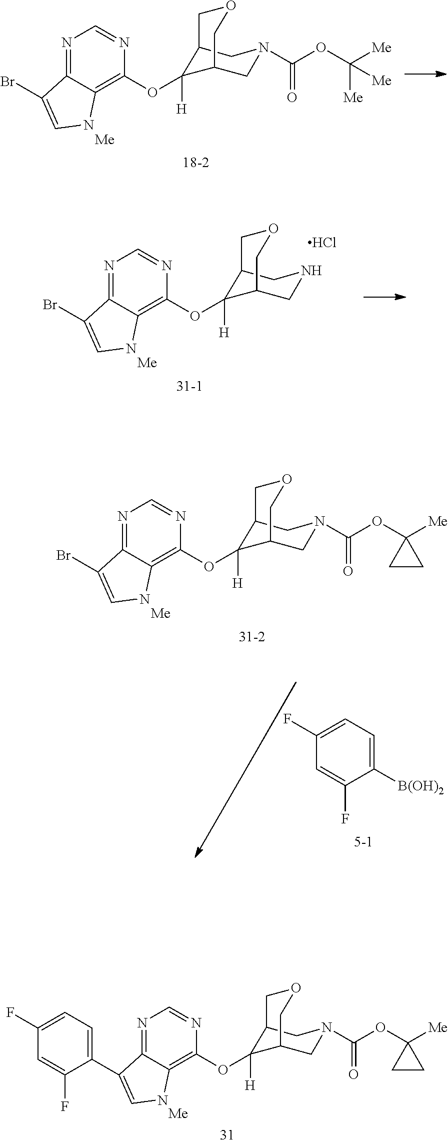 [SMILES: CC(C)(C)OC(=O)N1CC2COCC(C1)C2Oc1ncnc2c(Br)c[nH]c12 ; Brc1c[nH]c2c(OC3C4CNCC3COC4)ncnc12 ; Cl ; CC1(OC(=O)N2CC3COCC(C2)C3Oc2ncnc3c(Br)c[nH]c23)CC1 ; OB(O)c1ccc(F)cc1F ; CC1(OC(=O)N2CC3COCC(C2)C3Oc2ncnc3c(-c4ccc(F)cc4F)c[nH]c23)CC1]

Steps 1 and 2: In similar fashion to Example 3, Compound 18-2 was converted to Compound 31-2, a white solid.

Step 3: In similar fashion to Example 1, Step 3, Compound 31-2 was converted to Compound 31, a yellow solid, LC-MS: m/e 485 (M+1).

### Example 32

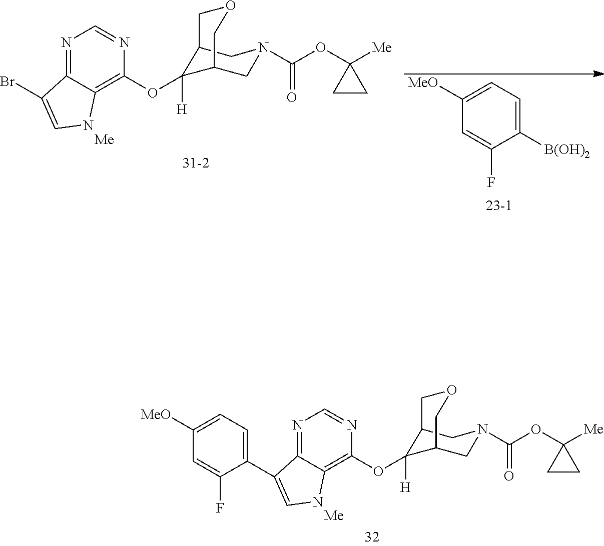 [SMILES: CC1(OC(=O)N2CC3COCC(C2)C3Oc2ncnc3c(Br)c[nH]c23)CC1 ; COc1ccc(B(O)O)c(F)c1 ; COc1ccc(-c2c[nH]c3c(OC4C5COCC4CN(C(=O)OC4(C)CC4)C5)ncnc23)c(F)c1]

In similar fashion to Example 1, Step 3, Compound 31-2 was converted to Compound 32, a yellow solid, LC-MS: m/e 497 (M+1).

### Example 33

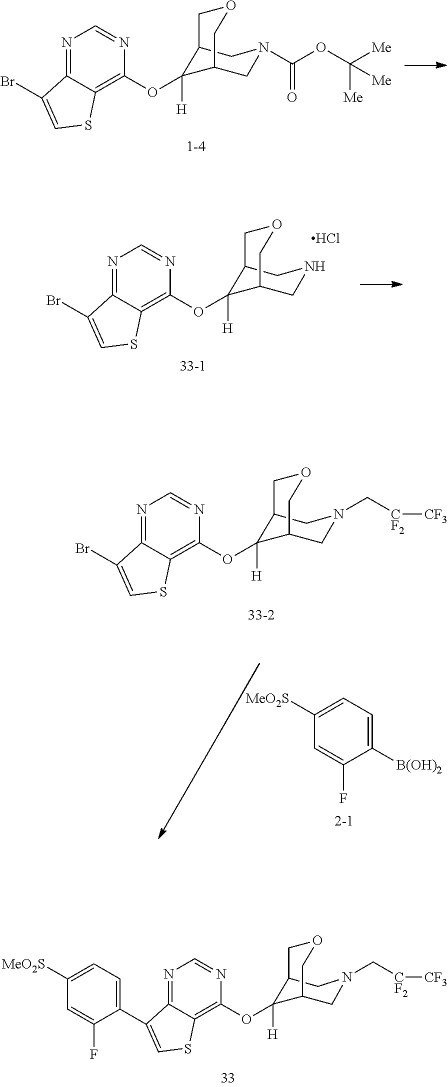 [SMILES: CC(C)(C)OC(=O)N1CC2COCC(C1)C2Oc1ncnc2c(Br)csc12 ; Brc1csc2c(OC3C4CNCC3COC4)ncnc12 ; Cl ; FC(F)(F)C(F)(F)CN1CC2COCC(C1)C2Oc1ncnc2c(Br)csc12 ; CS(=O)(=O)c1ccc(B(O)O)c(F)c1 ; CS(=O)(=O)c1ccc(-c2csc3c(OC4C5COCC4CN(CC(F)(F)C(F)(F)F)C5)ncnc23)c(F)c1]

Step 1: In similar fashion to Example 3, Step 1, Compound 1-4 (0.60 g, 1.3 mmol) was converted to Compound 33-1, a white solid.

Step 2: To the crude material from Step 1 in CH₂Cl₂ (15 mL) were added Et₃N (0.92 mL, 6.6 mmol) and 2,2,3,3,3-pentafluoroethyl trifluoromethanesulfonate (0.74 g, 2.6 mmol) and the mixture stirred 18 h. Concentration and purification by PLC yielded Compound 33-2 as a white solid,

Step 3: In similar fashion to Example 1, Step 3, Compound 33-2 was converted to Compound 33, a yellow solid, LC-MS: m/e 582 (M+1).

### Example 34

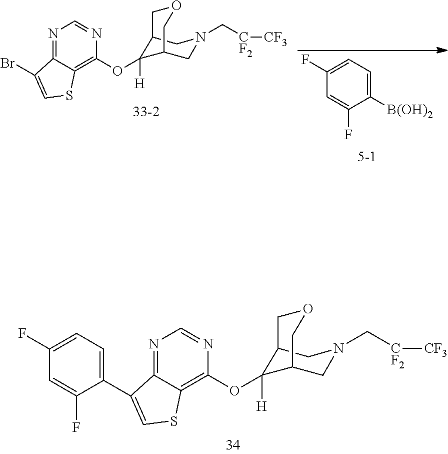 [SMILES: FC(F)(F)C(F)(F)CN1CC2COCC(C1)C2Oc1ncnc2c(Br)csc12 ; OB(O)c1ccc(F)cc1F ; Fc1ccc(-c2csc3c(OC4C5COCC4CN(CC(F)(F)C(F)(F)F)C5)ncnc23)c(F)c1]

In similar fashion to Example 1, Step 3, Compound 33-2 was converted to Compound 34, a white solid, LC-MS: m/e 522 (M+1).

### Example 35

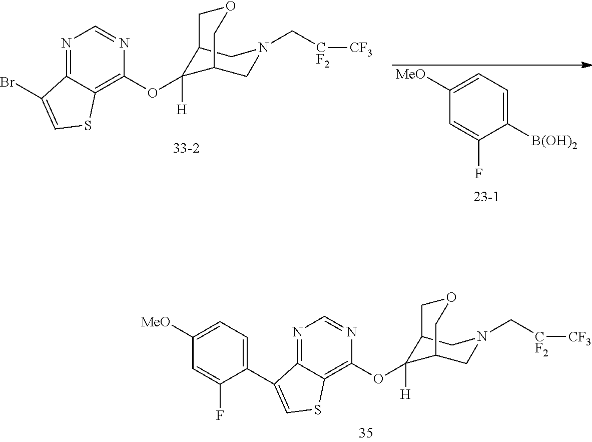 [SMILES: FC(F)(F)C(F)(F)CN1CC2COCC(C1)C2Oc1ncnc2c(Br)csc12 ; COc1ccc(B(O)O)c(F)c1 ; COc1ccc(-c2csc3c(OC4C5COCC4CN(CC(F)(F)C(F)(F)F)C5)ncnc23)c(F)c1]

In similar fashion to Example 1, Step 3, Compound 33-2 was converted to Compound 35, a white solid, LC-MS: m/e 534 (M+1).

### Example 36

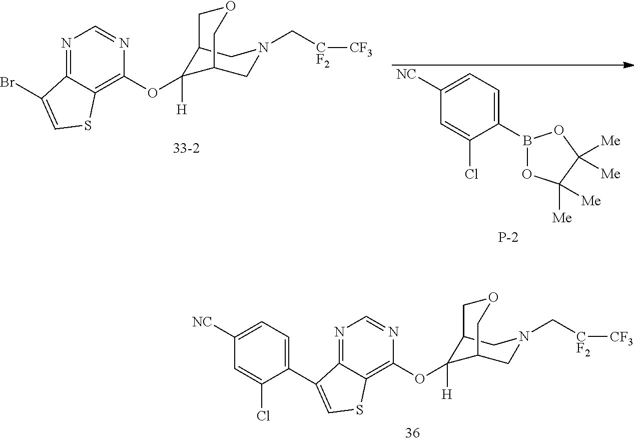

In similar fashion to Example 1, Step 3, Compound 33-2 was converted to Compound 36, a white solid, LC-MS: m/e 545, 547 (M+1).

### Example 37

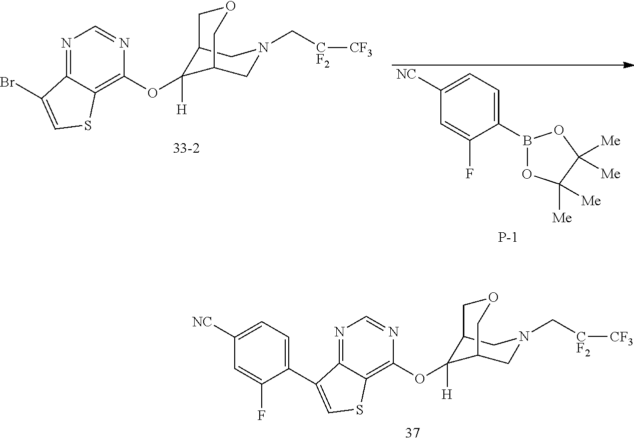

In similar fashion to Example 1, Step 3, Compound 33-2 was converted to Compound 37, a white solid, LC-MS: m/e 529 (M+1).

### Example 38

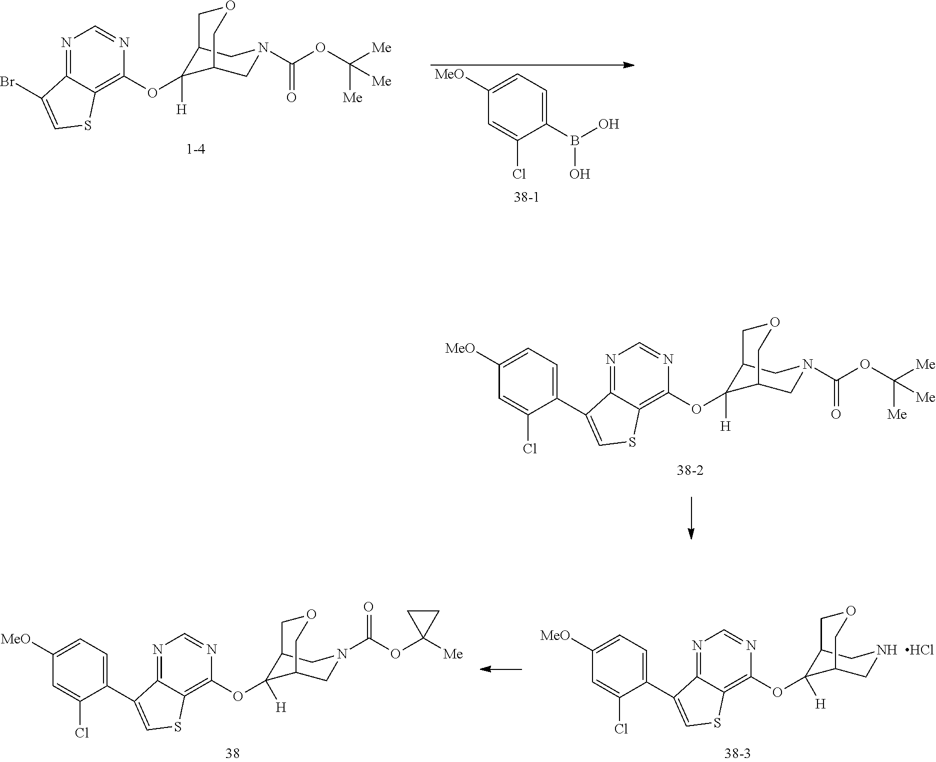 [SMILES: CC(C)(C)OC(=O)N1CC2COCC(C1)C2Oc1ncnc2c(Br)csc12 ; COc1ccc(B(O)O)c(Cl)c1 ; COc1ccc(-c2csc3c(OC4C5COCC4CN(C(=O)OC(C)(C)C)C5)ncnc23)c(Cl)c1 ; COc1ccc(-c2csc3c(OC4C5COCC4CN(C(=O)OC4(C)CC4)C5)ncnc23)c(Cl)c1 ; COc1ccc(-c2csc3c(OC4C5CNCC4COC5)ncnc23)c(Cl)c1 ; Cl]

Step 1: In similar fashion to Example 1, Step 3, Compound 1-4 was converted to Compound 38-2.

Steps 2 and 3. In similar fashion to Example 3, Compound 38-2 was converted to Compound 38, a yellow solid, LC-MS: m/e 516+518 (M+1).

### Example 39

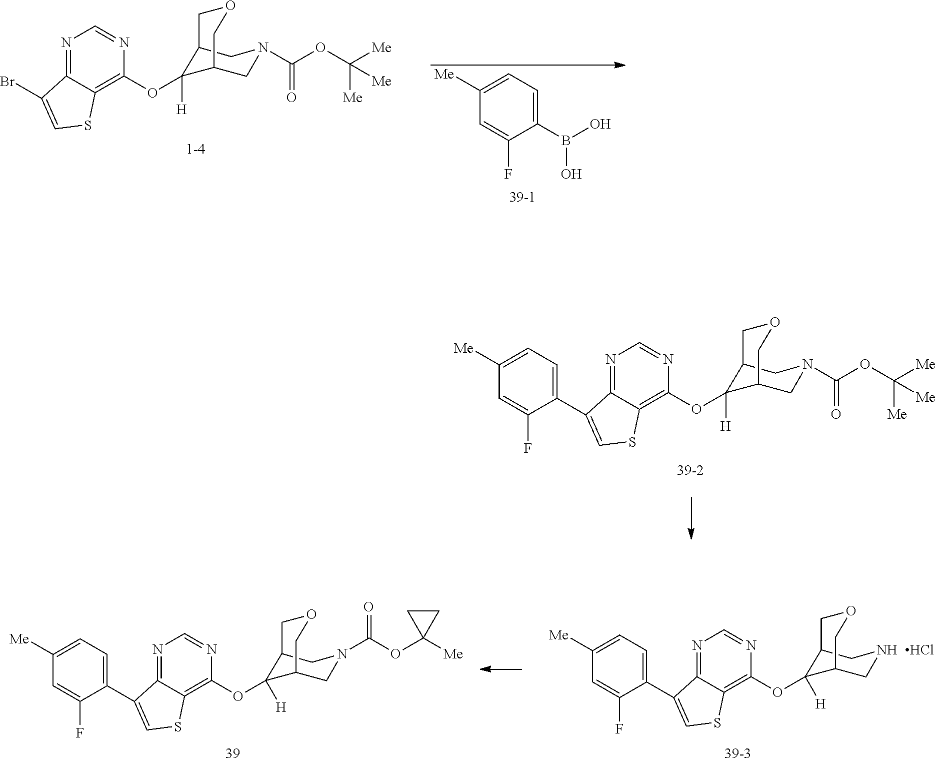 [SMILES: CC(C)(C)OC(=O)N1CC2COCC(C1)C2Oc1ncnc2c(Br)csc12 ; Cc1ccc(B(O)O)c(F)c1 ; Cc1ccc(-c2csc3c(OC4C5COCC4CN(C(=O)OC(C)(C)C)C5)ncnc23)c(F)c1 ; Cc1ccc(-c2csc3c(OC4C5COCC4CN(C(=O)OC4(C)CC4)C5)ncnc23)c(F)c1 ; Cc1ccc(-c2csc3c(OC4C5CNCC4COC5)ncnc23)c(F)c1 ; Cl]

Step 1: In similar fashion to Example 1, Step 3, Compound 1-4 was converted to Compound 39-2.

Steps 2 and 3. In similar fashion to Example 3, Compound 39-2 was converted to Compound 39, a yellow solid, LC-MS: m/e 484 (M+1).

### Example 40

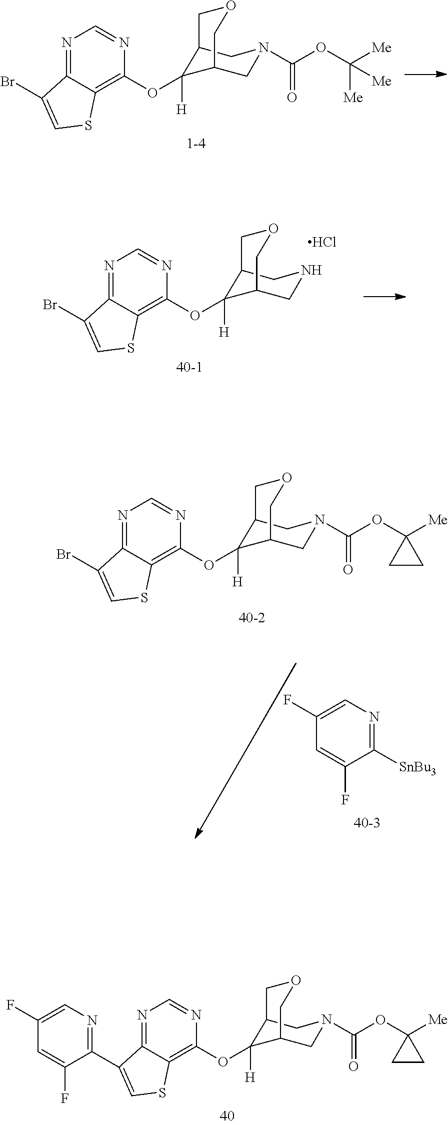

Steps 1 and 2: In similar fashion to Example 3, Compound 1-4 was converted to Compound 41, a yellow solid.

Step 3: Compound 40-2 (0.090 g, 0.20 mmol) was combined with Compound 40-3 (0.096 g, 0.24 mmol) and Pd(dppf)Cl₂ (0.014 g, 0.021=01) in toluene (1.5 mL). The mixture was heated by microwave at 100° C. 2 h, concentrated, and purified by PLC to yield Compound 40 as a yellow solid, LC-MS: m/e 489 (M+1).

### Example 41

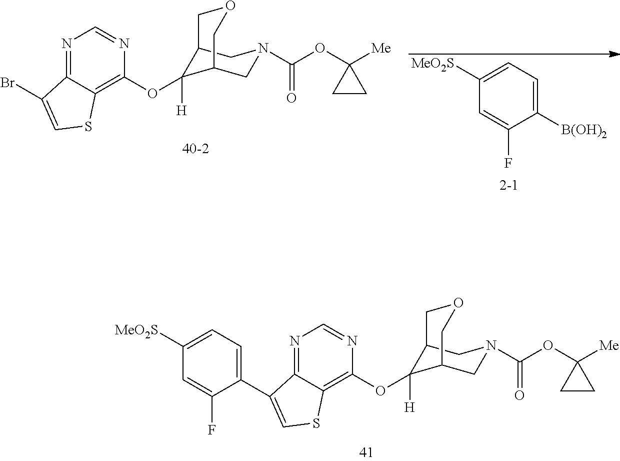 [SMILES: CC1(OC(=O)N2CC3COCC(C2)C3Oc2ncnc3c(Br)csc23)CC1 ; CS(=O)(=O)c1ccc(B(O)O)c(F)c1 ; CC1(OC(=O)N2CC3COCC(C2)C3Oc2ncnc3c(-c4ccc(S(C)(=O)=O)cc4F)csc23)CC1]

In similar fashion to Example 1, Step 3, Compound 40-2 was converted to Compound 40, a yellow solid, LC-MS: m/e 548 (M+1).

### Example 42

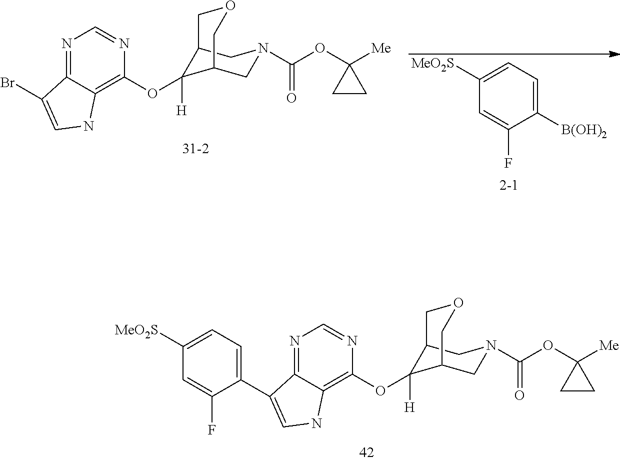 [SMILES: CC1(OC(=O)N2CC3COCC(C2)C3Oc2ncnc3c(Br)c[nH]c23)CC1 ; CS(=O)(=O)c1ccc(B(O)O)c(F)c1 ; CC1(OC(=O)N2CC3COCC(C2)C3Oc2ncnc3c(-c4ccc(S(C)(=O)=O)cc4F)c[nH]c23)CC1]

In similar fashion to Example 1, Step 3, Compound 31-2 was converted to Compound 42, a yellow solid, LC-MS: m/e 545 (M+1).

### Example 43

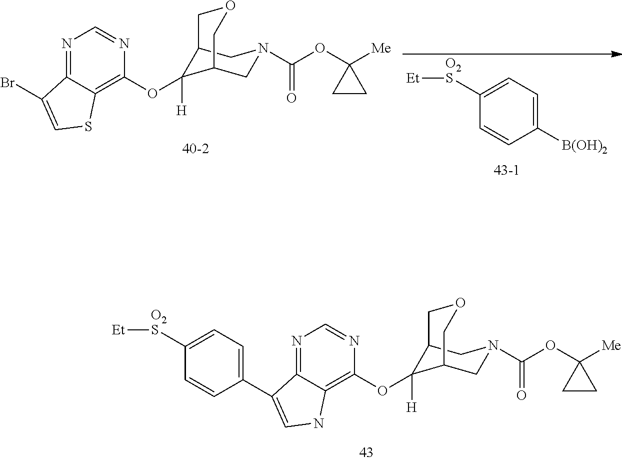 [SMILES: CC1(OC(=O)N2CC3COCC(C2)C3Oc2ncnc3c(Br)csc23)CC1 ; CCS(=O)(=O)c1ccc(B(O)O)cc1 ; CCS(=O)(=O)c1ccc(-c2c[nH]c3c(OC4C5COCC4CN(C(=O)OC4(C)CC4)C5)ncnc23)cc1]

In similar fashion to Example 1, Step 3, Compound 40-2 was converted to Compound 43, a yellow solid, LC-MS: m/e 544 (M+1).

### Example 44

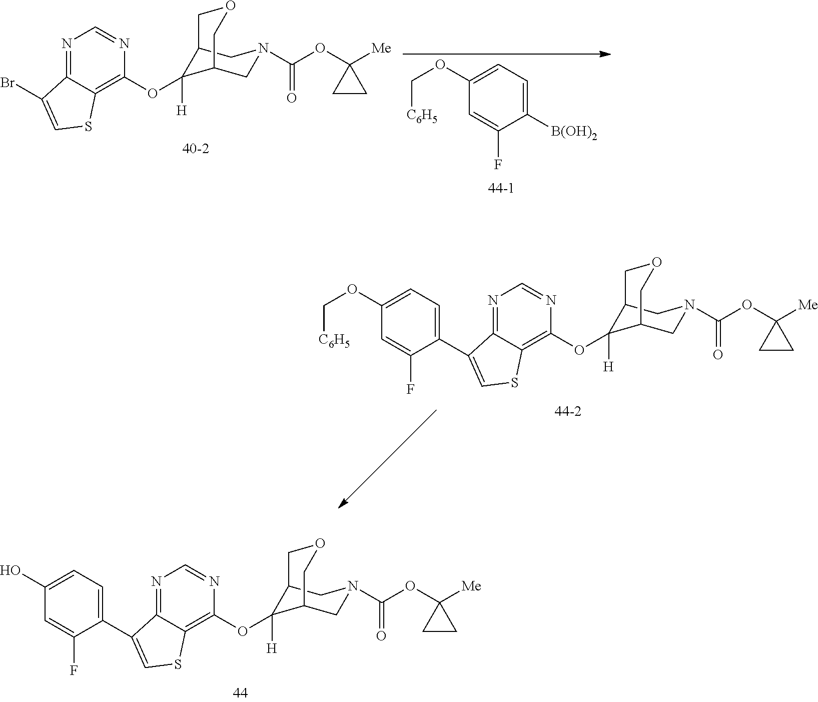

Step 1: In similar fashion to Example 1, Step 3, Compound 40-2 was converted to Compound 44-2, a yellow solid.

Step 2: Compound 44-2 (0.13 g, 0.23 mmol) was combined with 10% Pd/C (0.10 g) in DOH (2.5 mL) and EtOAc (2.5 mL) and hydrogenated under balloon pressure for 64 h. Filtration and concentration yielded Compound 44, a yellow solid, LC-MS: rn/e 486 (M+1).

### Example 45

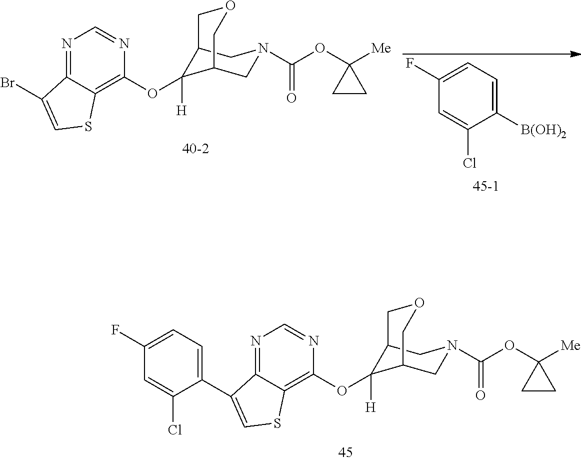

In similar fashion to Example 1, Step 3, Compound 40-2 was converted to Compound 45, a white solid, LC-MS: m/e 504+506 (M+1).

### Example 46

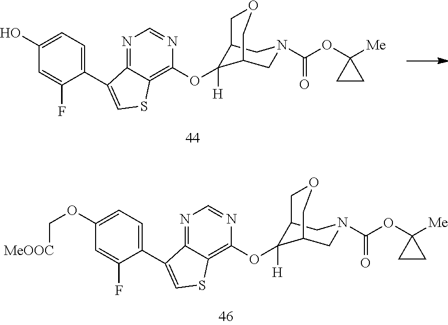 [SMILES: CC1(OC(=O)N2CC3COCC(C2)C3Oc2ncnc3c(-c4ccc(O)cc4F)csc23)CC1 ; COC(=O)COc1ccc(-c2csc3c(OC4C5COCC4CN(C(=O)OC4(C)CC4)C5)ncnc23)c(F)c1]

Compound 44 (0.075 g, 0.15 mmol) was combined with methyl bromoacetate (0.028 mL, 0.30 mmol) and K₂CO₃ (0.053 g, 0.38 mmol) in DMF (1.0 mL) and heated at 100° C. 18 h. Concentration and purification by PLC (4% MeOH/CH₂Cl₂) yielded Compound 46, a yellow solid, LC-MS: m/e 558 (M+1).

### Example 47

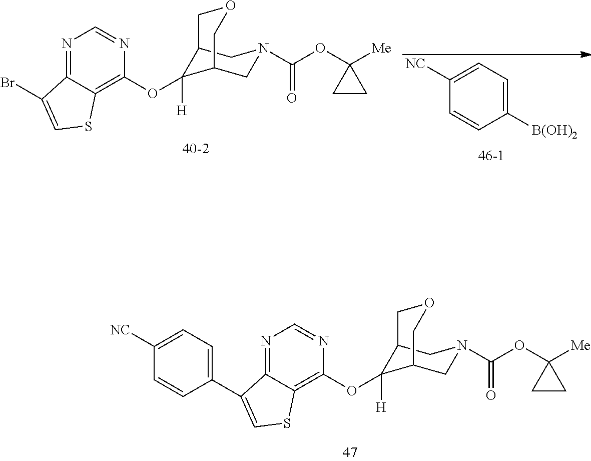

In similar fashion to Example 1, Step 3, Compound 40-2 was converted to Compound 47, a white solid, LC-MS: m/e 477 (M+1).

### Example 48

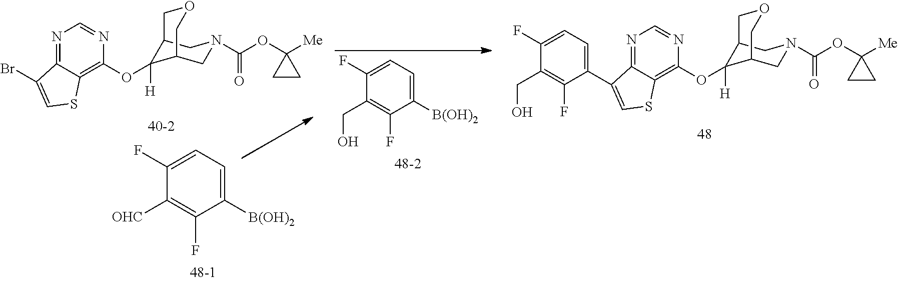 [SMILES: CC1(OC(=O)N2CC3COCC(C2)C3Oc2ncnc3c(Br)csc23)CC1 ; OCc1c(F)ccc(B(O)O)c1F ; CC1(OC(=O)N2CC3COCC(C2)C3Oc2ncnc3c(-c4ccc(F)c(CO)c4F)csc23)CC1 ; O=Cc1c(F)ccc(B(O)O)c1F]

Step 1: Compound 48-1 (0.311 g, 1.67 mmol) was dissolved in THF/MeOH (1:1, 8 mL). NaBH₄ (0.0995 g, 2.5 mmol) was added. After 4 h, saturated NH₄Cl and 1.0N HCl were added to pH 2-3. Extraction with ether, drying (MgSO₄), and concentration left Compound 48-2 as a white solid.

Step 2: In similar fashion to Example 1, Step 3, Compound 40-2 was treated with Compound 48-2 to yield Compound 48, a yellow solid, LC-MS: in/e 518 (M+1).

### Example 49

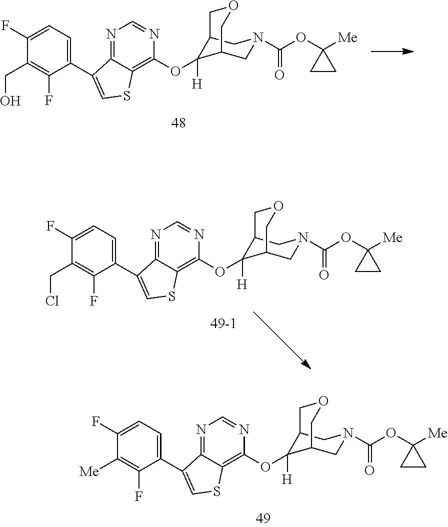 [SMILES: CC1(OC(=O)N2CC3COCC(C2)C3Oc2ncnc3c(-c4ccc(F)c(CO)c4F)csc23)CC1 ; CC1(OC(=O)N2CC3COCC(C2)C3Oc2ncnc3c(-c4ccc(F)c(CCl)c4F)csc23)CC1 ; Cc1c(F)ccc(-c2csc3c(OC4C5COCC4CN(C(=O)OC4(C)CC4)C5)ncnc23)c1F]

Step 1: Compound 48 (0.095 g, 0.18 mmol) was dissolved in CH₂Cl₂ (2 mL), and SOCl₂ (0.053 mL, 0.7 mmol) was added. The mixture was heated at reflux 0.5 h and concentrated to provide crude Compound 49-1.

Step 2: The material from Step 1 was combined with 10% Pd/C (0.05 g) and ammonium formate (0.050 g, 0.8 mmol). The mixture was heated at 75° C. 2 h, allowed to cool, filtered, and concentrated. Purification by PLC (5% acetone/CH₂Cl₂) yielded Compound 49, a white solid, LC-MS: ink 502 (M+1).

### Example 50

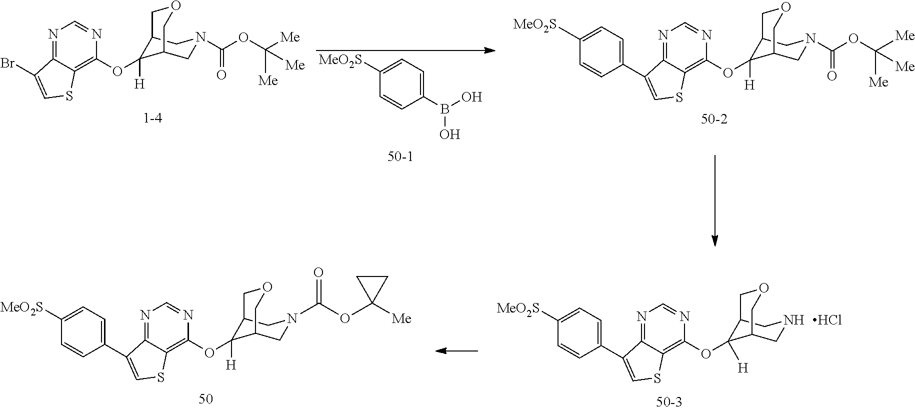 [SMILES: CC(C)(C)OC(=O)N1CC2COCC(C1)C2Oc1ncnc2c(Br)csc12 ; CS(=O)(=O)c1ccc(B(O)O)cc1 ; CC(C)(C)OC(=O)N1CC2COCC(C1)C2Oc1ncnc2c(-c3ccc(S(C)(=O)=O)cc3)csc12 ; CC1(OC(=O)N2CC3COCC(C2)C3Oc2ncnc3c(-c4ccc(S(C)(=O)=O)cc4)csc23)CC1 ; CS(=O)(=O)c1ccc(-c2csc3c(OC4C5CNCC4COC5)ncnc23)cc1 ; Cl]

Step 1: In similar fashion to Example 1, Step 3, Compound 1-4 was converted to Compound 50-2.

Steps 2 and 3. In similar fashion to Example 3, Compound 50-2 was converted to Compound 50, a white solid, LC-MS: m/e 530 (M+1).

### Example 51

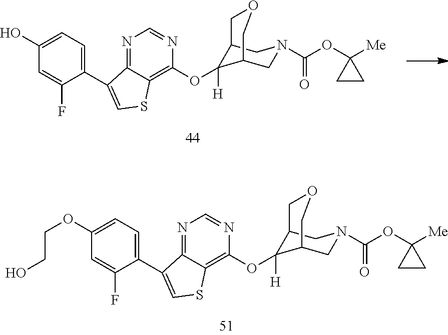 [SMILES: CC1(OC(=O)N2CC3COCC(C2)C3Oc2ncnc3c(-c4ccc(O)cc4F)csc23)CC1 ; CC1(OC(=O)N2CC3COCC(C2)C3Oc2ncnc3c(-c4ccc(OCCO)cc4F)csc23)CC1]

Compound 44 (0.090 g, 0.19 mmol) was combined with 2-bromoethanol (0.092 g, 0.74 mmol) and K₂CO₃ (0.102 g, 0.74 mmol) in DMF (2.0 mL) and heated at 120° C. 4 h. Concentration and purification by PLC yielded Compound 51, a white solid, LC-MS: m/e 530 (M+1).

### Example 52

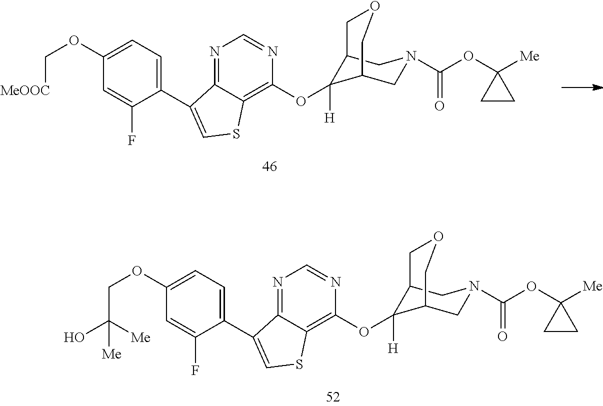 [SMILES: COC(=O)COc1ccc(-c2csc3c(OC4C5COCC4CN(C(=O)OC4(C)CC4)C5)ncnc23)c(F)c1 ; CC(C)(O)COc1ccc(-c2csc3c(OC4C5COCC4CN(C(=O)OC4(C)CC4)C5)ncnc23)c(F)c1]

Compound 46, (crude, prepared from 0.100 g, 0.21 mmol Compound 44) in THF (2.0 mL) at 0° C. was treated with MeMgBr (3.0M in ether, 0.27 mL, 0.81 mmol), stirred 3 h, quenched with water and extracted with EtOAc. Drying (MgSO₄), concentration and purification by PLC yielded Compound 52 as a yellow solid, LC-MS: m/e 558 (M+1).

### Example 53

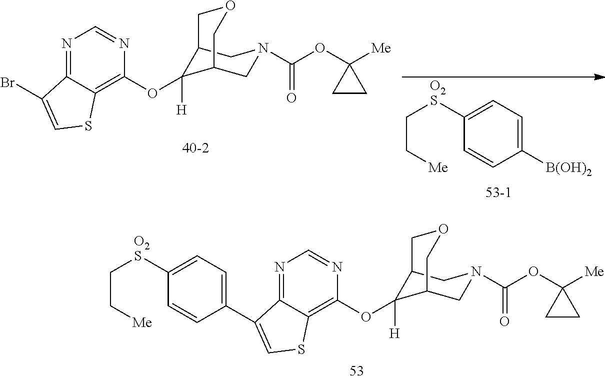 [SMILES: CC1(OC(=O)N2CC3COCC(C2)C3Oc2ncnc3c(Br)csc23)CC1 ; CCCS(=O)(=O)c1ccc(B(O)O)cc1 ; CCCS(=O)(=O)c1ccc(-c2csc3c(OC4C5COCC4CN(C(=O)OC4(C)CC4)C5)ncnc23)cc1]

In similar fashion to Example 1, Step 3, Compound 40-2 was converted to Compound 53, a white solid, LC-MS: m/e 558 (M+1).

### Example 54

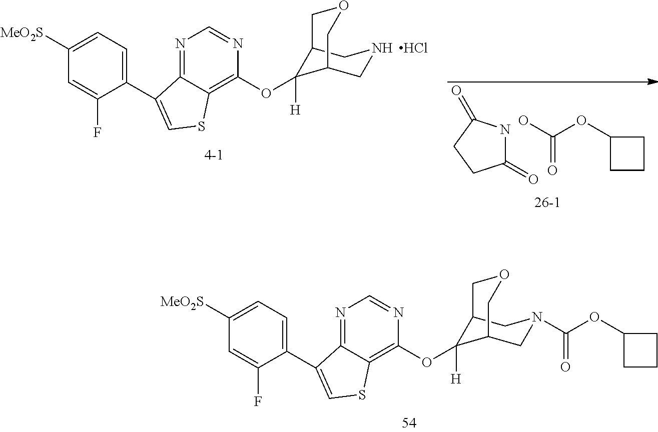

In similar fashion to Example 3, Step 2, Compound 4-1 was treated with Compound 26-1 to provide Compound 54, a white solid, LC-MS: m/e 548 (M+1).

### Example 55

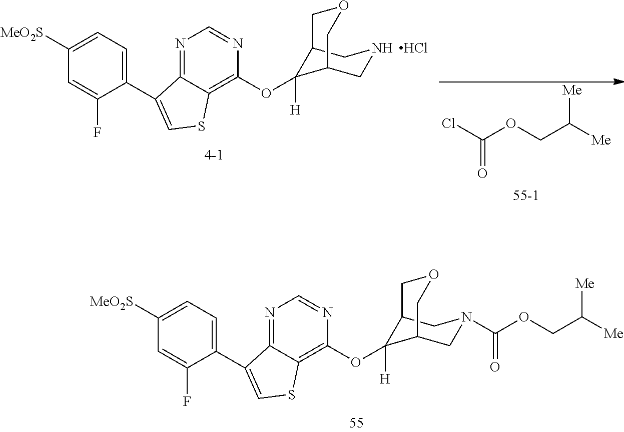 [SMILES: CS(=O)(=O)c1ccc(-c2csc3c(OC4C5CNCC4COC5)ncnc23)c(F)c1 ; Cl ; CC(C)COC(=O)Cl ; CC(C)COC(=O)N1CC2COCC(C1)C2Oc1ncnc2c(-c3ccc(S(C)(=O)=O)cc3F)csc12]

In similar fashion to Example 3, Step 2, Compound 4-1 was treated with Compound 55-1 to provide Compound 55, a white solid, LC-MS: m/e 550 (M+1).

### Example 56

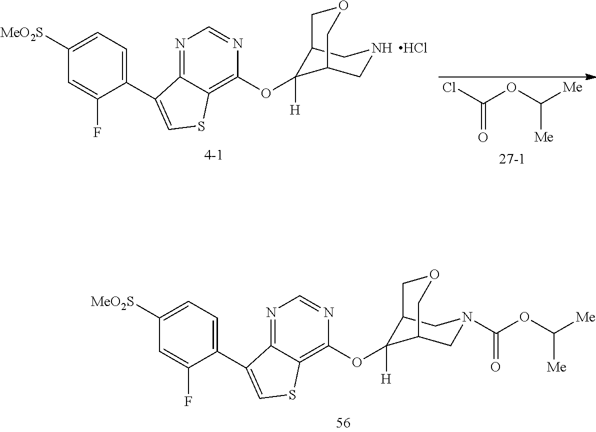 [SMILES: CS(=O)(=O)c1ccc(-c2csc3c(OC4C5CNCC4COC5)ncnc23)c(F)c1 ; Cl ; CC(C)OC(=O)Cl ; CC(C)OC(=O)N1CC2COCC(C1)C2Oc1ncnc2c(-c3ccc(S(C)(=O)=O)cc3F)csc12]

In similar fashion to Example 3, Step 2, Compound 4-1 was treated with Compound 27-1 to provide Compound 56, a white solid, LC-MS: m/e 536 (M+1).

### Example 57

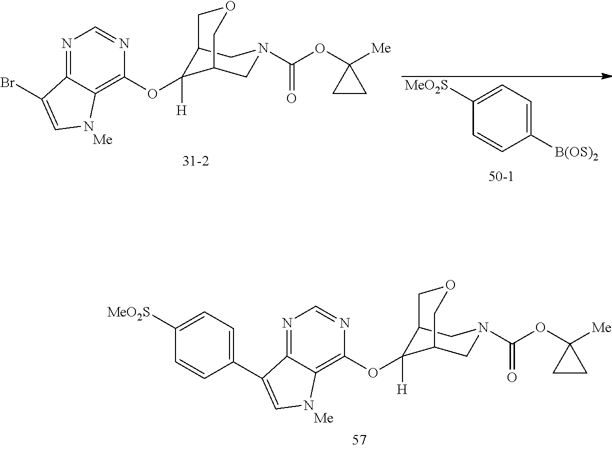

Step 3: In similar fashion to Example 1, Step 3, Compound 31-2 was converted to Compound 57, a yellow solid, LC-MS: m/e 527 (M+1).

### Biological Assay

The activity of these compounds may be assayed in cells transfected the GPR119 receptor (human, mouse, rat or monkey). Incubation with the above compounds results in an increase in intracellular cAMP, from which an EC₅₀ value may be calculated. Ki values for compounds at this receptor can be determined by employing a radio-labeled agonist. In vivo activity can be determined by conducting an oral glucose-tolerance test in an appropriate species, such as mouse. The table below shows human cAMP, from which EC₅₀ values have been calculated.

| | h-cAMP |
|---|---|
| Example | EC₅₀, μM |
| 1 | 0.008 |
| 2 | 0.062 |
| 3 | 0.009 |
| 4 | 0.023 |
| 5 | 0.018 |
| 6 | 0.016 |
| 7 | 0.047 |
| 8 | 0.150 |
| 9 | 0.047 |
| 10 | 0.024 |
| 11 | 0.018 |
| 12 | 0.086 |
| 14 | 0.298 |
| 16 | 0.247 |
| 17 | 0.013 |
| 19 | 0.0032 |
| 21 | 0.0053 |
| 22 | 0.0020 |
| 23 | 0.0011 |
| 24 | 0.0012 |
| 25 | 0.00046 |
| 27 | 0.00076 |
| 28 | 0.0021 |
| 29 | 0.00048 |
| 30 | 0.00074 |
| 31 | 0.0084 |
| 32 | 0.0057 |
| 33 | 0.0010 |
| 34 | 0.072 |
| 35 | 0.045 |
| 36 | 0.035 |
| 37 | 0.0016 |
| 38 | 0.00044 |
| 39 | 0.00036 |
| 40 | 0.00051 |
| 41 | 0.00047 |
| 42 | 0.0010 |
| 43 | 0.00038 |
| 44 | 0.0010 |
| 45 | 0.0001 |
| 46 | 0.0025 |
| 47 | 0.0010 |
| 48 | 0.0014 |
| 49 | 0.0017 |
| 50 | 0.0022 |
| 51 | 0.00065 |
| 52 | 0.00050 |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in responsiveness of the mammal being treated for any of the indications with the compounds of the invention indicated above. The specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1-42. (canceled)

43. A compound having the formula I: 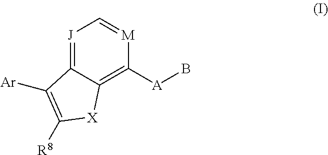
or a pharmaceutically acceptable salt thereof, wherein:
A is —O—, —S—, —NH—, or —N(C₁-C₆alkyl)-;
B is: 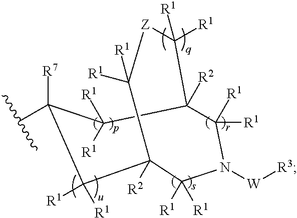
J is —C(R¹¹)— or —N—;
M is —C(R¹¹)— or —N—;
W is a bond, C₁-C₆alkyl, —C(O)—, —C(O)—O—, —S(O)₂—, —S(O)₂—N(R¹⁰)—, C(S)O or —C(O)—N(R¹⁰)—;
X is —O—, —S—, —NH, —N(C₁-C₆alkyl), —N(cycloalkyl), —N(hydroxyalkyl) or —N(hydroxyaryl);
Z is a bond, —C(O)—, —C═NOR¹², —C═C(R¹⁴)₂, —C(R¹)₂—, —O—, —N(R¹⁰)— or —S(O)ₙ—;
each occurrence of R¹ is independently hydrogen, C₁-C₆alkyl, cycloalkyl, halogen, haloalkyl or —OR⁷, wherein OR⁷ is not adjacent to —N—W—R³;
each occurrence of R² is independently hydrogen or C₁-C₆alkyl;
R³ is C₁-C₆alkyl, C₂-C₆alkenyl, C₂-C₆alkynyl, haloalkyl, —(C₁-C₆alkyl)ₜ-cycloalkyl, —(C₁-C₆alkyl)ₜ-heterocycloalkyl, —(C₁-C₆alkyl)ₜ-aryl or —(C₁-C₆alkyl)ₜ-heteroaryl, wherein the cycloalkyl, heterocycloalkyl, aryl or heteroaryl group can be unsubstituted or substituted with one or more substituents each independently selected from R⁹;
each occurrence of R⁴ is independently hydrogen or C₁-C₆alkyl;
each occurrence of R⁷ is independently hydrogen or C₁-C₆alkyl;
Ar is aryl, heteroaryl, heterocycloalkyl or cycloalkyl, any of which can be unsubstituted or substituted with one or more substituents each independently selected from R⁹;
R⁸ is hydrogen, halogen, C₁-C₆alkyl or cycloalkyl;
R⁹ represents C₁-C₆alkyl, C₂-C₆alkenyl, C₂-C₆alkynyl, halogen, haloalkyl, —CN, —NO₂, —O—(C₁-C₆alkyl)ₜ-R¹³, —S—(C₁-C₆alkyl)ₜ-R¹³, —N(R¹³)—(C₁-C₆alkyl)ₜ-R¹³, —(C₁-C₆alkyl)ₜ-R¹³, —C(O)—(C₁-C₆alkyl)ₜ-R¹³, —C(O)O—(C₁-C₆alkyl)ₜ-R¹³, —N(R⁷)C(O)—(C₁-C₆alkyl)ₜ-R¹³, —C(O)N(R⁷)—(C₁-C₆alkyl)ₜ-R¹³, —OC(O)—(C₁-C₆alkyl)ₜ-R¹³, —N(R⁷)C(O)N(R⁷)—(C₁-Colkyl)ₜ-R¹³, —N(R⁷)C(O)O—(C₁-C₆alkyl)ₜ-R¹³, —S(O)—(C₁-C₆alkyl)ₜ-R¹³ or —S(O)₂(C₁-C₆alkyl)ₜ-R¹³;
R¹⁰ is hydrogen, C₁-C₆alkyl, aryl, or —C(O)OR⁴;
each occurrence of R¹¹ is independently hydrogen, C₁-C₆alkyl, aryl, cycloalkyl, heterocycloalkyl, heteroaryl, —N(R⁷)₂ or halogen;
each occurrence of R¹² is independently hydrogen, C₁-C₆alkyl or aryl;
each occurrence of R¹³ is independently hydrogen, hydroxyl, haloalkyl, aryl, cycloalkyl, —COOC₁-C₆alkyl, —OC₁-C₆alkyl or heteroaryl;
each occurrence of R¹⁴ is independently hydrogen, C₁-C₆alkyl or aryl, or both R¹⁴ groups, and the carbon atom to which they are attached, combine to form a cycloalkyl or heterocycloalkyl group;
each occurrence of n, p, q, r, s, t and u is independently 0, 1 or 2.

44. The compound of claim 43, wherein A is —O—.

45. The compound of claim 43, wherein B is:

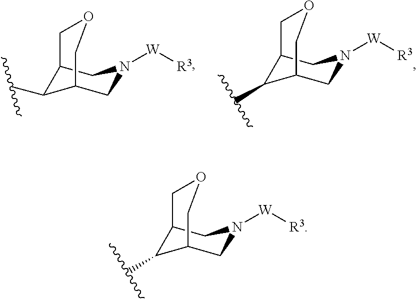

46. The compound of claim 43, wherein B is: 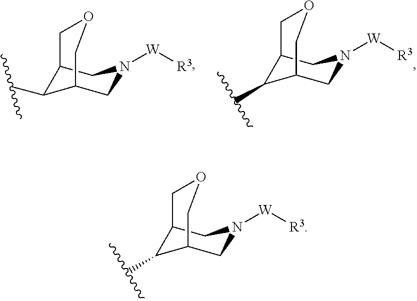
wherein R³ is C₁-C₆alkyl, haloalkyl, cycloalkyl or heteroaryl, wherein the cycloalkyl and heteroaryl are substituted with one or more substituents selected from R⁹.

47. The compound of claim 43, wherein W is —C(O)O— or —C(O)—.

48. The compound of claim 43, wherein W is a bond.

49. The compound of claim 43, wherein R³ is C₁-C₆alkyl, haloalkyl, heteroaryl or cycloalkyl.

50. The compound of claim 43, wherein Ar is aryl or heteroaryl.

51. The compound of claim 43, wherein Ar is phenyl.

52. The compound of claim 43, wherein Ar is pyridyl.

53. The compound of claim 43, wherein Ar is:

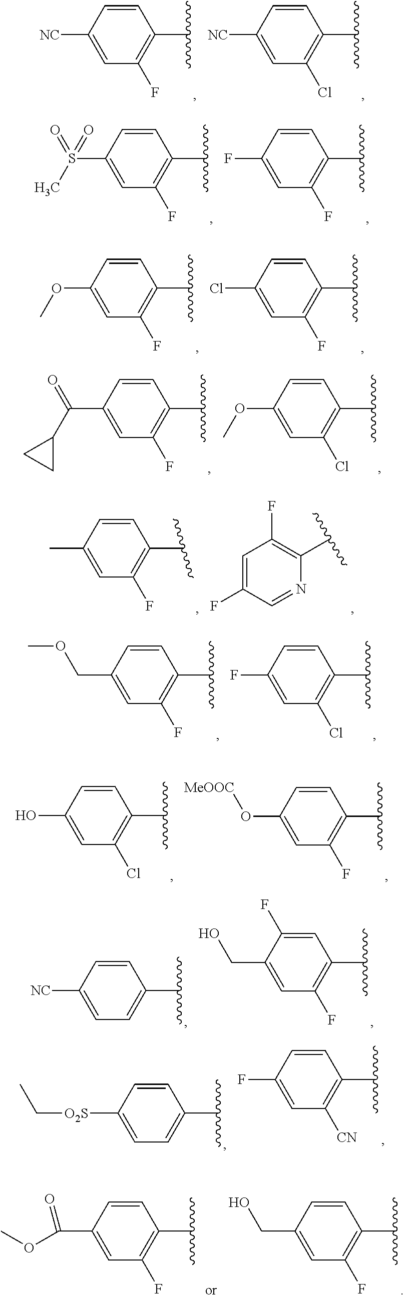

54. The compound of claim 43, wherein J and M are each —N—.

55. The compound of claim 43, wherein A is —O— and W is —C(O)O—, a bond or —CH₂—.

56. The compound of claim 43, which is of formula Ia 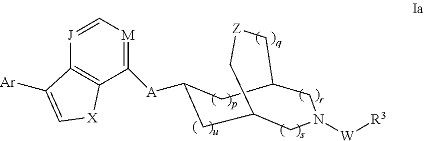
or pharmaceutically acceptable salt thereof, wherein:
J and M are independently —N— or —C(R¹¹)—, wherein R₁₁ is hydrogen, C₁-C₆alkyl, aryl, cycloalkyl, heterocycloalkyl, heteroaryl, —N(R⁷)₂ or halogen, wherein one of J or M must be —N—;
W is a bond, C₁-C₆alkyl, —C(O)—, —C(O)O—, —C(S)O—, or —SO₂;
X is —O—, —S—, —NH, —N(C₁-C₆alkyl), —N(cycloalkyl) or —Nhydroxyaryl;
A is —O—, —S—, —NH or —N(C₁-C₆alkyl);
Z is a bond, —O—, —S(O)ₙ, —NH, N(C₁-C₆alkyl), N-cycloalkyl or —C(O)—;
Ar is phenyl or heteroaryl, wherein the phenyl or heteroaryl is unsubstituted or substituted with one or more substituents each independently selected from the group consisting of C₁-C₆alkyl, cycloalkyl, haloalkyl, halogen, —O—C₁-C₆alkyl, —OH, —CN, COC₁-C₆alkyl, —C(O)O—C₁-C₆alkyl, —CO-cycloalkyl, S(O)₂C₁-C₆alkyl, S(O)₂cycloalkyl, and heteroaryl;
R³ is C₁-C₆alkyl, cycloalkyl, haloalkyl or heteroaryl, wherein cycloalkyl, haloalkyl or heteroaryl are unsubstituted or substituted with one or more substituents each independently selected from the group consisting of C₁-C₆alkyl, halogen, OC₁-C₆alkyl, cycloalkyl, haloalkyl and —CN;
R⁷ is hydrogen, C₁-C₆alkyl or halogen;
n=0, 1 or 2;
p=0, 1 or 2;
q=0, 1 or 2;
r=0, 1 or 2, wherein when r=0, then s is non-zero
s=0, 1 or 2;
u=0, 1 or 2.

57. A compound of formula Ib 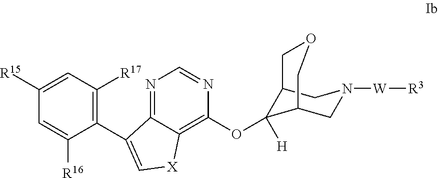
or pharmaceutically acceptable salt thereof, wherein:
W is a bond, C₁-C₆alkyl, —C(O)—, —C(O)O—, —C(S)O— or —SO₂;
X is —O—, —S—, —NH or —N(C₁-C₆alkyl);
R³ is C₁-C₆alkyl, cycloalkyl, haloalkyl or heteroaryl, wherein cycloalkyl is unsubstituted or substituted with 1 to 3 substituents each independently selected from the group consisting of C₁-C₆alkyl, halogen, OC₁-C₆alkyl, and CO(O)C₁-C₆alkyl, wherein the heteroaryl is unsubstituted or substituted with 1 to 3 substituents each independently selected from the group consisting of C₁-C₆alkyl, cycloalkyl, haloalkyl, halogen, —CN and OC₁-C₆alkyl, and wherein the haloalkyl is unsubstituted or substituted with 1 to 3 substituents each independently selected from the group consisting of haloalkyl;
R¹⁵ is C₁-C₆alkyl, cycloalkyl, haloalkyl, halogen, —O—C₁-C₆alkyl, —CN, —COC₁-C₆alkyl, —C₁-C₆alkyl-OH, —C(O)O—C₁-C₆alkyl, —CO-cycloalkyl, —S(O)₂C₁-C₆alkyl, —S(O)₂cycloalkyl, or heteroaryl
R¹⁶ is halogen; and
R¹⁷ is hydrogen or halogen.

58. A compound of claim 57 or pharmaceutically acceptable salt thereof, wherein X is —S—.

59. A compound of claim 57 or pharmaceutically acceptable salt thereof, wherein X is —NC₁-C₆alkyl-.

60. A compound or a pharmaceutically acceptable salt thereof, selected from the group consisting of:

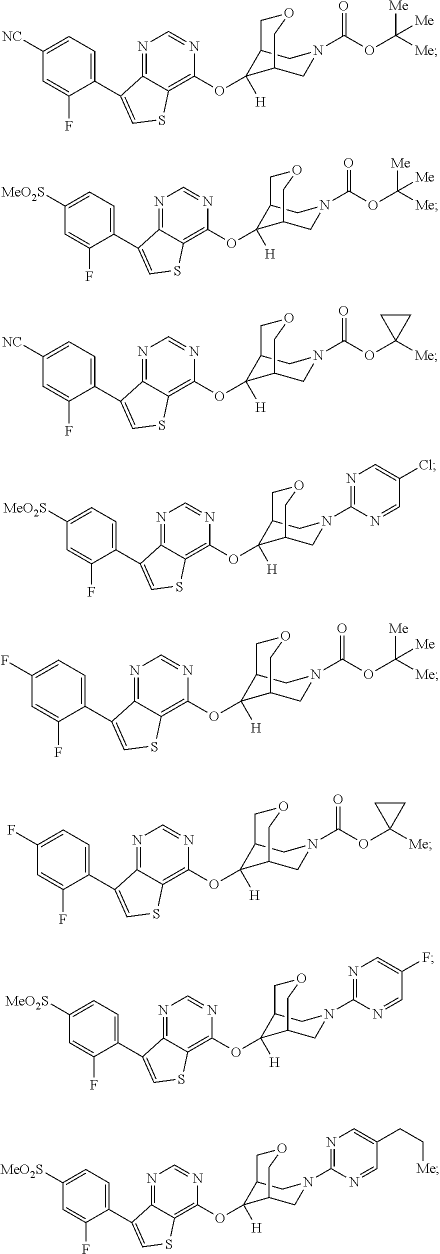

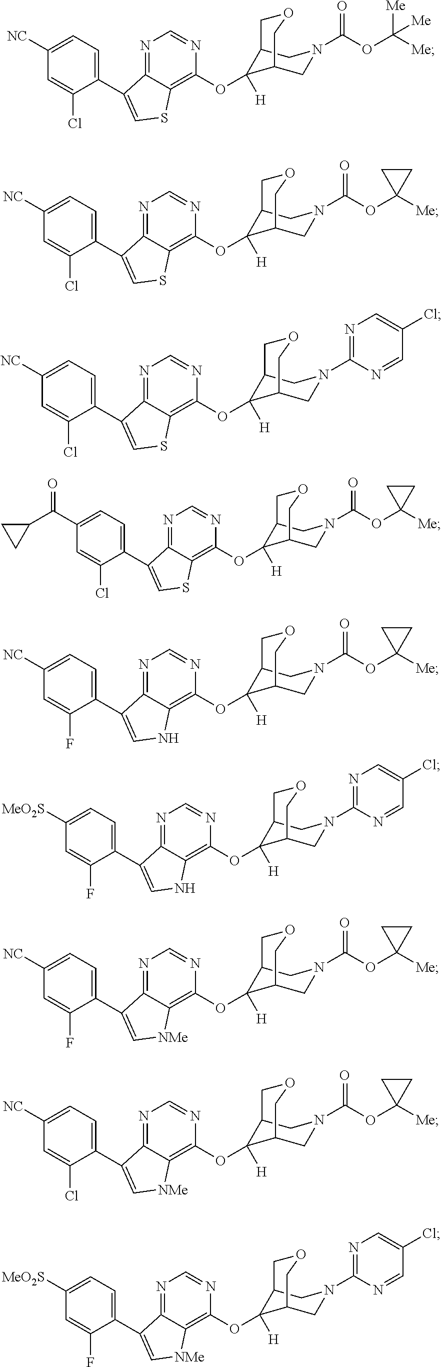

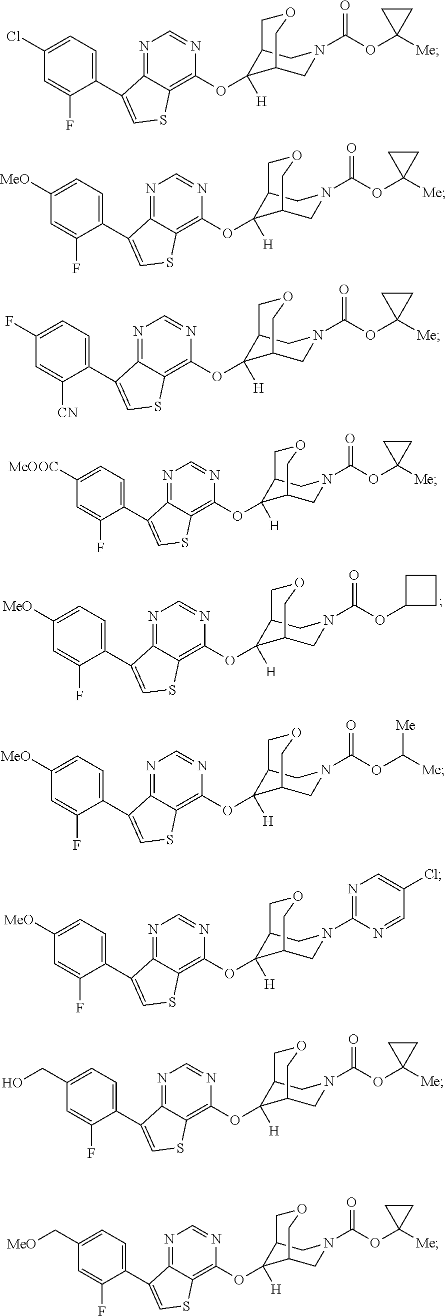

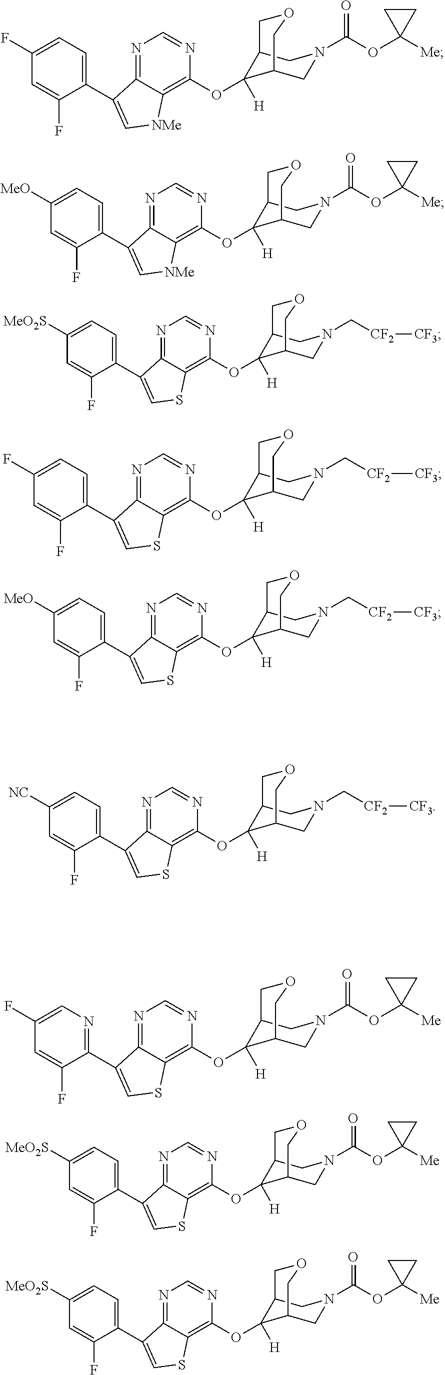

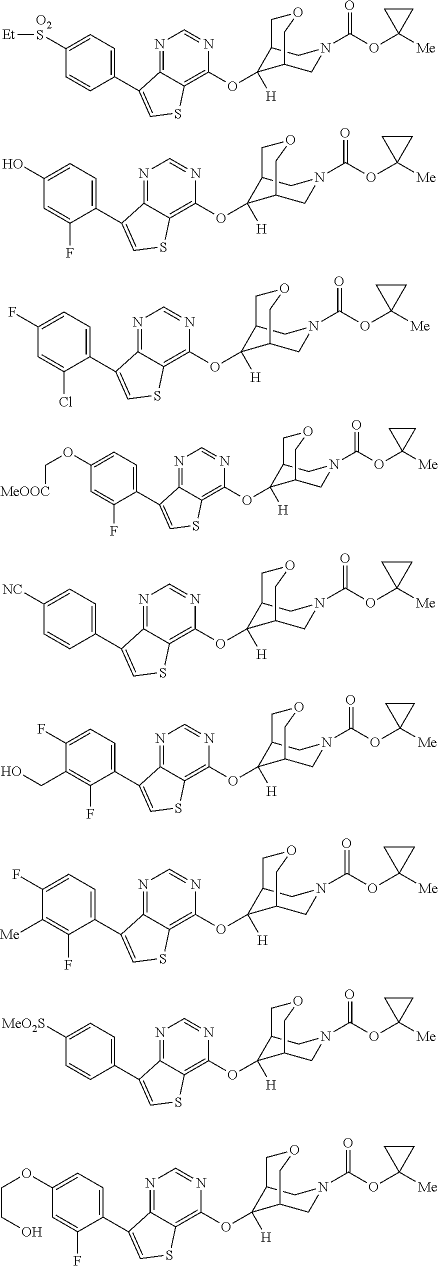

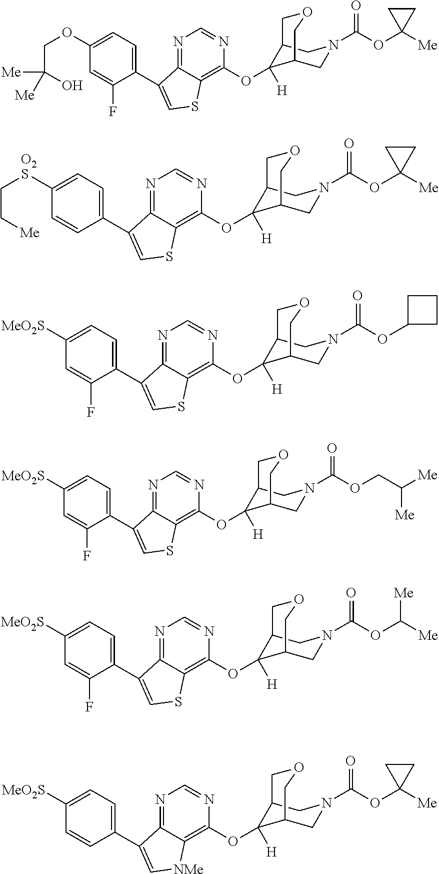 [SMILES: CC(C)(O)COc1ccc(-c2csc3c(OC4C5COCC4CN(C(=O)OC4(C)CC4)C5)ncnc23)c(F)c1 ; CCCS(=O)(=O)c1ccc(-c2csc3c(OC4C5COCC4CN(C(=O)OC4(C)CC4)C5)ncnc23)cc1 ; CS(=O)(=O)c1ccc(-c2csc3c(OC4C5COCC4CN(C(=O)OC4CCC4)C5)ncnc23)c(F)c1 ; CC(C)COC(=O)N1CC2COCC(C1)C2Oc1ncnc2c(-c3ccc(S(C)(=O)=O)cc3F)csc12 ; CC(C)OC(=O)N1CC2COCC(C1)C2Oc1ncnc2c(-c3ccc(S(C)(=O)=O)cc3F)csc12 ; Cn1cc(-c2ccc(S(C)(=O)=O)cc2)c2ncnc(OC3C4COCC3CN(C(=O)OC3(C)CC3)C4)c21]

61. A pharmaceutical composition comprising a compound of claim 43, or a pharmaceutically acceptable salt thereof, and a pharmaceutically acceptable carrier.

62. A method for the treatment of a condition selected from the group consisting of obesity and diabetes comprising administering to an individual a pharmaceutical composition comprising the compound of claim 43.
